(19) United States
(12) Patent Application Publication
(10) Pub. No.: US 2026/0265682 A1
(43) Pub. Date: Sep. 10, 2026

(54) Microorganisms providing growth promotion phenotypes to plants

(71) Applicant: Agriculture Victoria Services PTY LTD, Bundoora, VICTORIA (AU)

(72) Inventors: Rens Van Essen, Fitzroy North (AU); Holly Hone, Albert Park (AU); Ankush Chandel, Kingsbury (AU); Tongda Li, Alphington (AU); Timothy Ivor Sawbridge, Coburg (AU); Ross C. Mann, Coburg (AU); Jatinder Kaur, Taylors Hill (AU); German Carlos Spangenberg, Bundoora (AU)

(73) Assignee: Agriculture Victoria Services PTY LTD, Bundoora, VICTORIA (AU)

(21) Appl. No.: 18/875,609

(22) PCT Filed: Jun. 26, 2023
(86) PCT No.: PCT/AU2023/050582

§ 371 Date: Dec. 16, 2024

(30) Foreign Application Priority Data
Jun. 24, 2022 (AU) 2022901766

(51) Int. Cl. C12N 1/205; A01H 3/00; A01H 17/00; A01N 63/20; A01P 21/00; C07K 14/195; C12Q 1/04; C12Q 1/68; C12R 1/01

(52) U.S. Cl. CPC C12N 1/205; A01H 3/00; A01H 17/00; A01N 63/20; A01P 21/00; C07K 14/195; C12Q 1/04; C12Q 1/68; C12R 2001/01

(57) ABSTRACT

The present invention relates to plant growth promoting endophytes, preferably of plants of the Glycine genus, particularly plant growth promoting Curtobacterium sp. endophytes. The present invention also relates to seeds, plants, and parts thereof infected with such endophytes; and related methods, including methods for conferring plant growth promotion to plants and for selecting plant growth promoting endophytes.

### FIELD OF THE INVENTION

The present invention relates to novel plant growth promoting endophytes, preferably of plants of the Glycine genus. The present invention also relates to seeds, plants, and parts thereof infected with such endophytes; and related methods, including methods for conferring growth promotion to plants and for selecting plant growth promoting endophytes.

### BACKGROUND OF THE INVENTION

The agricultural industry faces unparalleled supply and demand challenges given that the global demand for food and food products continues to increase while the availability of agricultural resources (e.g., arable land, water, access to technology) steadily declines. Population and consumer expectations are on the rise. Indeed, the world population has quadrupled over the last century, and sustained growth is forecasted for the foreseeable future. Moreover, rising salaries, particularly in developing nations, is accompanied by changes in consumer diets (e.g., increased consumption of protein and meat). The cumulative effects of these evolutions places greater demands on the agricultural industry in terms of both the quantity and quality of farmed products.

To compound the strains on farm crops, population growth also drives urbanisation, effectively decreasing the amount of arable land. In addition, crop yields are now threatened by climate change, which may shorten the growing season and expose vulnerable crops to prolonged periods of heat, drought, or high winds, thereby reducing yields.

While clearing forests may afford larger quantities of arable land, such practices are known to have detrimental effects on the environment and would only exacerbate climate change-induced threats to crop yields. Consequently, techniques designed to improve the quality of existing arable land and/or crop cultivars have been designed and implemented in an effort to mitigate an otherwise impending global food crisis. Specifically, plant-associated microbes (i.e., bacteria, fungi, viruses, and archaea) that possess an innate ability to confer beneficial properties to plant species have been identified and utilised to improve crop yields in a variety of common crop cultivars (e.g., maize, rice, soybean, wheat, and the like).

Plant-associated microbes may, for example, improve a host plant's tolerance to any number of biotic (e.g., pathogenic microorganisms) or abiotic (e.g., water, temperature, salinity, or nutrient) stresses and allow the plant to produce a higher crop yield and/or to remain productive over a longer period. Soil type and plant genotype are known to stimulate the assembly of plant-associated microbial communities. However, intense plant domestication practices have eradicated the symbiotic associations between beneficial microbes and many common crop cultivars (e.g., maize, wheat, rice, and common bean). Similar shifts have been observed in the seed microbiomes of domesticated crop cultivars, and since little is known about the seed microbiomes of wild crop cultivars, the utility of seed-associated microbes as an adjunct agricultural resource is compromised.

Seeds generally comprise a diverse microbiome of epiphytic and endophytic microorganisms that are readily amenable to vertical transmission across generations of plants. Furthermore, seed-associated microbes can act as primary plant inoculants and affect seed germination and initial plant vigour, as well as provide biotic-abiotic stress tolerance. Once adapted to the physiological changes, the seed microbes inhabiting seed tissues can colonise seedlings and surrounding rhizospheres during seed germination. Unfortunately, however, seed-associated bacteria have proven difficult to cultivate and thus are not routinely used to improve crop yields in any common crop cultivars.

Consequently, there exists a need to overcome, or at least alleviate, one or more of the difficulties or deficiencies associated with the prior art, and in particular there remains a need for improved growth in plants and methods that promote such growth.

### SUMMARY OF THE INVENTION

In one aspect, the present invention provides a substantially purified or isolated Curtobacterium sp. endophyte, wherein the endophyte is capable of conferring a plant growth promotion phenotype to the seed, plant, or part thereof from which it is substantially purified or isolated and/or is capable of conferring a plant growth promotion phenotype to a plant or part thereof to which it is inoculated.

Preferably, the endophyte is substantially purified or isolated from a plant of the Glycine genus.

In preferred embodiments, substantially purified or isolated Curtobacterium sp. endophyte is a strain denoted P3-Gcland-NS-Dand-IS-96-1 (Curtobacterium P3GeND-IS-96-1), P3-Gcland-NS-Dand-IS-26-1 (Curtobacterium P3GeND-IS-26-1), P2-Gtab-NS-Kalkallo-IS-65-1 (Curtobacterium P2GtNK-IS-65-1), P2-Gtab-NS-Kalkallo-IS-64-1 (Curtobacterium P2GtNK-IS-64-1), P3-Gtab-NS-Kalkallo-IS-74-1 (Curtobacterium P3GtNK-IS-74-1) and/or P3-Gcland-NS-Dand-IS-27-1 (Curtobacterium P3GeND-IS-27-1) as deposited with the National Measurement Institute of 1/153 Bertie St, Port Melbourne, Victoria 3207 Australia on 14 Jun. 2022 with accession number V22/011283, V22/011278, V22/011281, V22/011280, V22/011282 and V22/011279, respectively.

The present invention also provides variants of the Curtobacterium sp. endophyte strains as hereinbefore described. Such variants include naturally occurring allelic variants and non-naturally occurring variants. Non-naturally occurring variants may have artificially introduced genetic variation. The genetic variation may be introduced utilising any standard techniques, e.g., via one or more of random mutagenesis, di/poly-ploidisation, targeted mutagenesis; cisgenesis; transgenesis; intragenesis.

Additions, deletions, substitutions and derivatisations of one or more of the nucleotides in the genome of the endophyte strains are contemplated, so long as the modifications do not result in loss of functional activity of the variant. In some cases such modifications may increase functional activity of the variant.

Preferably the variant has at least approximately 95% sequence identity to the genome of the endophyte strain of the invention, more preferably at least approximately 97% identity, even more preferably at least approximately 98% identity, most preferably at least approximately 99% identity. Such functionally active variants include, for example, those having conservative nucleic acid changes in the genome.

By “conservative nucleic acid changes” as used herein, is meant nucleic acid substitutions that result in conservation of the amino acid in the encoded protein owing to the degeneracy of the genetic code. Such functionally active variants also include, for example, those having nucleic acid changes which result in conservative amino acid substitutions of one or more residues in the corresponding amino acid sequence.

By “conservative amino acid substitutions”, is meant the substitution of an amino acid by another one of the same class, the classes being as follows:
Nonpolar: Ala, Val, Leu, Ile, Pro, Met Phe, Trp
Uncharged polar: Gly, Ser, Thr, Cys, Tyr, Asn, Gln
Acidic: Asp, Glu
Basic: Lys, Arg, His

Other conservative amino acid substitutions may also be made as follows:
Aromatic: Phe, Tyr, His
Proton Donor: Asn, Gln, Lys, Arg, His, Trp
Proton Acceptor: Glu, Asp, Thr, Ser, Tyr, Asn, Gln

By “endophyte” is meant an organism, generally a microorganism (i.e., bacteria, fungi, viruses, and archaea) that co-exists in a mutually beneficial relationship with a plant. Endophytes generally live on, in, or otherwise in close proximity to a plant and rely on the plant for survival, while simultaneously conferring a certain benefit to the plant. For example, an endophyte may confer enhanced biotic (e.g., pathogenic microorganisms) or abiotic (e.g., water, temperature, salinity, or nutrient) stress tolerance to a plant host. By ‘plant host’ as used herein, we mean the plant with which the endophyte is associated. Endophytes of Curtobacterium sp. are bacterial endophytes.

In some embodiments, the endophyte lives on a plant to which it provides benefit. In other embodiments, the endophyte lives in a plant to which it provides benefit. In yet other embodiments, the endophyte lives in close proximity to a plant to which it provides benefit.

By “substantially purified” as used in the context of an endophyte, we mean that the endophyte is free of other organisms. The term includes, for example, an endophyte in axenic culture. Preferably, the endophyte is at least approximately 90% pure, more preferably at least approximately 95% pure, even more preferably at least approximately 98% pure, even more preferably at least approximately 99% pure.

By “isolated” as used in the context of an endophyte, we mean that the endophyte is removed from its original environment (e.g., the natural environment if it is naturally occurring; e.g., the plant). For example, a naturally occurring endophyte present in nature in a living plant is not isolated, but the same endophyte separated from some or all of the coexisting materials in the natural environment is isolated.

A plant of the Glycine genus includes plant seeds and plant parts thereof and may also be known as a soybean, soja bean, or soya bean plant.

In a preferred embodiment, the plant of the Glycine genus from which a Curtobacterium sp. endophyte is substantially purified or isolated is a Glycine max, Glycine clandestina, Glycine tomentella, Glycine tabacina, Glycine canescens, Glycine latrobeana, Glycine microphylla, Glycine albicans, Glycine aphyonota, or Glycine soja species plant, more preferably G. max.

The Curtobacterium sp. endophyte may be substantially purified or isolated from any particular part of the plant, e.g., an organ. In preferred embodiments, the endophyte is substantially purified or isolated from a flower, flower bract, leaf, petiole, stem, seed, seedpod, or root of the plant, more preferably a seed or seedpod.

The present invention arises from the discovery of Curtobacterium sp. endophyte strains from plants of the Glycine genus and their ability to form mutually beneficial relationships with plants that may be used to confer certain benefits to the plants. In particular, the present invention arises further from the surprising discovery that the endophytes may promote plant growth and that certain species or strains of the Curtobacterium genus are particularly plant growth promoting and may be inoculated into a plant that is otherwise absent the endophytes to confer a plant growth promotion phenotype to a seed, plant, or part thereof, including for example continued growth under abiotic stress conditions.

By “inoculated” is meant to be placed in association with a plant to form a mutually-beneficial relationship with the plant, whether that be on, in, or otherwise in close proximity to the plant. In preferred embodiments, the plant or part thereof to which the endophyte is inoculated is first free of that endophyte.

In general, a plant growth promoting endophyte possesses genetic and/or metabolic characteristics that result in a plant growth promotion (PGP) phenotype in a plant harbouring, or otherwise associated with, the endophyte. The PGP phenotype may include improved enhanced abiotic stress tolerance and/or enhanced vigour in the plant with which the endophyte is associated, relative to a plant not associated with the endophyte, or instead associated with a control endophyte such as a Curtobacterium flaccumfaciens bacterial strain. The abiotic stress may include, but is not limited to, nutrient (e.g., nitrogen, phosphorous, potassium, magnesium, sulphur, and/or calcium), water (e.g., mild or severe drought or rain), and/or temperature stress (e.g., excessively high or low temperatures, including frosts).

In a preferred embodiment, the endophyte is capable of conferring a PGP phenotype to the seed, plant, or part thereof from which it is substantially purified or isolated, and/or is capable of conferring a PGP phenotype to a seed, plant, or part thereof to which it is inoculated. For clarity, an endophyte which presents with in vitro PGP activity, for example a water sequestration activity, may be taken to be capable of conferring the associated phenotype to a plant, for example resistance to dehydration when the plant is exposed to drought.

In preferred embodiments, the PGP phenotype is characterised by enhanced growth of the plant containing and/or inoculated with the endophyte. For example, a PGP phenotype may increase crop yield or plant vigour in a plant harbouring the endophyte by improving plant uptake and/or utilisation water and/or nutrients (nitrogen, phosphorous, potassium, magnesium, sulphur, and/or calcium). In a particularly preferred embodiment, the PGP phenotype may improve plant uptake and/or utilisation of water and/or phosphorous.

Alternatively, the PGP phenotype may be characterised by enhanced growth of a plant containing and/or inoculated with the endophyte under one or more abiotic stress condition(s). In preferred embodiments, the PGP phenotype is characterised by improved tolerance to a water and/or nutrient stress (e.g., limited availability of nitrogen, phosphorous, potassium, magnesium, sulphur, and/or calcium), particularly limited availability of water and/or phosphorous.

The improved plant growth promotion which in these embodiments the endophyte is capable of conferring may generally be considered as compared to the plant growth promotion, or lack thereof as the case may be, of a plant or part thereof that is absent of the endophyte (“no endophyte control”), and/or as compared to the plant growth promotion of a plant that contains a Curtobacterium flaccumfaciens species bacterial strain, which again may be taken from a presented in vitro plant growth promotion activity or an observed in planta plant growth promotion activity.

In preferred embodiments, the PGP phenotype may be characterised by a gene encoding one or more proteins (e.g., enzymes or the like) which facilitate the uptake and/or utilisation water and/or nutrients. In particularly preferred embodiments, the PGP phenotype may be characterised by a gene encoding phosphate solubilisation (e.g., ugd, gcd, or gad), phosphonate solubilisation (e.g., phnA, phnB, phnC, phnD, phnE, phnW, phnX, ppd, or pepM), phosphate transport (e.g., pstA, pstB, pstC, pstS, phoP, or phoR), or any combination thereof.

In preferred embodiments, the plant growth promotion phenotype may include at least one gene selected from the group consisting of the sequences shown in FIGS. 10 to 71, hereto (SEQ ID NOs: 8-18, 20-23, 25-28, 30-33, 35-40, 42-47, 49-54, 56-61, 63-65, and 66-68), or a sequence having at least approximately 95%, preferably 97%, more preferably 98%, more preferably 99% sequence identity to the full length of at least one of SEQ ID NOs: 8-18, 20-23, 25-28, 30-33, 35-40, 42-47, 49-54, 56-61, 63-65, and 66-68.

The seed, plant, or part thereof to which the endophyte is capable of conferring a PGP phenotype may be any plant which contributes to the global food supply as, for example, crop cultivars harvested for human consumption or for use in medicinal and/or food products, or as forage for grazing livestock. As such, this includes plants of many important food and horticultural crops, such as plants of the Fabaceae, Poaceae, Apiaceae, Cruciferae, Solanaceae, Cucurbitaceae, Amaryllidaceae, Malvaceae, Lamiaceae, Rosaceae, Rutaceae or Brassicaceae families. In preferred embodiments, the said plant or part thereof to which the endophyte is capable of conferring a PGP phenotype is a plant of the Fabaceae family, preferably a plant of a Glycine spp. (e.g., G. max, G. clandestina, G. tomentella, G. tabacina, G. canescens, G. latrobeana, G. microphylla, and/or G. soja), and/or a plant of the or Poaceae family, preferably a plant of the Triticum spp. (e.g., T. aestivum, T. durum, T. spelta, T. dicoccon, T. dicoccoides, T. turgidum, T. polonicum, T. carthlicum, T. turanicum, T. timopheevii, T. monococcum, and/or T. boeotictim).

In preferred embodiments, the Curtobacterium sp. endophyte includes a nucleic acid sequence encoding a 16S fourth hypervariable (V4) region of an rRNA gene including sequences shown in FIGS. 3 to 8, hereto (SEQ ID NOs: 1, 2, 3, 4, 5 and 6), or a sequence having at least approximately 95%, more preferably 97%, more preferably 98%, more preferably 99% sequence identity to the full length of any one of SEQ ID NOs: 1, 2, 3, 4, 5 or 6.

By “nucleic acid” as used herein, we mean a chain of nucleotides encoding genetic information. The term generally refers to genes or functionally active fragments or variants thereof and or other sequences in the genome of the organism that influence its properties. The term ‘nucleic acid’ includes DNA (such as cDNA or genomic DNA) and RNA (such as mRNA or microRNA) that is single- or double-stranded, optionally containing synthetic, non-natural or altered nucleotide bases, synthetic nucleic acids, and combinations thereof.

Nucleic acids according to the invention may be full-length genes or a part thereof and are also referred to as “nucleic acid fragments” and “nucleotide sequences” in this specification.

For convenience, the expression “nucleic acid or nucleic acid fragment” is used to cover all of these.

The present invention encompasses variants of the nucleic acids of the present invention. Such variants include naturally occurring allelic variants and non-naturally occurring variants. Additions, deletions, substitutions and derivatisations of one or more of the nucleotides are contemplated so long as the modifications do not result in loss of functional activity of the variant.

Preferably the variant has at least approximately 95% sequence identity to the relevant part of the nucleic to which or variant corresponds, more preferably at least approximately 97% identity, even more preferably at least approximately 98% identity, most preferably at least approximately 99% identity. Such functionally active variants include, for example, those having conservative nucleic acid changes.

Also provided is a composition comprising an endophyte as described herein together with a suitable carrier. For example, the composition may be a composition suitable for inoculating or infecting a seed, plant or plant part or may be suitable for inoculating soil prior to planting. The carrier may be any carrier that is not detrimental to the endophyte and may be solid or liquid. For example, in some embodiments the carrier comprises water. In some embodiments, the endophyte may be in a latent state, for example, it may be cryopreserved or lyophilised. The composition may also include components that facilitate the viability of the endophytes in the composition. For example, the composition may comprise proteins and/or carbohydrates/sugars that facilitate viability of the endophyte. Suitable proteins include milk proteins and suitable carbohydrates/sugars include maltose. In some embodiments, the composition may include components that assist with inoculation of the seed, plant or plant part, or transportation or storage of the compositions. The compositions may further contain components such as a plant growth regulator, encapsulation agent, wetting agent or dispersing agent to enhance the effect of the composition. The endophyte may be absorbed onto a granulated carrier that may be planted with a seed or applied to the soil at the time of planting.

In some embodiments, the composition is a fermentation broth that is capable of supporting the growth of the endophyte, such as Lysogeny broth or Nutrient broth. These broths may for example include nutrients as supplied by components such as tryptone (5-15 g/L), peptone (3-15 g/L) yeast extract (2-10 g/L), beef extract (2-10 g/L), sodium chloride (50 g/L for 5% to 200 g/L for 20% and all amounts in between). The broths are aqueous and contain water making up to the required volume.

In some embodiments, the endophyte may be isolated from the fermentation broth, for example by centrifuge, to isolate the pellet of endophyte for resuspension in a suitable buffer, such as phosphate buffered saline. A suitable buffer pH range is 7 to 8, especially about 7.4.

Also provided is a seed or embryo coated with a composition comprising the endophyte described herein. Plant seeds or embryos isolated from plant seeds may be coated with one or more endophytes as disclosed herein in a solid or liquid suspension, directly or in combination with a suitable carrier, binder and/or filler. Suitable carriers, binders and fillers include peat, lime, biochar, chitosan, methyl cellulose, carboxymethylcellulose, gum arabic, polysaccharide Pelgel®, xanthan gum and alginate. The identity of the carrier, binder and/or filler may depend on the endophyte used and the seed being treated. The coating may be a seed dressing, a film, a pellet or an encrustation.

In one embodiment, the plant seed or embryo may be treated with an aqueous composition comprising alginate and the endophyte followed by treatment with a complexing agent. For example, the endophyte may be in a composition comprising sodium alginate and after coating the seed with sufficient composition, a complexing agent such as calcium chloride may be added so solidify the alginate polymer thereby coating the seed. The sodium alginate may be present in the composition at a concentration of 1 to about 10% w/volume in water, especially 3 to 5% w/v. The calcium chloride solution may be at any concentration suitable to result in polymerisation of the alginate, for example, 1-1000 mM, 20-500 mM or 50-300 mM. The thickness of the seed coating once solidified may be in the range of about 0.1 to about 5 mm, especially about 0.25 to about 1.5 mm. In some embodiments, a second coating may be applied to the seed or embryo, this outer coating not including the endophyte.

In another aspect, the present invention provides a seed, plant, or part thereof inoculated with one or more Curtobacterium sp. endophytes as herein described.

The endophytes of the present invention may have the ability to be transferred through propagative material from one plant generation to the next. The endophyte may then spread or locate to other tissues as the plant grows, for example, to roots. Alternatively, or in addition, the endophyte may be recruited to the plant root, for example from soil, and spread or locate to other tissues. In either sense, the endophyte may be said to be stably inoculated or infected to the plant.

Therefore, the present invention also provides a seed, plant, plant propagative material, or other plant part derived from a plant inoculated with an endophyte as herein described and infected therewith.

The present invention provides the use of an endophyte as described herein to produce a seed, plant, or part thereof infected, preferably stably infected, with said one or more of the endophytes.

The present invention also provides a method for conferring a PGP phenotype to a seed, plant, or part thereof, the method including inoculating to the seed, plant, or part thereof an endophyte as herein described. In all preferred embodiments, the seed, plant, or plant part inoculated or otherwise infected with an endophyte as described herein will exhibit an endophyte-conferred PGP phenotype, or in other words, the endophyte will confer thereto a plant growth promoting endophyte.

In a preferred embodiment of this aspect of the present invention, the plant or part thereof may be free of said endophyte prior to inoculation and may be stably infected with said endophyte.

Plants are often associated with many endophyte species and strains with varying functions and properties. The present invention also provides an efficient method for selecting a plant growth promoting endophyte, in particular a plant growth promoting endophyte of a plant of the Glycine genus.

Thus, in another aspect, the present invention provides a method for selecting a plant growth promoting endophyte of a seed or plant of the Glycine genus, wherein the method includes:
a. substantially purifying or isolating one or more endophytes;
b. subjecting the endophyte(s) to an in vitro plant growth promotion assay and/or an in planta plant growth promotion assay; and
   based thereon, selecting an endophyte which is capable of conferring a plant growth promotion phenotype to the seed or plant from which it is substantially purified or isolated and/or is capable of conferring a plant growth promotion phenotype to a seed, plant, or part thereof to which it is inoculated.

In this aspect, the plant of the Glycine family, the PGP gene encoding one or more proteins that facilitate the uptake and/or utilisation of water and/or nutrients, and the seed, plant, or part thereof to which the endophyte is capable of conferring a PGP phenotype may be as herein described. The skilled worker will further be familiar with techniques used to perform in vitro PGP activity assays and in planta PGP activity assays. As such, PGP activity assays may detect and/or quantify water and/or nutrient uptake/utilisation by the plant inoculated with the endophyte. The skilled worker will also be familiar with methods for substantially purifying or isolating endophytes, which generally includes:
a. providing one or more samples of the seed, plant, or part thereof;
b. preparing an extract(s) from the sample(s); and
c. growing endophyte colonies from the extract(s),
   the colonies generally representing purified or isolated endophytes. In a preferred embodiment of this aspect of the present invention, the step of substantially purifying or isolating one or more endophytes may include providing one or more samples of the seed, plant, or part thereof, preparing an extract(s) from the sample(s), and growing bacterial colonies from the extract(s).

In a preferred embodiment, the sample of plant material may be selected from one or more of the group consisting of flowers, flower bracts, leaves, petioles, seeds, seedpods, roots and stem.

Preferably, the endophytes are substantially purified or isolated from association with the seeds or seedpods of a plant of the Glycine genus.

In preferred embodiments, said method further includes the step of subjecting said selected endophyte(s) to genetic analysis to identify the endophyte species, and preferably the selected endophyte is a Curtobacterium sp. endophyte as herein described.

In this specification, the term ‘comprises’ and its variants are not intended to exclude the presence of other integers, components or steps.

In this specification, reference to any prior art in the specification is not and should not be taken as an acknowledgement or any form of suggestion that this prior art forms part of the common general knowledge in Australia or any other jurisdiction or that this prior art could reasonably expected to be combined by a person skilled in the art.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE FIGURES

FIG. 1 depicts alpha and beta diversity analyses of the seed microbiomes of Glycine plant species. (A) Significant differences between Glycine clandestina and Glycine max plant species, according to the Shannon diversity index, are indicated by the lower-case letters ‘a’ and ‘b’ in the box-and-whiskers plots (Kruskal Wallis pairwise test; p≤0.05). (B) Community clustering of bacterial compositions, and significant differences therein (ANOSIM pairwise test; p≤0.05), as determined by the two dimensional unweighted-Unifrac distances matrix are shown in the principal coordinate analysis (PCoA) plot.

FIG. 2 depicts Venn diagrams representing the shared and unique bacterial OTUs associated with G. clandestina and G. max seeds. Shared OTUs are those present in the intersection of the two circles, while the OTUs unique to G. clandestina and G. max are present in the periphery of the left and right circles, respectively

FIG. 3 depicts the 16S rRNA sequence of bacterial species P3-Gcland-NS-Dand-IS-96-1 (SEQ ID NO: 1).

FIG. 4 depicts the 16S rRNA sequence of bacterial species P3-Gcland-NS-Dand-IS-26-1 (SEQ ID NO: 2).

FIG. 5 depicts the 16S rRNA sequence of bacterial species P2-Gtab-NS-kalkallo-IS-65-1 (SEQ ID NO: 3).

FIG. 6 depicts the 16S rRNA sequence of bacterial strain P2-Gtab-NS-kalkallo-IS-64-1 (SEQ ID NO: 4).

FIG. 7 depicts the 16S rRNA sequence of bacterial species P3-Gtab-NS-kalkallo-IS-74-1 (SEQ ID NO: 5).

FIG. 8 depicts the 16S rRNA sequence of bacterial species P3-Gcland-NS-Dand-IS-27-1 (SEQ ID NO: 6).

FIG. 9 depicts the phylogeny of Curtobacterium spp. and Curtobacterium species (P3-Gcland-NS-Dand-IS-96-1, P3-Gcland-NS-Dand-IS-26-1, P2-Gtab-NS-kalkallo-IS-65-1, P2-Gtab-NS-kalkallo-IS-64-1, P3-Gtab-NS-kalkallo-IS-74-1 and P3-Gcland-NS-Dand-IS-27-1), where the maximum-likelihood tree was inferred from the 36 genes conserved among 14 genomes. Local support values are shown next to the branches (Shimodaira-Hasegawa test using 1000 resamples).

FIG. 10 depicts the PGP gene associated with UDP-glucose 6-dehydrogenase (ugd) identified in the annotated genome of Curtobacterium flaccumfaciens strain CFBP3419 (SEQ ID NO: 7).

FIG. 11 depicts the PGP gene associated with UDP-glucose 6-dehydrogenase (ugd) identified in the annotated genome of bacterial species P3-Gcland-NS-Dand-IS-96-1 (SEQ ID NO: 8).

FIG. 12 depicts the PGP gene associated with UDP-glucose 6-dehydrogenase (ugd) identified in the annotated genome of bacterial species P3-Gcland-NS-Dand-IS-26-1 (SEQ ID NO: 9).

FIG. 13 depicts the PGP gene associated with UDP-glucose 6-dehydrogenase (ugd) identified in the annotated genome of bacterial species P2-Gtab-NS-kalkallo-IS-65-1 (SEQ ID NO: 10).

FIG. 14 depicts the PGP gene associated with UDP-glucose 6-dehydrogenase (ugd) identified in the annotated genome of bacterial strain P2-Gtab-NS-kalkallo-IS-64-1 (SEQ ID NO: 11).

FIG. 15 depicts the PGP gene associated with UDP-glucose 6-dehydrogenase (ugd) identified in the annotated genome of bacterial species P3-Gtab-NS-kalkallo-IS-74-1 (SEQ ID NO: 12).

FIG. 16 depicts the PGP gene associated with UDP-glucose 6-dehydrogenase (ugd) identified in the annotated genome of bacterial species P3-Gcland-NS-Dand-IS-27-1 (SEQ ID NO: 13).

FIG. 17 depicts the PGP gene associated with glucose-1-dehydrogenase (gcd) identified in the annotated genome of bacterial species P3-Gcland-NS-Dand-IS-96-1 (SEQ ID NO: 14).

FIG. 18 depicts the PGP gene associated with glucose-1-dehydrogenase (gcd) identified in the annotated genome of bacterial species P3-Gcland-NS-Dand-IS-26-1 (SEQ ID NO: 15).

FIG. 19 depicts the PGP gene associated with glucose-1-dehydrogenase (gcd) identified in the annotated genome of bacterial species P3-Gtab-NS-kalkallo-IS-74-1 (SEQ ID NO: 16).

FIG. 20 depicts the PGP gene associated with a phosphonatase (phnB) identified in the annotated genome of bacterial species P3-Gcland-NS-Dand-IS-96-1 (SEQ ID NO: 17).

FIG. 21 depicts the PGP gene associated with a phosphonatase (phnB) identified in the annotated genome of bacterial species P3-Gcland-NS-Dand-IS-26-1 (SEQ ID NO: 18).

FIG. 22 depicts the PGP gene associated with a phosphonatase (phnC) identified in the annotated genome of Curtobacterium flaccumfaciens strain CFBP3419 (SEQ ID NO: 19).

FIG. 23 depicts the PGP gene associated with a phosphonatase (phnC) identified in the annotated genome of bacterial species P3-Gcland-NS-Dand-IS-96-1 (SEQ ID NO: 20).

FIG. 24 depicts the PGP gene associated with a phosphonatase (phnC) identified in the annotated genome of bacterial species P3-Gcland-NS-Dand-IS-26-1 (SEQ ID NO: 21).

FIG. 25 depicts the PGP gene associated with a phosphonatase (phnC) identified in the annotated genome of bacterial strain P2-Gtab-NS-kalkallo-IS-64-1 (SEQ ID NO: 22).

FIG. 26 depicts the PGP gene associated with a phosphonatase (phnC) identified in the annotated genome of bacterial species P3-Gcland-NS-Dand-IS-27-1 (SEQ ID NO: 23).

FIG. 27 depicts the PGP gene associated with a phosphonatase (phnD) identified in the annotated genome of Curtobacterium flaccumfaciens strain CFBP3419 (SEQ ID NO: 24).

FIG. 28 depicts the PGP gene associated with a phosphonatase (phnD) identified in the annotated genome of bacterial species P3-Gcland-NS-Dand-IS-96-1 (SEQ ID NO: 25).

FIG. 29 depicts the PGP gene associated with a phosphonatase (phnD) identified in the annotated genome of bacterial species P3-Gcland-NS-Dand-IS-26-1 (SEQ ID NO: 26).

FIG. 30 depicts the PGP gene associated with a phosphonatase (phnD) identified in the annotated genome of bacterial species P2-Gtab-NS-kalkallo-IS-64-1 (SEQ ID NO: 27).

FIG. 31 depicts the PGP gene associated with a phosphonatase (phnD) identified in the annotated genome of bacterial species P3-Gcland-NS-Dand-IS-27-1 (SEQ ID NO: 28).

FIG. 32 depicts the PGP gene associated with a phosphonatase (phnE) identified in the annotated genome of Curtobacterium flaccumfaciens strain CFBP3419 (SEQ ID NO: 29).

FIG. 33 depicts the PGP gene associated with a phosphonatase (phnE) identified in the annotated genome of bacterial species P3-Gcland-NS-Dand-IS-96-1 (SEQ ID NO: 30).

FIG. 34 depicts the PGP gene associated with a phosphonatase (phnE) identified in the annotated genome of bacterial species P3-Gcland-NS-Dand-IS-26-1 (SEQ ID NO: 31).

FIG. 35 depicts the PGP gene associated with a phosphonatase (phnE) identified in the annotated genome of bacterial strain P2-Gtab-NS-kalkallo-IS-64-1 (SEQ ID NO: 32).

FIG. 36 depicts the PGP gene associated with a phosphonatase (phnE) identified in the annotated genome of bacterial species P3-Gcland-NS-Dand-IS-27-1 (SEQ ID NO: 33).

FIG. 37 depicts the PGP gene associated with a phosphate transporter (pstS) identified in the annotated genome of Curtobacterium flaccumfaciens strain CFBP3419 PBP (SEQ ID NO: 34).

FIG. 38 depicts the PGP gene associated with a phosphate transporter (pstS) identified in the annotated genome of bacterial species P3-Gcland-NS-Dand-IS-96-1 (SEQ ID NO: 35).

FIG. 39 depicts the PGP gene associated with a phosphate transporter (pstS) identified in the annotated genome of bacterial species P3-Gcland-NS-Dand-IS-26-1 (SEQ ID NO: 36).

FIG. 40 depicts the PGP gene associated with a phosphate transporter (pstS) identified in the annotated genome of bacterial species P2-Gtab-NS-kalkallo-IS-65-1 (SEQ ID NO: 37).

FIG. 41 depicts the PGP gene associated with a phosphate transporter (pstS) identified in the annotated genome of bacterial strain P2-Gtab-NS-kalkallo-IS-64-1 (SEQ ID NO: 38).

FIG. 42 depicts the PGP gene associated with a phosphate transporter (pstS) identified in the annotated genome of bacterial species P3-Gtab-NS-kalkallo-IS-74-1 (SEQ ID NO: 39).

FIG. 43 depicts the PGP gene associated with a phosphate transporter (pstS) identified in the annotated genome of bacterial species P3-Gcland-NS-Dand-IS-27-1 (SEQ ID NO: 40).

FIG. 44 depicts the PGP gene associated with a phosphate transporter (pstA) identified in the annotated genome of Curtobacterium flaccumfaciens strain CFBP3419 (SEQ ID NO: 41).

FIG. 45 depicts the PGP gene associated with a phosphate transporter (pstA) identified in the annotated genome of bacterial species P3-Gcland-NS-Dand-IS-96-1 (SEQ ID NO: 42).

FIG. 46 depicts the PGP gene associated with a phosphate transporter (pstA) identified in the annotated genome of bacterial species P3-Gcland-NS-Dand-IS-26-1 (SEQ ID NO: 43).

FIG. 47 depicts the PGP gene associated with a phosphate transporter (pstA) identified in the annotated genome of bacterial species P2-Gtab-NS-kalkallo-IS-65-1 (SEQ ID NO: 44).

FIG. 48 depicts the PGP gene associated with a phosphate transporter (pstA) identified in the annotated genome of bacterial strain P2-Gtab-NS-kalkallo-IS-64-1 (SEQ ID NO: 45).

FIG. 49 depicts the PGP gene associated with a phosphate transporter (pstA) identified in the annotated genome of bacterial species P3-Gtab-NS-kalkallo-IS-74-1 (SEQ ID NO: 46).

FIG. 50 depicts the PGP gene associated with a phosphate transporter (pstA) identified in the annotated genome of bacterial species P3-Gcland-NS-Dand-IS-27-1 (SEQ ID NO: 47).

FIG. 51 depicts the PGP gene associated with a phosphate transporter (pstB) identified in the annotated genome of Curtobacterium flaccumfaciens strain CFBP3419 (SEQ ID NO: 48).

FIG. 52 depicts the PGP gene associated with a phosphate transporter (pstB) identified in the annotated genome of bacterial species P3-Gcland-NS-Dand-IS-96-1 (SEQ ID NO: 49).

FIG. 53 depicts the PGP gene associated with a phosphate transporter (pstB) identified in the annotated genome of bacterial species P3-Gcland-NS-Dand-IS-26-1 (SEQ ID NO: 50).

FIG. 54 depicts the PGP gene associated with a phosphate transporter (pstB) identified in the annotated genome of bacterial species P2-Gtab-NS-kalkallo-IS-65-1 (SEQ ID NO: 51).

FIG. 55 depicts the PGP gene associated with a phosphate transporter (pstB) identified in the annotated genome of bacterial strain P2-Gtab-NS-kalkallo-IS-64-1 (SEQ ID NO: 52).

FIG. 56 depicts the PGP gene associated with a phosphate transporter (pstB) identified in the annotated genome of bacterial species P3-Gtab-NS-kalkallo-IS-74-1 (SEQ ID NO: 53).

FIG. 57 depicts the PGP gene associated with a phosphate transporter (pstB) identified in the annotated genome of bacterial species P3-Gcland-NS-Dand-IS-27-1 (SEQ ID NO: 54).

FIG. 58 depicts the PGP gene associated with a phosphate transporter (pstC) identified in the annotated genome of Curtobacterium flaccumfaciens strain CFBP3419 (SEQ ID NO: 55).

FIG. 59 depicts the PGP gene associated with a phosphate transporter (pstC) identified in the annotated genome of bacterial species P3-Gcland-NS-Dand-IS-96-1 (SEQ ID NO: 56).

FIG. 60 depicts the PGP gene associated with a phosphate transporter (pstC) identified in the annotated genome of bacterial species P3-Gcland-NS-Dand-IS-26-1 (SEQ ID NO: 57).

FIG. 61 depicts the PGP gene associated with a phosphate transporter (pstC) identified in the annotated genome of bacterial species P2-Gtab-NS-kalkallo-IS-65-1 (SEQ ID NO: 58).

FIG. 62 depicts the PGP gene associated with a phosphate transporter (pstC) identified in the annotated genome of bacterial strain P2-Gtab-NS-kalkallo-IS-64-1 (SEQ ID NO: 59).

FIG. 63 depicts the PGP gene associated with a phosphate transporter (pstC) identified in the annotated genome of bacterial species P3-Gtab-NS-kalkallo-IS-74-1 (SEQ ID NO: 60).

FIG. 64 depicts the PGP gene associated with a phosphate transporter (pstC) identified in the annotated genome of bacterial species P3-Gcland-NS-Dand-IS-27-1 (SEQ ID NO: 61).

FIG. 65 depicts the PGP gene associated with a phosphate transporter (phoR) identified in the annotated genome of Curtobacterium flaccumfaciens strain CFBP3419 (SEQ ID NO: 62).

FIG. 66 depicts the PGP gene associated with a phosphate transporter (phoR) identified in the annotated genome of bacterial species P2-Gtab-NS-kalkallo-IS-65-1 (SEQ ID NO: 63).

FIG. 67 depicts the PGP gene associated with a phosphate transporter (phoR) identified in the annotated genome of bacterial species P3-Gtab-NS-kalkallo-IS-74-1 (SEQ ID NO: 64).

FIG. 68 depicts the PGP gene associated with a phosphate transporter (phoR) identified in the annotated genome of bacterial species P3-Gcland-NS-Dand-IS-27-1 (SEQ ID NO: 65).

FIG. 69 depicts the PGP gene associated with a phosphate transporter (phoP) identified in the annotated genome of bacterial species P2-Gtab-NS-kalkallo-IS-65-1 (SEQ ID NO: 66).

FIG. 70 depicts the PGP gene associated with a phosphate transporter (phoP) identified in the annotated genome of bacterial strain P2-Gtab-NS-kalkallo-IS-64-1 (SEQ ID NO: 67).

FIG. 71 depicts the PGP gene associated with a phosphate transporter (phoP) identified in the annotated genome of bacterial species P3-Gtab-NS-kalkallo-IS-74-1 (SEQ ID NO: 68).

FIG. 72 contains photographs of Curtobacterium spp. (P3-Gcland-NS-Dand-IS-96-1, P3-Gcland-NS-Dand-IS-26-1, P2-Gtab-NS-kalkallo-IS-65-1 and P2-Gtab-NS-kalkallo-IS-64-1) in vitro Pikovskayas agar assays and provides pictorial representation of phosphate solubilisation through the establishment of clear zones around the bacterial colonies (i.e., areas where phosphate is solubilised from the surrounding media).

FIG. 73 contains photographs of Glycine max used in in planta Pikovskayas agar assays and provides a pictorial representation of the plant growth promoting phenotype corresponding to enhanced phosphate solubilisation in Glycine max inoculated with Curtobacterium spp. (P3-Gcland-NS-Dand-IS-96-1, P3-Gcland-NS-Dand-IS-26-1, and P2-Gtab-NS-kalkallo-IS-64-1) relative to control plants.

FIG. 74 depicts the difference in shoot length of Glycine max control seedlings and seedlings inoculated with Curtobacterium spp. (P3-Gcland-NS-Dand-IS-96-1, P3-Gcland-NS-Dand-IS-26-1 and P2-Gtab-NS-kalkallo-IS-64-1) grown on Pikovskayas agar. Statistically significant differences are denoted using a star (one-way ANOVA and Tukey test; p≤0.05).

FIG. 75 depicts the difference in root length of Glycine max control seedlings and seedlings inoculated with Curtobacterium spp. (P3-Gcland-NS-Dand-IS-96-1, P3-Gcland-NS-Dand-IS-26-1 and P2-Gtab-NS-kalkallo-IS-64-1) grown on Pikovskayas agar. Statistically significant differences are denoted using a star (one-way ANOVA and Tukey test; p≤0.05).

FIG. 76 depicts the difference in root length of Glycine max control seedlings and seedlings inoculated with Curtobacterium spp. (P3-Gcland-NS-Dand-IS-96-1, P3-Gcland-NS-Dand-IS-26-1 and P2-Gtab-NS-kalkallo-IS-64-1) grown on filter paper. Statistically significant differences are denoted using a star (one-way ANOVA and Tukey test; p≤0.05).

FIG. 77 depicts the difference in shoot length of Glycine max negative control, positive control, and test seedlings grown in a greenhouse for 3 weeks. Positive control and test seedlings were inoculated with Curtobacterium flaccumfaciens strain (D3-25) and Curtobacterium sp. P3-Gcland-NS-Dand-IS-96-1, respectively. Statistically significant differences are denoted using a star (one-way ANOVA and Tukey test; p≤0.05).

FIG. 78 depicts the difference in root length of Glycine max negative control, positive control, and test seedlings grown in a greenhouse for 3 weeks. Positive control and test seedlings were inoculated with Curtobacterium flaccumfaciens strain (D3-25) and Curtobacterium sp. P3-Gcland-NS-Dand-IS-96-1, respectively. Statistically significant differences are denoted using a star (one-way ANOVA and Tukey test; p≤0.05).

FIG. 79 depicts the difference in shoot length of Triticum sp. (cultivar Bob White Red Haplotype) negative control, positive control, and test seedlings grown in a greenhouse for 3 weeks. Positive control seedlings were inoculated with Curtobacterium flaccumfaciens strain (D3-25), and test seedlings were inoculated with Curtobacterium sp. P3-Gcland-NS-Dand-IS-96-1 or P2-Gtab-NS-kalkallo-IS-64-1. Statistically significant differences are denoted using a star (one-way ANOVA and Tukey test; p≤0.05).

FIG. 80 depicts the shoot lengths of Glycine max negative control (C mean), positive control (D3-25 Mean), and test seedlings (96 Mean and 64 Mean) grown as potted plants and subjected to well-watered conditions (300 mL per pot every 48 hr) for 4 weeks. Positive control seedlings were inoculated with Curtobacterium flaccumfaciens strain (D3-25), and test seedlings were inoculated with Curtobacterium sp. P3-Gcland-NS-Dand-IS-96-1 or P2-Gtab-NS-kalkallo-IS-64-1. Statistically significant differences are denoted using a star (one-way ANOVA and Tukey test; p≤0.05).

FIG. 81 depicts the shoot lengths of Glycine max negative control (C mean), positive control (D3-25 Mean), and test seedlings (96 Mean and 64 Mean) grown as potted plants and subjected to mild drought conditions (150 mL per pot every 48 hr) for 4 weeks. Positive control seedlings were inoculated with Curtobacterium flaccumfaciens strain (D3-25), and test seedlings were inoculated with Curtobacterium sp. P3-Gcland-NS-Dand-IS-96-1 or P2-Gtab-NS-kalkallo-IS-64-1. Statistically significant differences are denoted using a star (one-way ANOVA and Tukey test; p≤0.05).

FIG. 82 depicts the shoot lengths of Glycine max negative control (C mean), positive control (D3-25 Mean), and test seedlings (96 Mean and 64 Mean) grown as potted plants and subjected to severe drought conditions (50 mL per pot every 48 hr) for 4 weeks. Positive control seedlings were inoculated with Curtobacterium flaccumfaciens strain (D3-25), and test seedlings were inoculated with Curtobacterium sp. P3-Gcland-NS-Dand-IS-96-1 or P2-Gtab-NS-kalkallo-IS-64-1. Statistically significant differences are denoted using a star (one-way ANOVA and Tukey test; p≤0.05).

### DETAILED DESCRIPTION OF EMBODIMENTS

In the following examples it is demonstrated that five novel Curtobacterium bacterial species P3-Gcland-NS-Dand-IS-96-1, P3-Gcland-NS-Dand-IS-26-1, P2-Gtab-NS-Kalkallo-IS-65-1, P3-Gcland-NS-Dand-IS-27-1, P3-Gtab-NS-Kalkallo-IS-74-1, and one novel Curtobacterium strain, P2-Gtab-NS-Kalkallo-IS-64-1, were isolated from soybean (Glycine spp.) plants. Each isolate displays a plant growth promotion phenotype, particularly phosphate solubilisation and drought tolerance, in in vitro and in planta assays. The genomes of the bacterial isolates have been sequenced and are shown to be related to one of Curtobacterium sp. strains MMLR14_010 MMLR14_010, ER1/G ER1/6, MCBA15_008 MCBA15_008, or MCBD17_032 MCBD17_032.

### Example 1—Profiling Glycine spp. Microbiome

The seed microbiome of Glycine Crop Wild Relatives (CWR) was compared to commercial Glycine max to determine similarities across the species. The core microbiome of G. clandestina was identified and compared to G. max to identify operational taxonomic units (OTUs) present in (Scenario I) both Glycine CWRs and G. max in order to identify strains that could be transferred from one species to another and which could potentially offer enhanced bioactivity, or (Scenario II) to identify OTUs present only in Glycine CWRs in order to identify strains that could be introduced into G. max and which could potentially offer novel bioactivity. The process was validated by isolating strains from Glycine CWRs and G. max which were taxonomically identified and compared using MALDI-TOF MS to determine if there were strains that clustered according to Scenario I or Scenario II. Candidate microbes were identified that fulfilled both Scenarios and were also within the core microbiome of G. clandestina.

### Profiling the Glycine Seed Microbiome

Seedpods of G. clandestina were collected from six different Seed Accessions across greater Melbourne, Victoria, Australia and identified using the online database of “The Atlas of Living Australia” (https://www.ala.org.au). There was a minimum 15 km distance between each location, and pods were collected from an individual plant in the identified area. Seed for the Australian grown soybean cultivar (G. max) was obtained from NSW (3 accessions) and QLD (2 accession), Australia. All seeds were stored at approximately 20° C.

The seeds were washed ten times in sterile distilled water (SDW) and plated onto sterile filter paper (×3) soaked in SDW in sterile petri dishes. These plates were stored at room temperature in the dark to allow seedlings to germinate for up to 2 weeks. Once the seedlings were of sufficient size, a total of 16 seedlings were harvested per Glycine species and per location. DNA extraction of seedlings was performed in 96-well plates using the QIAGEN MagAttract 96 DNA Plant Core Kit according to manufacturers' instructions with minor modifications for use with a Biomek FX liquid handling station. The bacterial microbiome was profiled targeting the V4 region (515F and 806R) of the 16S rRNA gene according to the Illumina 16S Metagenomic Sequencing Library Preparation protocol, with minor modifications to include the use of PNA PCR blockers to reduce amplification of 16S rRNA genes sequences derived from the plant chloroplast genome and mitochondrial genome (Wagner et al. 2016). Paired-end sequencing was performed on HiSeq3000 using a 2×150 bp v3 chemistry cartridge. Sequence data was trimmed and merged using PandaSEQ (removal of low quality reads, 8 bp overlap of read 1 and read 2, removal of primers, final merged read length of 253 bp) (Massela et al. 2012). QIIME2 (release 2019.4) was used for dereplication for taxonomy assignment, removal of organelle OTUs, and statistical analysis (multivariate statistics for qualitative and quantitative OTU analysis; presence/absence searches for core microbiome analysis).

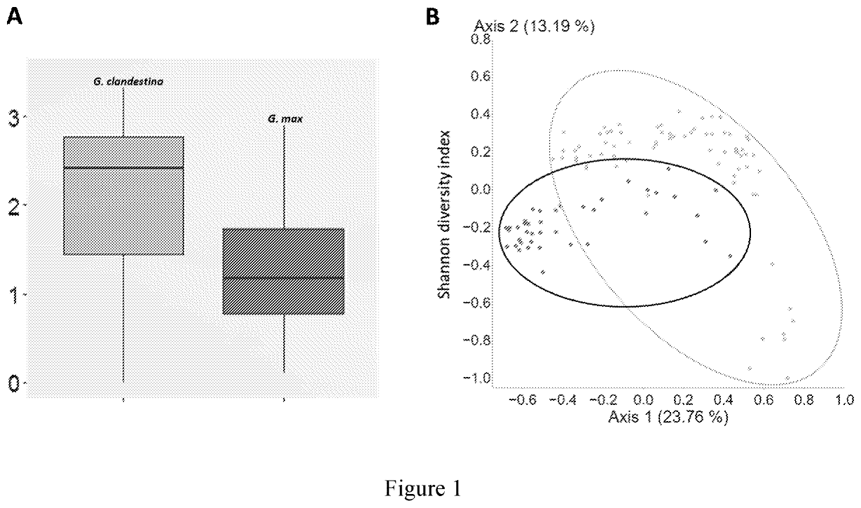

Seed microbiomes were assessed from G. clandestina accessions collected from 6 locations across the greater Melbourne region of Victoria and compared to G. max (five accessions). As shown in FIG. 1, the comparison utilised Unweighted UNIFRAC Distance Principal Components Analysis (i.e., a qualitative assessment that utilises OTU phylogeny) and Shannon diversity index (i.e., a quantitative measure of bacterial diversity within a community). The microbiomes of G. clandestina and G. max clustered separately except some replicates from G. max clustering together with that of G. clandestina, indicating the phylogeny of some OTUs from both species within the microbiomes were similar (see, i.e., FIG. 1B). When grouped based on “Plant Species”, bacterial diversity within the G. clandestina (2.4) microbiome was found to be significantly more diverse than within G. max (1.2) (p=0.000042) (see FIG. 1A).

An assessment of the core microbiome of G. clandestina identified 27 OTUs shared between G. clandestina and G. max, with these OTUs representing microbes that could potentially be transferred between the two species, including Pseudomonas sp., Pantoea sp., Delftia sp., Massilia sp., Enterobacteriaceae sp., Ralstonia sp., Burkholderiaceae sp., Acinetobacter sp., Herbaspirillum sp., Escherichia-Shigella sp., Stenotrophomonas sp., Burkholderia sp., Bacillus sp., Staphylococcus sp., Lactobacillus sp., Enterococcus sp., Carnobacterium sp., Sphingomonas sp., Methylobacterium sp., Bradyrhizobium sp., Novosphingobium sp., Curtobacterium sp., Micrococcaceae sp., Microbacterium sp., Rhodococcus sp., Hymenobacter sp., and Chryseobacterium sp. (see Table 1).

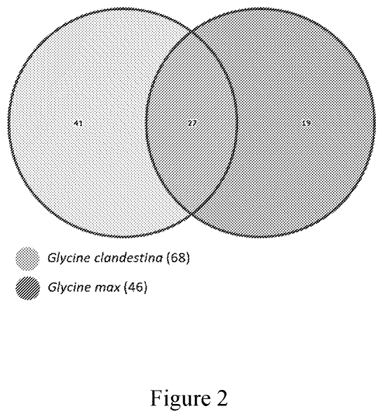

Additionally, there were 41 OTUs unique to G. clandestina and 19 OTUs unique to G. max that were also present in G. max (FIG. 2). Furthermore, there were 22 OTUs that were absent from G. max, with these OTUs representing microbes that could potentially be introduced into G. max.

TABLE 1
Relative abundance of the core bacterial OTUs associated
to G. clandestina and G. max seeds. Bacterial OTUs
with relative abundance >0.1% are highlighted in bold

| S. | | Core Bacterial | G. | G. |
|---|---|---|---|---|
| NO. | Class | ASVs | clandestina | max |
| 1 | Gammaproteobacte- | Pseudomonas | 44.604 | 47.884 |
| 2 | ria | Pantoea | 7.951 | 31.394 |
| 3 | | Delftia | 5.126 | 0.041 |
| 4 | | Massilia | 3.268 | 2.142 |
| 5 | | Enterobacteriaceae | 2.713 | 1.409 |
| 6 | | Ralstonia | 1.375 | 0.033 |
| 7 | | Burkholderiaceae | 1.175 | 0.172 |
| 8 | | Acinetobacter | 0.682 | 0.002 |
| 9 | | Herbaspirillum | 0.665 | 00.013 |
| 10 | | Escherichia-Shigella | 0.396 | 0.008 |
| 11 | | Stenotrophomonas | 0.183 | 0.420 |
| 12 | | Burkholderia | 0.052 | 0.158 |
| 13 | Bacilli | Bacillus | 3.136 | 3.808 |
| 14 | | Staphylococcus | 0.475 | 0.031 |
| 15 | | Lactobacillus | 0.384 | 0.017 |
| 16 | | Enterococcus | 0.360 | 0.006 |
| 17 | | Carnobacterium | 0.070 | 0.019 |
| 18 | Alphaproteobacteria | Sphingomonas | 13.861 | 3.509 |
| 19 | | Methylobacterium | 0.715 | 0.367 |
| 20 | | Bradyrhizobium | 0.086 | 0.008 |
| 21 | | Novosphingobium | 0.013 | 0.670 |
| 22 | Actinobacteria | Curtobacterium | 3.110 | 0.558 |
| 23 | | Micrococcaceae | 0.009 | 0.039 |
| 24 | | Microbacterium | 0.004 | 0.072 |
| 25 | | Rhodococcus | 0.004 | 0.002 |
| 26 | Flavobacteria | Hymenobacter | 3.759 | 0.059 |
| 27 | | Chryseobacterium | 0.005 | 0.667 |

### Example 2—Isolation and Characterisation of the Glycine Microbiome

Seed from the Glycine clandestina and G. max (1 accession) from Victoria and NSW respectively were washed and germinated as per Example 1. Seed was harvested by removing aerial tissue and root tissue and discarding the seed coat. The plant tissues were submerged in Phosphate Buffered Saline (PBS) to cover the plant tissue and ground using a Qiagen TissueLyser II (1 minute at 30 Hertz). A 10 μL aliquot of the macerate was added to 90 μL of PBS. Subsequent tenfold dilutions of the 1×10⁻¹ suspension were used to create additional 1×10⁻² to 1×10⁻⁴ suspensions. Once the suspensions were well mixed 20 μL aliquots of each suspension were plated onto Reasoners 2 Agar (R2A) for growth of bacteria. Dilutions that provided a good separation of bacterial colonies were subsequently used for isolation of individual bacterial colonies through re-streaking of single bacterial colonies from the dilution plates onto single R2A plates to establish a pure bacterial colony. A total of 117 microbial strains were isolated from sterile seedlings of six G. clandestina (n=85, 72% of total) seed accessions and one G. max (n=32, 28% of total) seed accession.

MALDI spectra were acquired for all bacterial and fungal strains to determine the relatedness of each strain. The analysis acquired and compared spectra of protein profiles from each bacterial and fungal strain using the Bruker MALDI Biotyper system. Single bacterial and fungal colonies of each strain were generated through streaking from glycerol stocks onto R2A plates and allowing colony growth for 48 hours. Single bacterial and fungal colonies were applied to a Bruker MALDI Biotyper target plate using the Extended Direct Transfer (EDT) method. In the EDT method bacterial and fungal strains were inoculated onto two consecutive wells on the target plate (primary spot and secondary spot), treated with 70% formic acid (for up to 30 mins) and covered with α-cyano-4-hydroxycinnamic acid (HCCA) matrix solution [10 mg HCAA in 1 mL of solvent solution: 50% volume μL ACN (acetonitrile), 47.5% volume μL water, and 2.5% volume μL TFA (trifluoroacetic acid)]. The plate was dried at room temperature. Escherichia coli strain ATCC 25922 was included as a quality control. The target plate was analysed in a Bruker MALDI-TOF ultrafleXtreme according to manufacturer's instructions. Protein spectra were calibrated with the Escherichia coli ATCC 25922 quality control strain and an internal standard.

Automated analysis of the raw spectral data was performed by the MALDI BioTyper automation 2.0 software (Bruker Daltonics) using default settings. Protein spectra were compared to MALDI BioTyper library (3,746 spectra—Jun. 9, 2010) for preliminary identification and taxonomical assignment. The raw protein spectra from each bacterial and fungal strain were processed through a data deconvolution workflow in the software Refiner, GeneData. The raw spectra from each plate (i.e., Batch) were processed separately, by first aligning the spectra to create a m/z grid (m/z×sample), then subtracting a baseline spectrum to reduce background noise across the grid, and finally aligning m/z across key reference spectra from the grid (e.g., E. coli ATCC 25922). Batches were then merged and processed further by first aligning m/z across key reference spectra (e.g., E. coli ATCC 25922) from the grid, followed by spectrum smoothing to reduce intensity jitter of putative peaks, then restricting m/z from a defined range, and detecting spectrum peaks using a resolution-based method, and finally filtering valid peaks by removing those that did not meet specific thresholds.

The resultant processed data of valid peaks and intensities was converted into a matrix for statistical analysis. The matrix was analysed in the software Analyst, Genedata. A Hierarchical Clustering analysis was conducted to compare protein spectra between novel bacterial and fungal strains. The analysis utilised the Positive Correlation (1-r) distance algorithm with complete linkage and only included values present in 50% of samples. A Hierarchical Clustering tree was generated whereby novel bacterial and fungal strains clustered based on similar protein profiles. The bacterial strains were identified as Pseudomonas, Pantoea, Sphingomonas, Methylobacterium, Curtoacterium, Bacillus, and Rhizobium. Some strains were not able to be identified. Molecular identification was then performed by selecting 36 strains representing different clades (identified and unidentified) from within the MALDI Hierarchical Clustering tree.

### Identification of Culturable Glycine Seed Microbiome

After quality filtering and assembly of the 36 strains, sequences were taxonomically classified in Kraken2 using a customised database. Based on Kraken2 classification, 27 of the 36 strains were linked to 8 bacterial genera, and 10 of the 36 strains were linked to 2 fungal genera. The bacterial strains were identified as Pseudomonas, Pantoea, Sphingomonas, Methylobacterium, Curtobacterium, Streptomyces, Bacillus and Chryseobacterium, while fungal strains were identified as Fusarium and Cryptococcus. When isolated bacterial sequences were mapped to 16S bacterial OTUs from the Glycine microbiome, 24.5% of the OTUs associated to G. max, and 19.2% OTUs from G. clandestina got a Blastn hit against an isolated bacteria with more than 96% similarity. The majority of the 16S bacterial OTUs in G. clandestina (80.8%) and G. max (75.5%) were found to be associated to the non-culturable microbiome (Table 2). Notably, all the bacterial genera mapped were also part of the core Glycine microbiome (see Table 1).

TABLE 2
Percentage of all seed-associated 16S RNA gene
ASVs that showed >96% similarity to at least
one Illumina ® sequence of the culturable bacteria

| | Percentage of 16S ASVs mapped with WGS data | |
|---|---|---|
| Isolate name | Glycine clandestina | Glycine max |
| Curtobacterium | 3 | 3 |
| Sphingomonas | 5 | 10 |
| Bacillus | 3 | 3 |
| Pantoea | 8 | 7 |
| Pseudomonas | 2 | 2 |
| Methylobacterium | 1 | 3 |
| Chryseobacterium | 1 | 1 |
| Others (Not mapped) | 97 | 89 |

### Example 3—Identification of Curtobacterium Spp. Bacterial Strains

Genomic DNA was extracted from overnight cultures using a Wizard® Genomic DNA Purification Kit (A1120, Promega, Madison, WI, USA). Genomic sequencing libraries (Illumina short reads) were prepared from the DNA using the PerkinElmer NEXTFLEX® Rapid XP DNA-Seq Kit (Cat #NOVA-5149-03) and sequenced on an Illumina NovaSeq 6000 platform. Genomic sequence data (raw reads) were assessed for quality and filtered to remove any adapter and index sequence and low-quality bases using fastp (Chen et al. 2018) with the following parameters: -w 8-3 -5. In addition, genomic libraries, sequencing, and quality control (MinION long reads) were prepared as per Example 1.

The whole genomes of bacterial strains were assembled with filtered long and short reads using Unicycler (Wick et al. 2017). Long reads were used for primary assembly and to resolve repeat regions in the genome, whereas short reads were used to correct small base-level errors. Assembly graphs were visualised using Bandage (Wick et al. 2015). Assembled genomes were taxonomically classified by Kraken2 (Wood & Salzberg 2014) using a custom database containing all completed bacterial reference genomes in NCBI (20/03/2020). The assembled genomes of bacterial strains were annotated using Prokka (Seemann, 2014) with a custom Curtobacterium protein database (based on Kraken2 classification) to predict genes and corresponding functions.

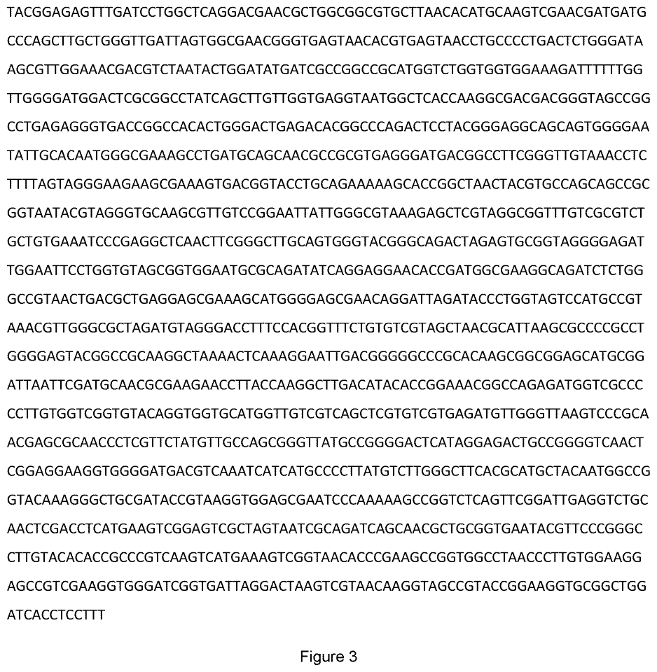

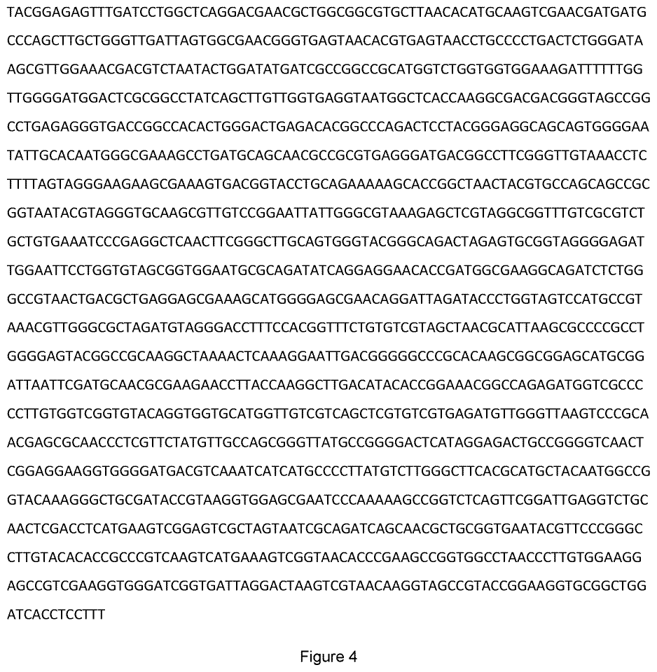

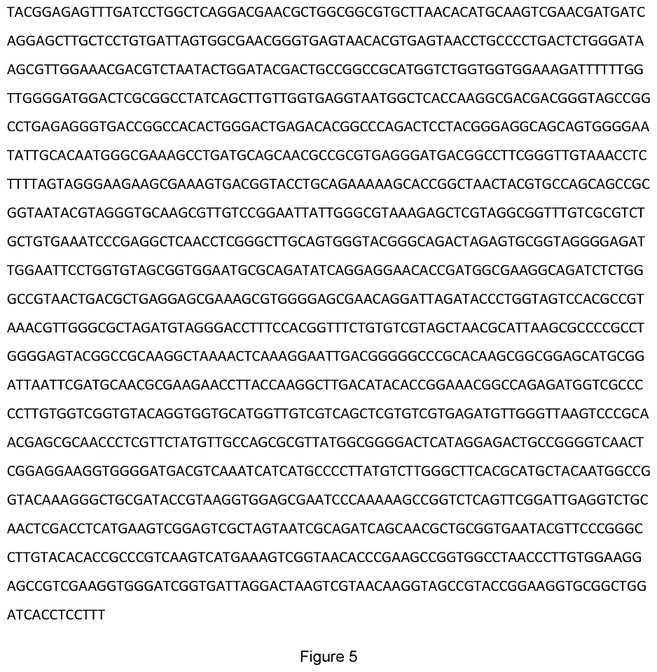

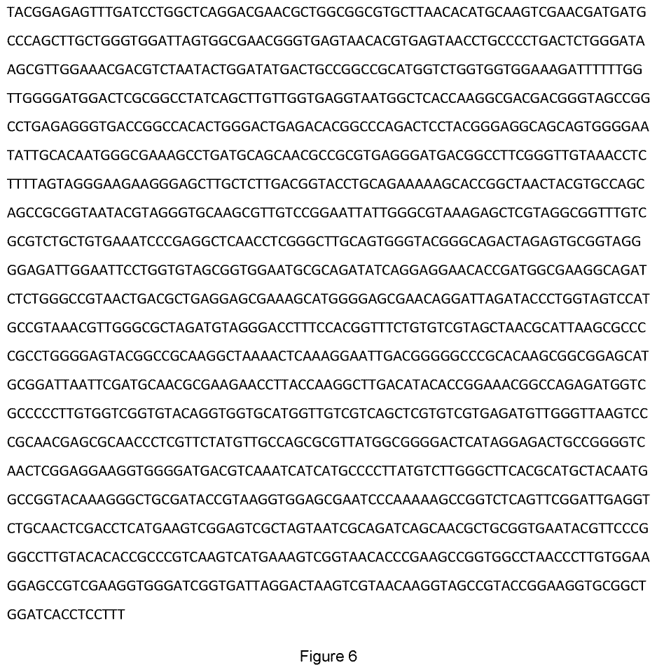

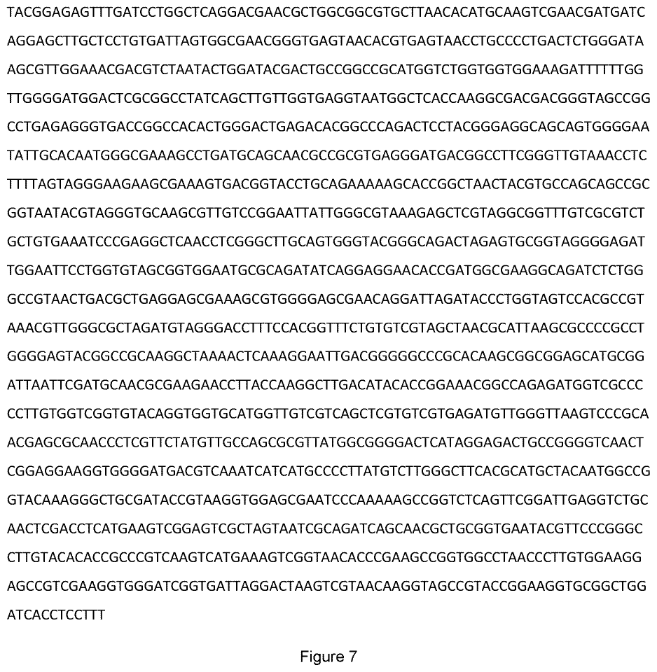

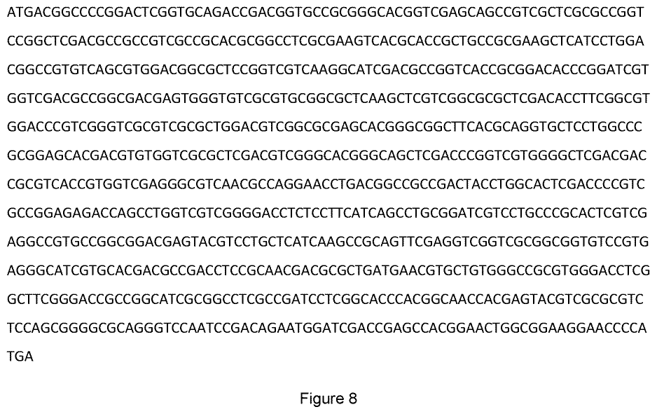

Of the core members of the microbiome, Curtobacterium was chosen as the genera of primary interest owing to a high number of putative plant growth promotion (PGP) genes highlighted by in silico analysis. The 16s rRNA sequences from the genomes of the sequenced isolates are shown in FIGS. 3 to 8 (i.e., SEQ ID NOs: 1-6). In addition, the strain names and corresponding SEQ ID NOs are shown in Table 3.

TABLE 3
Sequence listings of Curtobacterium spp. plant species

| Strain ID | SEQ ID NO |
|---|---|
| P3-Gcland-NS-Dand-IS-96-1 | 1 |
| P3-Gcland-NS-Dand-IS-26-1 | 2 |
| P2-Gtab-NS-Kalkallo-IS-65-1 | 3 |
| P2-Gtab-NS-Kalkallo-IS-64-1 | 4 |
| P3-Gtab-NS-Kalkallo-IS-74-1 | 5 |
| P3-Gcland-NS-Dand-IS-27-1 | 6 |

Comparative genomic analysis was performed on the Curtobacterium sp. against genome sequences of closely related strains available on NCBI. The average nucleotide identity (ANI) was calculated for the seven Curtobacterium isolates against 122 Curtobacterium sp. strains. The genome sequences were aligned and compared using minimap2 (Li, 2018). Each of the Curtobacterium isolates fell below the intraspecies boundary and above the interspecies boundary when compared to the plant pathogenic C. flaccumfaciens pv flaccumfaciens strain (CFBP3418), suggesting that the isolates belong to a different species/s (see Table 4). Based on the ANI, novel Curtobacterium species P3-Gcland-NS-Dand-IS-96-1, P3-Gcland-NS-Dand-IS-26-1, and P3-Gcland-NS-Dand-IS-27-1 were very closely related, with an ANI of 99.99% (Cluster C). P3-Gcland-NS-Dand-IS-96-1, P3-Gcland-NS-Dand-IS-26-1, and P3-Gcland-NS-Dand-IS-27-1 were most similar to an environmental Curtobacterium sp. MMLR14_010 strain (MMLR14_010) with an ANI of 93.01%, suggest that the isolates belong to a novel species (Chun et al. 2018; Richter & Rosselló-Móra 2009). Based on the ANI, novel Curtobacterium species P2-Gtab-NS-Kalkallo-IS-65-1 and P3-Gtab-NS-Kalkallo-IS-74-1 were very closely related, with an ANI of 99.99% (Müller et al., 2013) (Cluster A). P2-Gtab-NS-Kalkallo-IS-65-1 and P3-Gtab-NS-Kalkallo-IS-74-1 were most similar to an environmental Curtobacterium sp. ER1/6 strain (ER1/6) with an ANI of 89.88%, suggesting that the isolates belong to a novel species.

TABLE 4
ANI comparison of Curtobacterium spp. isolates
with NCBI genomes and plant pathogenic species

| | | | ANI vs closest | |
|---|---|---|---|---|
| | ANI vs. | Closest related NCBI | related NCBI | Novel strain or |
| Isolate Name | CFBP3419 | genome | genome | species? |
| P3-Gcland-NS-Dand-IS-96-1 | 0.86 | C. sp. MMLR14_010 | 0.93 | Novel species |
| | | MMLR14_010 | | (Cluster C) |
| P3-Gcland-NS-Dand-IS-26-1 | 0.86 | C. sp. MMLR14_010 | 0.93 | Novel species |
| | | MMLR14_010 | | (Cluster C) |
| P2-Gtab-NS-Kalkallo-IS-65-1 | 0.86 | C. sp. ER1/6 ER1/6 | 0.90 | Novel species |
| | | | | (Cluster A) |
| P2-Gtab-NS-Kalkallo-IS-64-1 | 0.87 | C. sp. MCBA15_008 | 0.97 | Novel strain |
| | | MCBA15_008 | | (Cluster B) |
| P3-Gtab-NS-Kalkallo-IS-74-1 | 0.86 | C. sp. ER1/6 ER1/6 | 0.90 | Novel species |
| | | | | (Cluster A) |
| P3-Gcland-NS-Dand-IS-27-1 | 0.86 | C. sp. MMLR14_010 | 0.93 | Novel species |
| | | MMLR14_010 | | (Cluster C) |

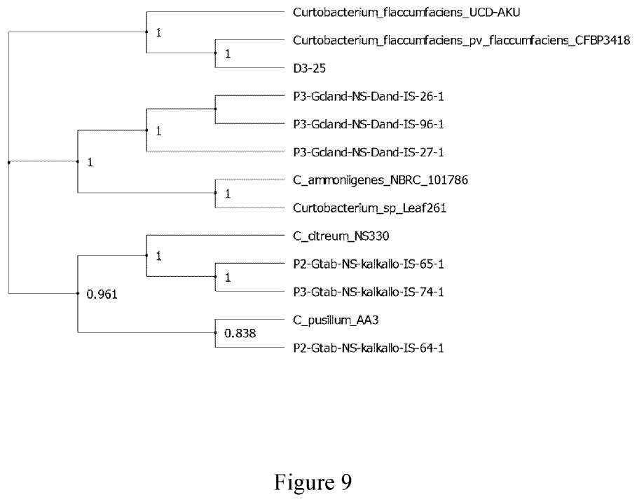

Six Curtobacterium sp. genome sequences that were publicly available on NCBI and a previously patented Curtobacterium flaccumfaciens strain D3-25 were acquired and used for pan-genome/comparative genome sequence analysis alongside the six Curtobacterium isolates from the Glycine microbiome (P3-Gcland-NS-Dand-IS-96-1, P3-Gcland-NS-Dand-IS-26-1, P2-Gtab-NS-kalkallo-IS-65-1, P2-Gtab-NS-kalkallo-IS-64-1, P3-Gtab-NS-kalkallo-IS-74-1, and P3-Gcland-NS-Dand-IS-27-1). A total of 36 genes that were shared by all Curtobacterium sp. strains were identified by running Roary (Page et al. 2015). PRANK (Löytynoja 2014) was then used to perform a codon aware alignment. A maximum-likelihood phylogenetic tree was inferred using FastTree (Price et al., 2010) with Jukes-Cantor Joins distances and Generalized Time-Reversible and CAT approximation model. Local support values for branches were calculated using 1000 resamples with the Shimodaira-Hasegawa test. These results are shown in FIG. 9.

### Example 4—Genome Sequence Features Supporting the Biofertilizer Niche of the Bacterial Strains

The presence of PGP genes in the annotated genomes of P3-Gcland-NS-Dand-IS-96-1, P3-Gcland-NS-Dand-IS-26-1, P2-Gtab-NS-kalkallo-IS-65-1, P2-Gtab-NS-kalkallo-IS-64-1, P3-Gtab-NS-kalkallo-IS-74-1, and P3-Gcland-NS-Dand-IS-27-1 were assessed. Previously reported PGP genes from Curtobacterium sp. strains were targeted, including genes encoding phosphate solubilisation and assimilation (17 genes), as well as indole-3-acetic acid production and transportation (3 genes) (Bulgari et al. 2014, Vimal et al, 2019).

As shown in Table 5, the genomes of P3-Gcland-NS-Dand-IS-96-1, P3-Gcland-NS-Dand-IS-26-1, P2-Gtab-NS-kalkallo-IS-65-1, P2-Gtab-NS-kalkallo-IS-64-1, P3-Gtab-NS-kalkallo-IS-74-1, P1-Gtab-NS-kalkallo-IS-74-1, and P3-Gcland-NS-Dand-IS-27-1 isolates were found to possess a number of PGP genes. In particular, 16 genes associated with phosphate solubilisation and assimilation were identified, including the glucose-1-dehydrogenase (gcd) gene for inorganic phosphate solubilisation (de Werra et al. 2009) and the phn clusters of 9 genes for organic phosphate (phosphonates) solubilisation (Lugtenberg & Kamilova 2009). Phosphate solubilisation can occur via phosphorylation of glucose to glucose-6-phosphate or through the direct oxidation of glucose to gluconate followed by induction of the Entner-Doudoroff pathway (Sashidhar et al, 2010). The presence of UGD (K00012) in nine out of twelve isolates potentially indicates the ability to solubilise inorganic phosphate. The presence of GDH (K00034) in six out of twelve isolates potentially indicates the ability to solubilise inorganic phosphate. However, the gluconic acid dehydrogenase (gad) gene for inorganic phosphate solubilisation (de Werra et al. 2009) was not found in any of the isolates. Sequence comparison of the PGP genes between bacterial strains P3-Gcland-NS-Dand-IS-96-1, P3-Gcland-NS-Dand-IS-26-1, P2-Gtab-NS-kalkallo-IS-65-1, P2-Gtab-NS-kalkallo-IS-64-1, P1-Gtab-NS-kalkallo-IS-74-1, and P3-Gcland-NS-Dand-IS-27-1 showed a broader complement of genes when compared to C. flaccumfaciens strain CFBP3419 (see Table 5).

TABLE 5
Presence of isolate PGP genes compared to
Curtobacterium flaccumfaciens strain CFBP3419

| | | | | | | | |
|---|---|---|---|---|---|---|---|
| PGP gene | 96 | 26 | 65 | 64 | 74 | 27 | CFBP3419 |

Phosphate solubilisation

| | | | | | | | |
|---|---|---|---|---|---|---|---|
| ugd | X | X | X | X | X | X | X |
| gcd | X | X | — | — | X | — | |
| gad | — | — | — | — | — | — | |

Phosphonate cluster (phn)

| | | | | | | | |
|---|---|---|---|---|---|---|---|
| phnA | — | — | — | — | — | — | — |
| phnB | X | X | — | — | — | — | — |
| phnC | X | X | — | X | — | X | X |
| phnD | X | X | — | X | — | X | X |
| phnE | X | X | — | X | — | X | X |
| phnW | — | X | X | — | — | — | — |
| phnX | — | X | X | | — | — | — |
| ppd | X | X | X | | — | — | — |
| pepM | X | x | X | | — | — | — |

Phosphate transporter (pst)

| | | | | | | | |
|---|---|---|---|---|---|---|---|
| pstS | X | X | X | X | X | X | X |
| pstA | X | X | X | X | X | X | X |
| pstB | X | X | X | X | X | X | X |
| pstC | X | X | X | X | X | X | X |
| phoP | — | — | X | X | X | X | — |
| phoR | — | — | X | X | X | — | X |

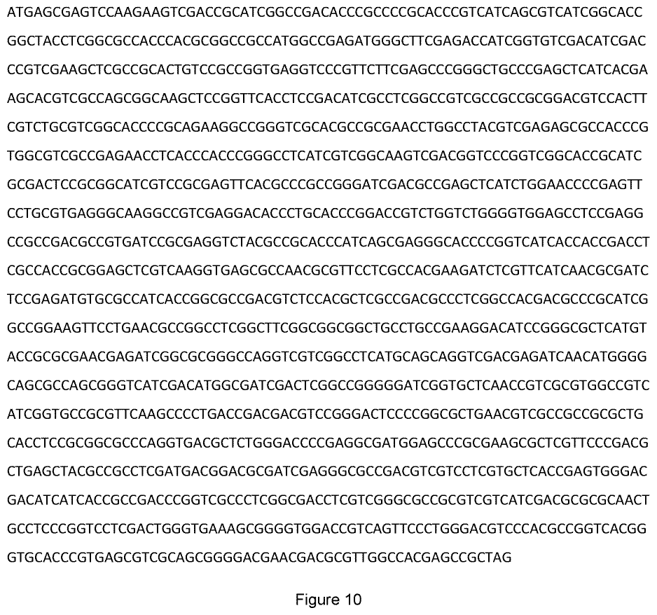

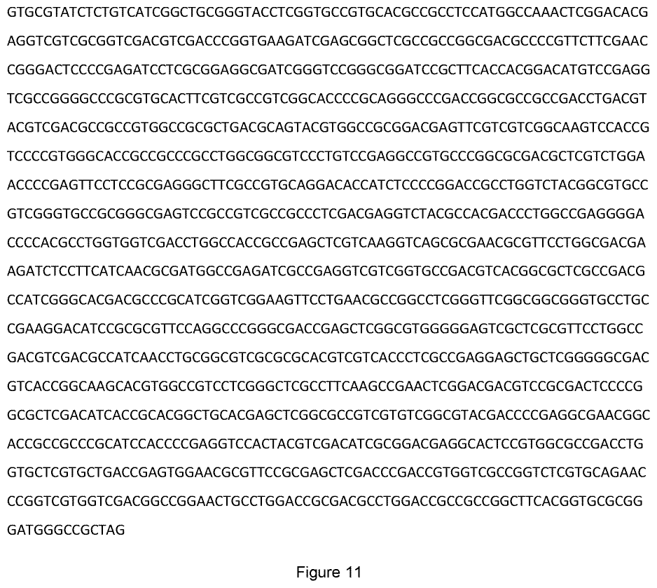

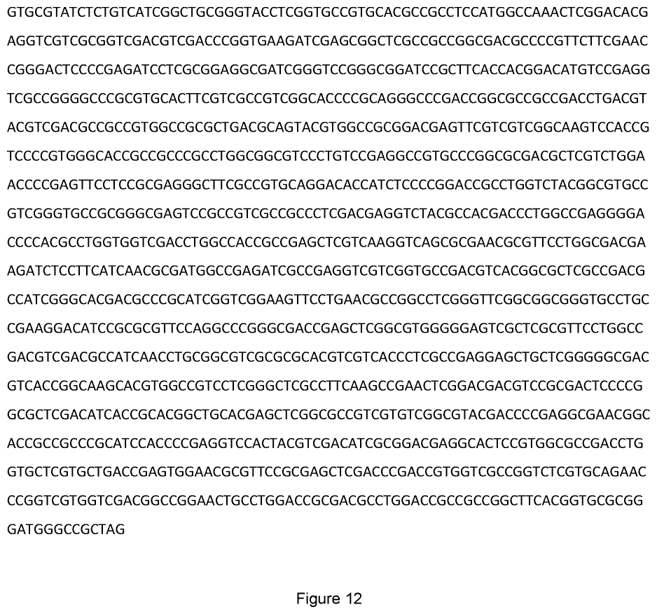

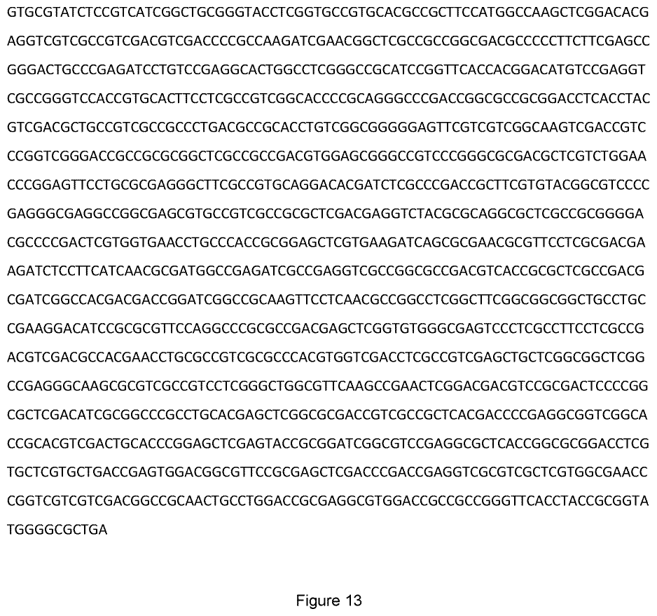

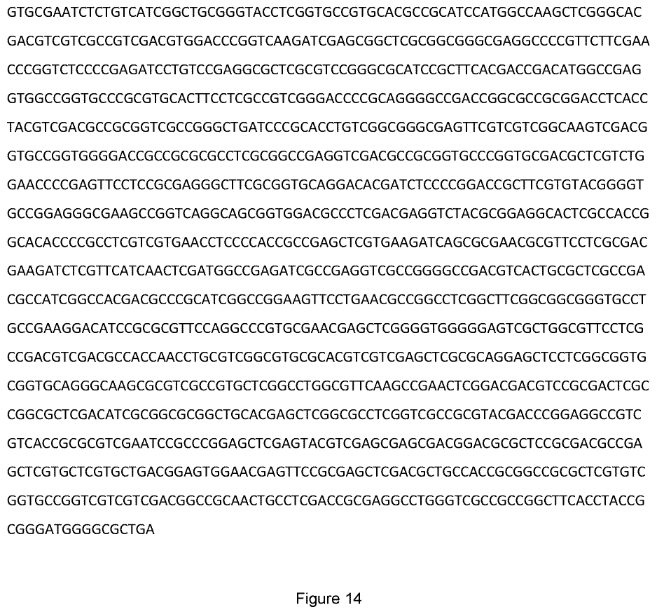

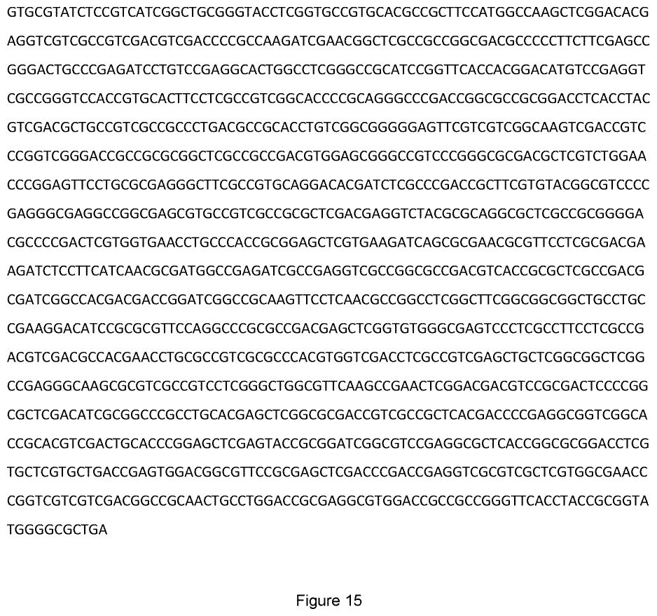

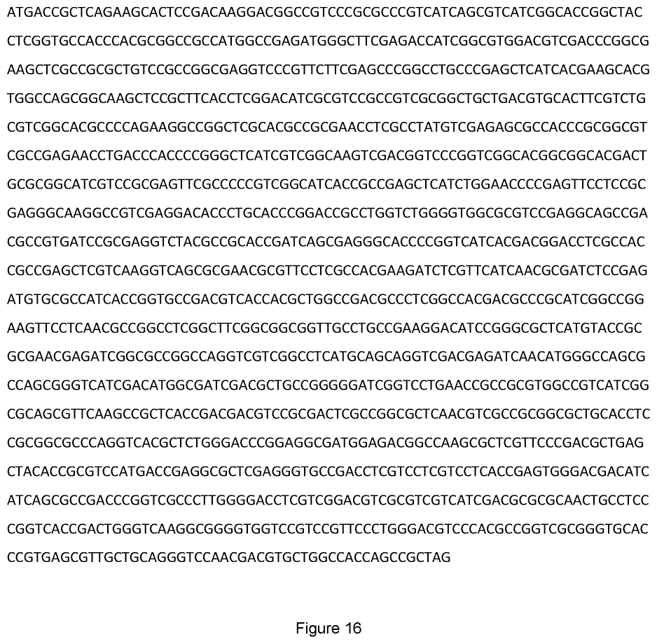

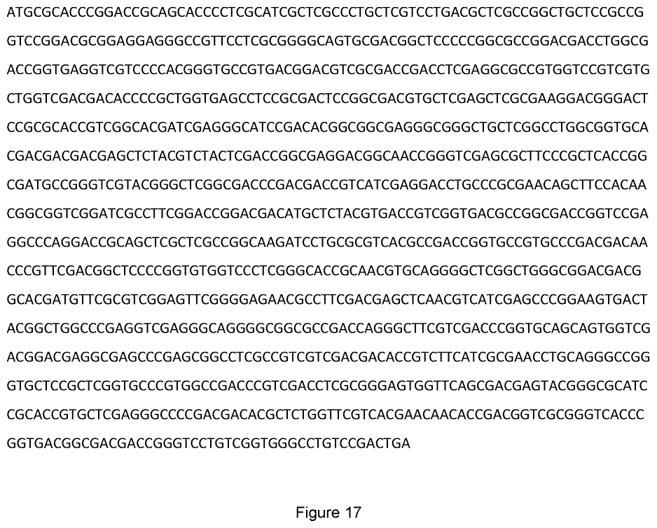

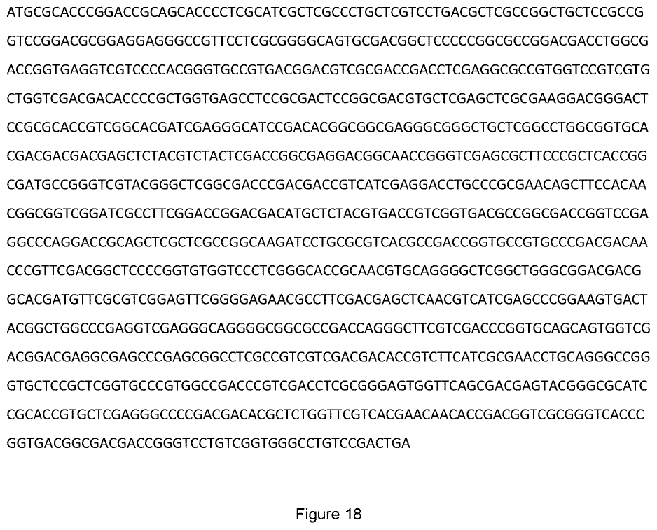

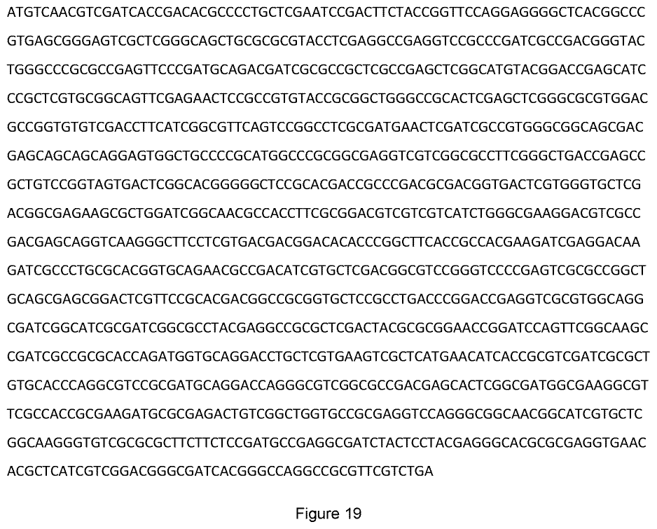

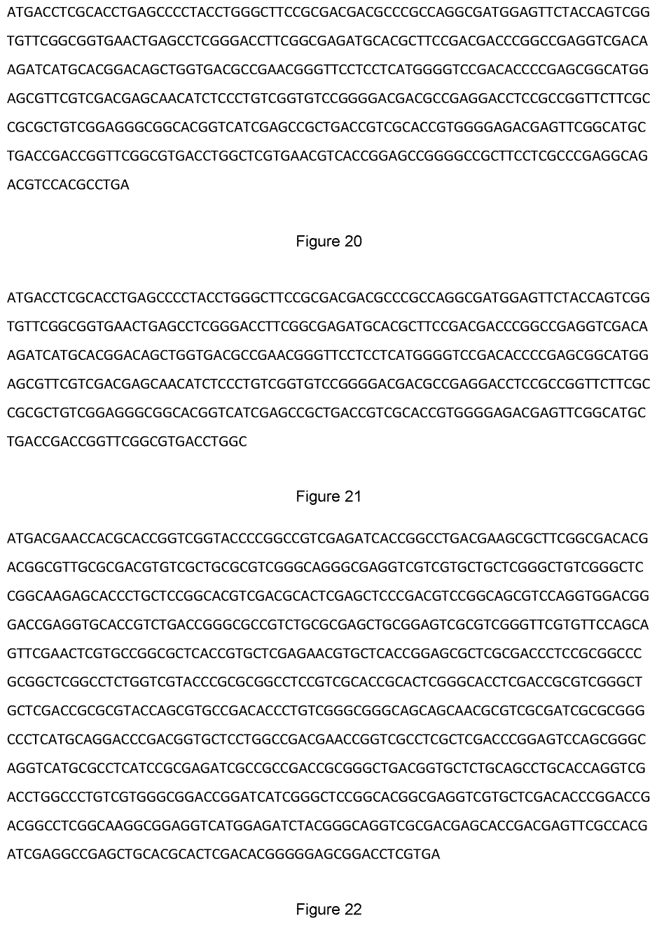

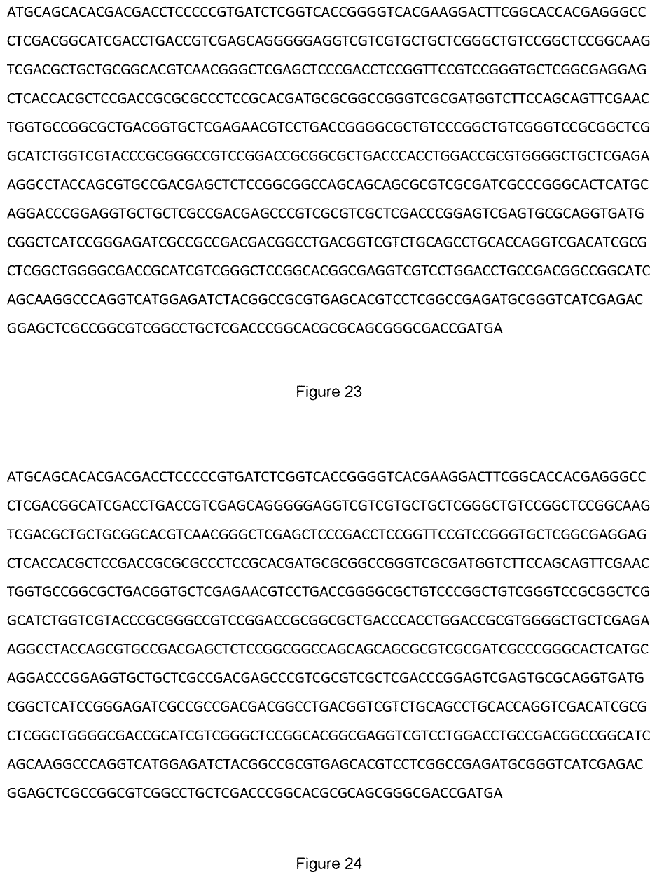

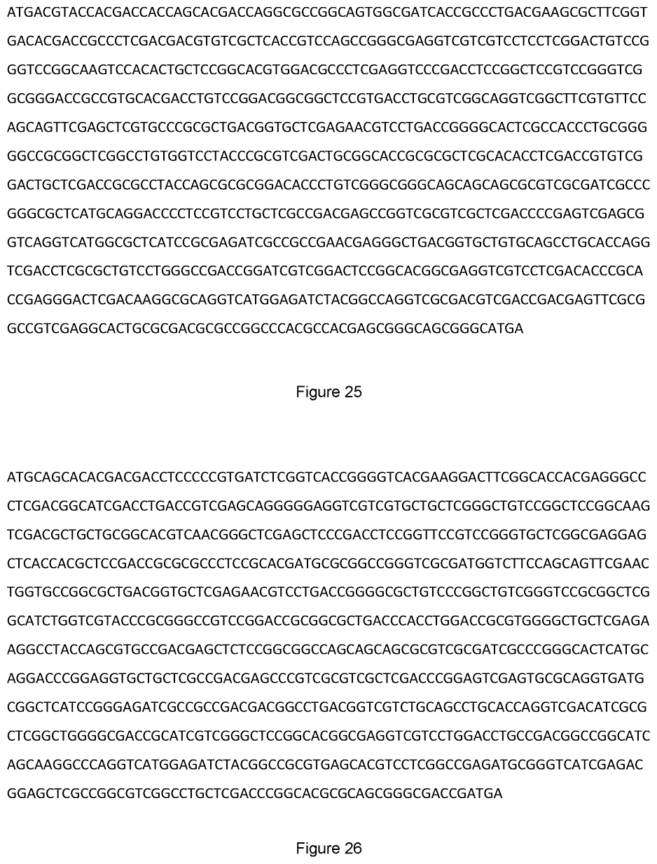

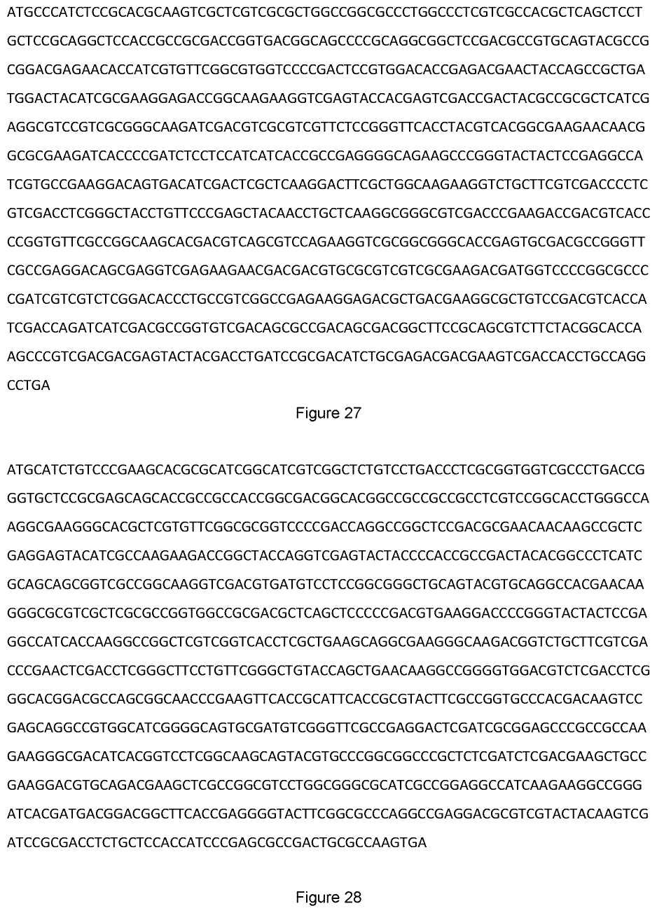

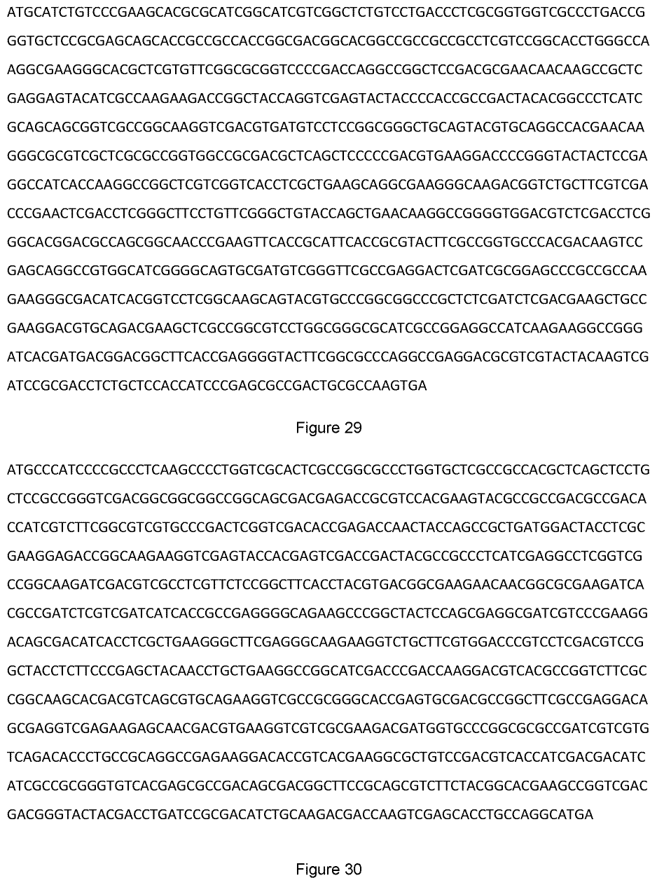

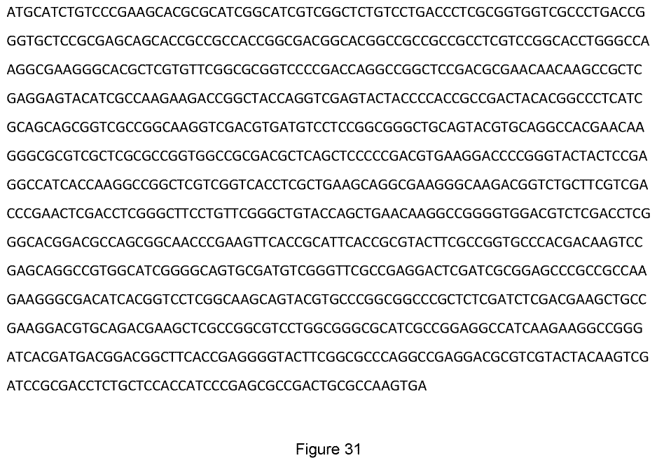

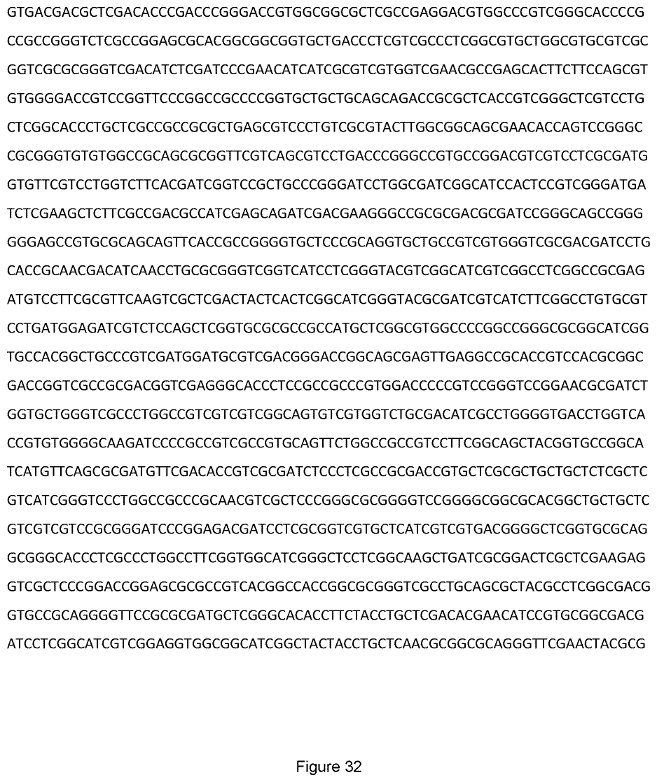

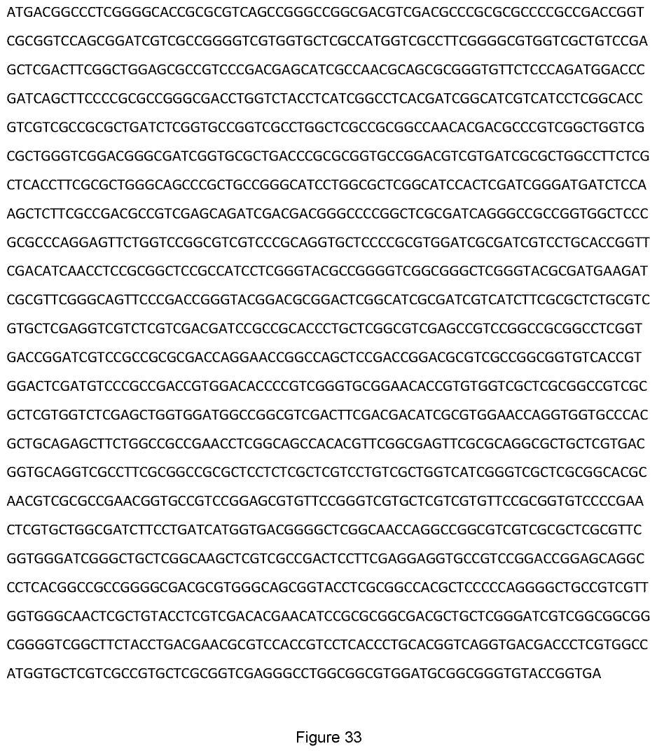

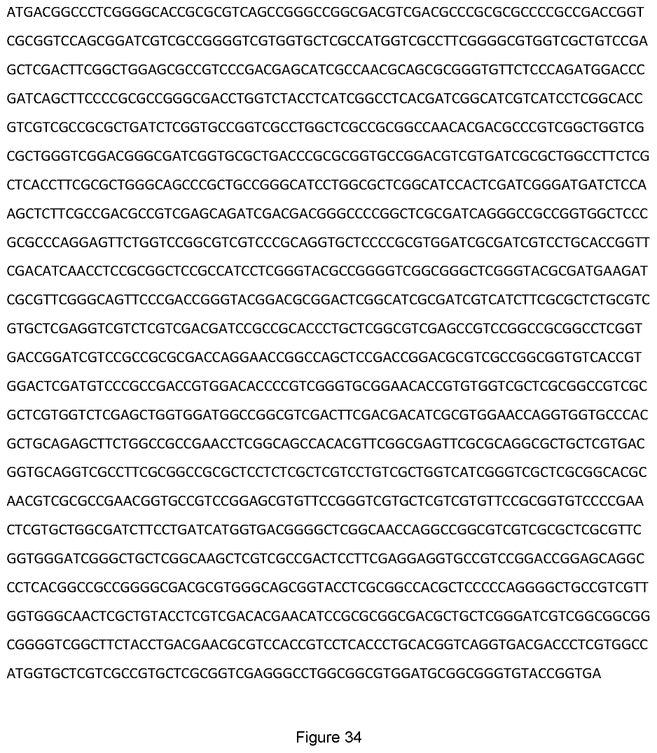

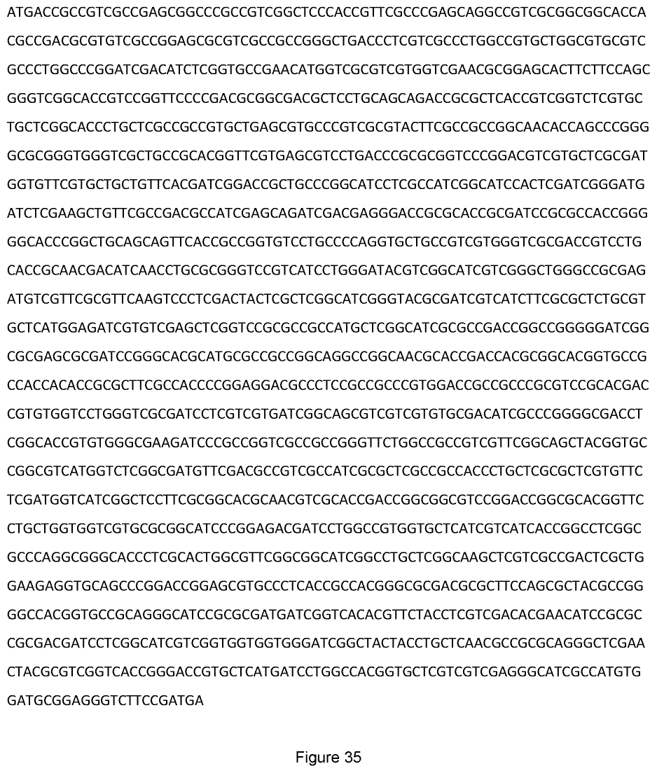

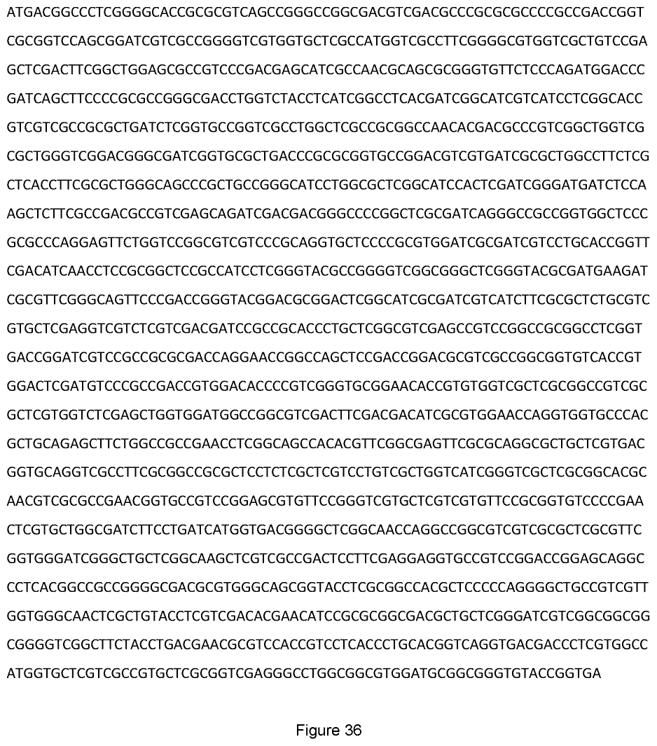

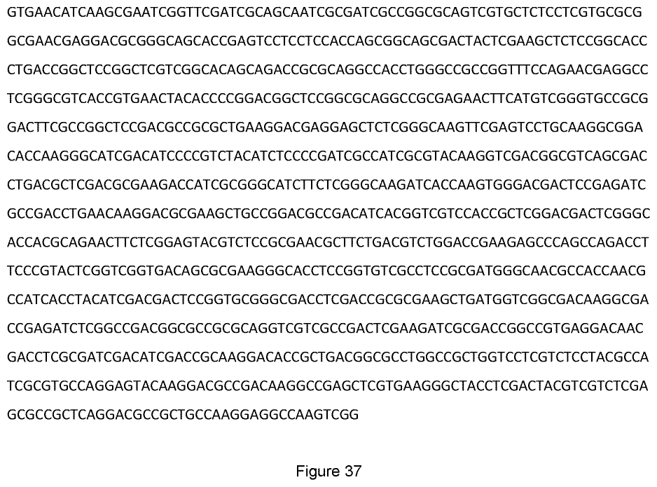

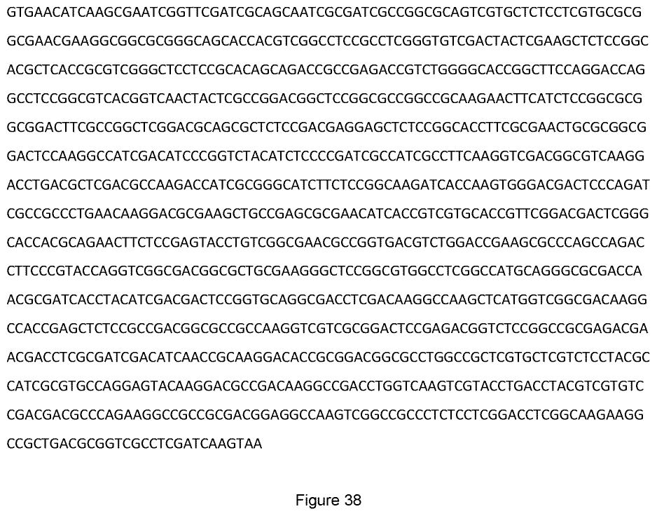

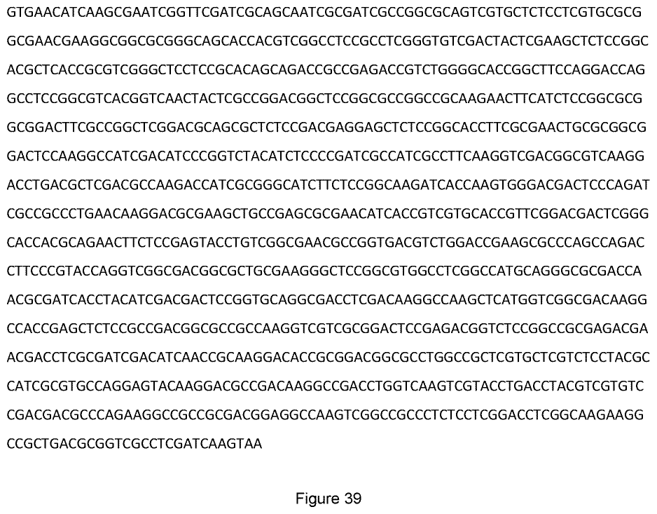

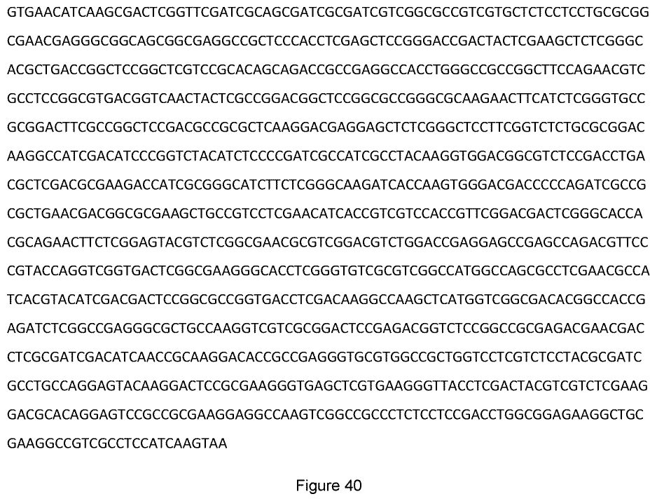

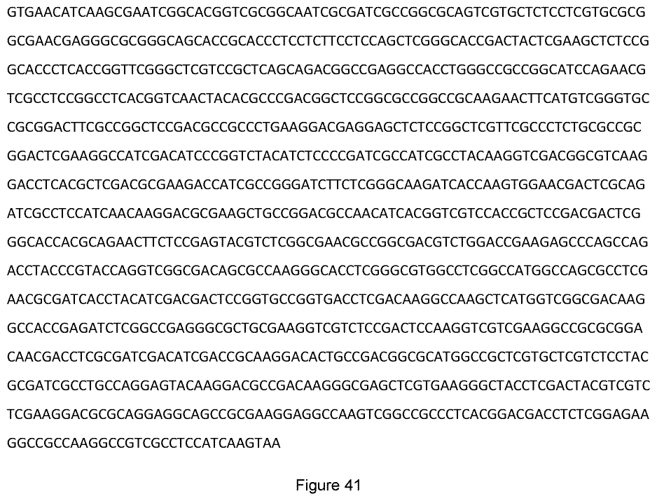

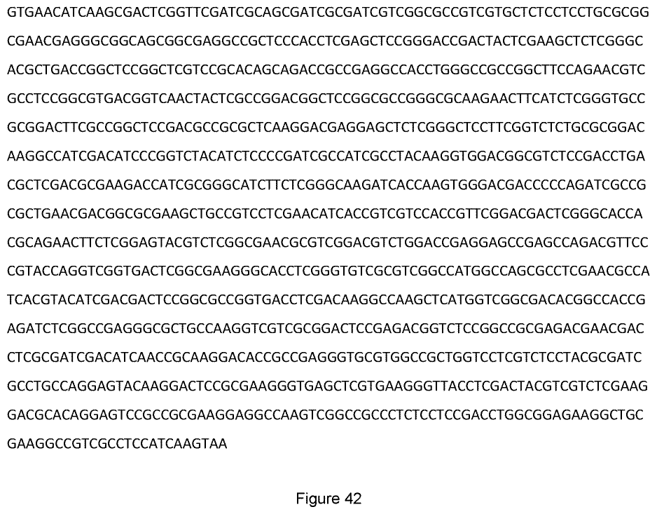

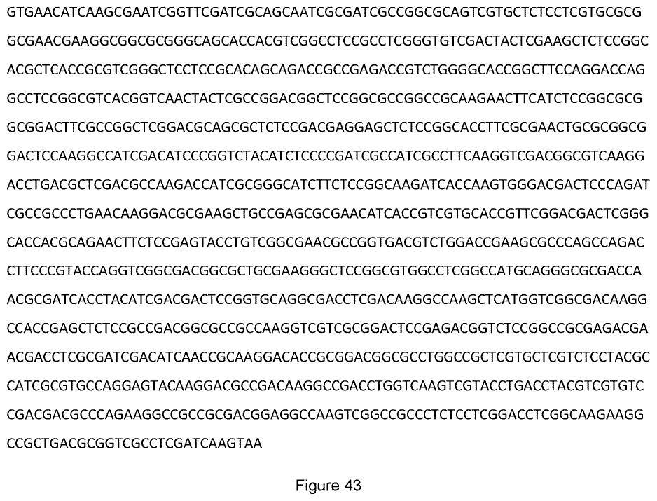

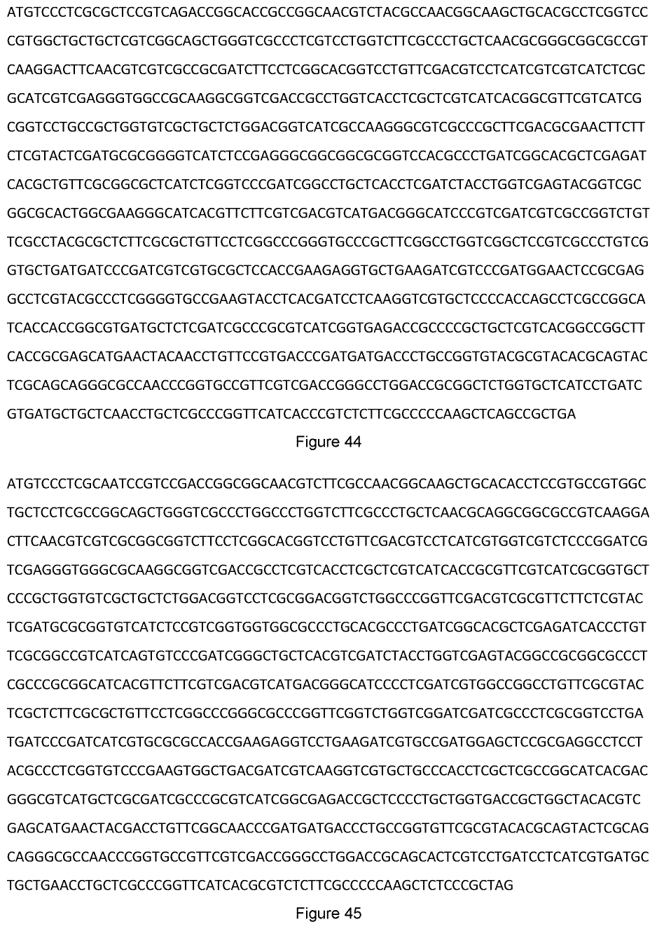

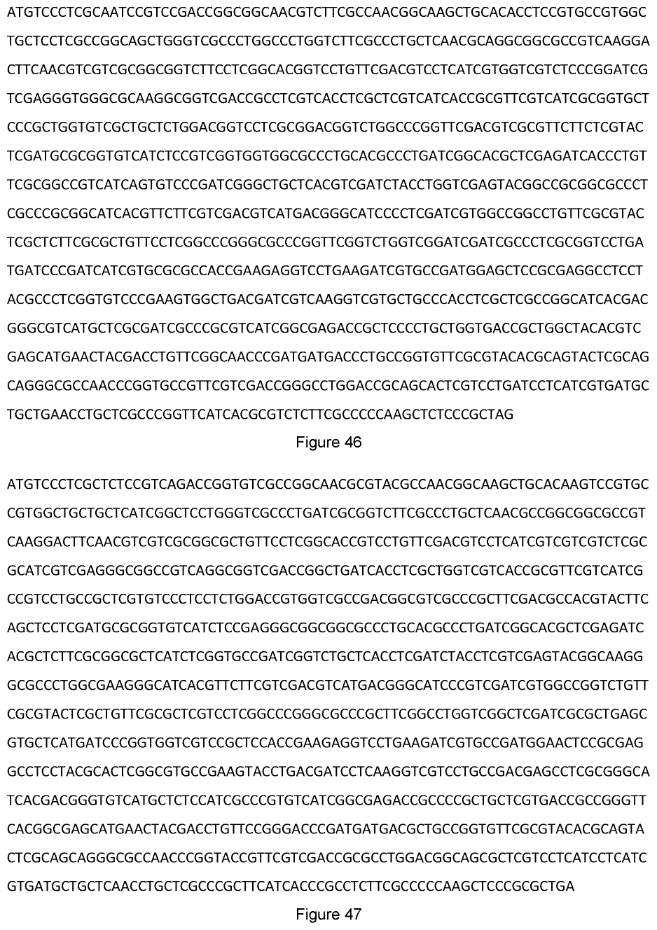

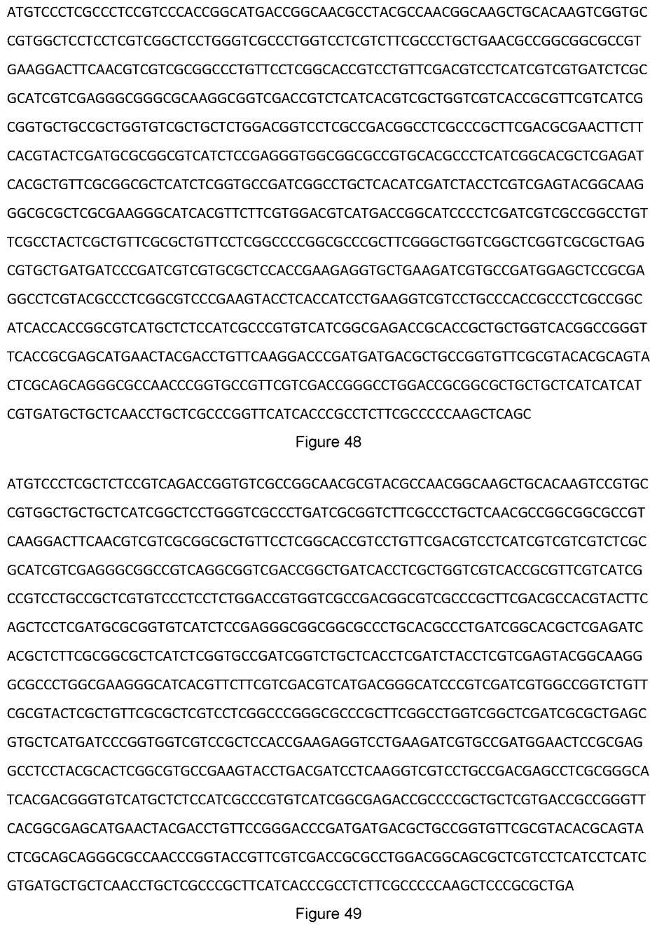

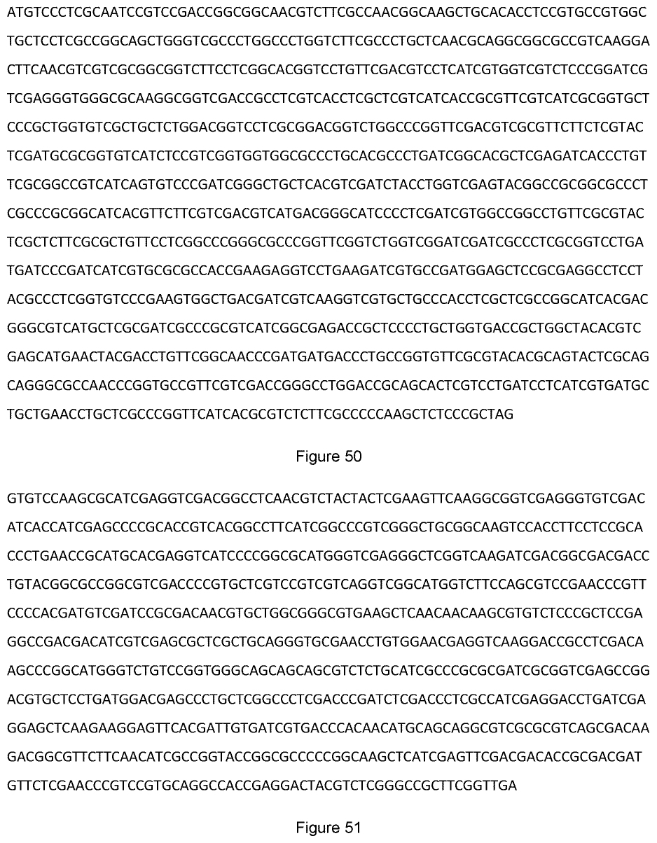

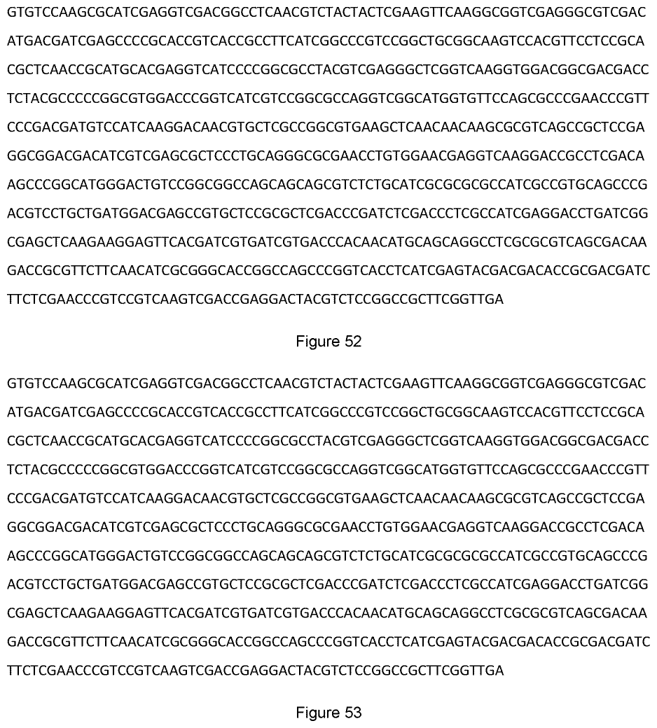

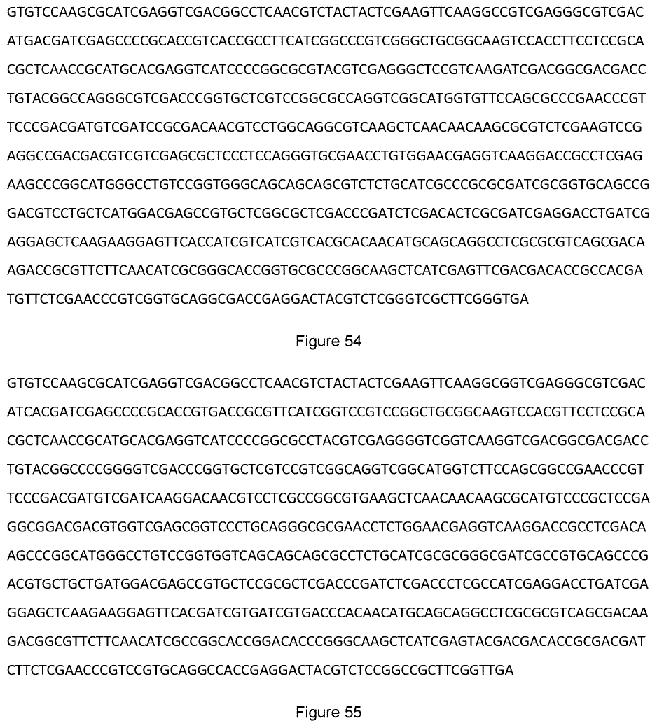

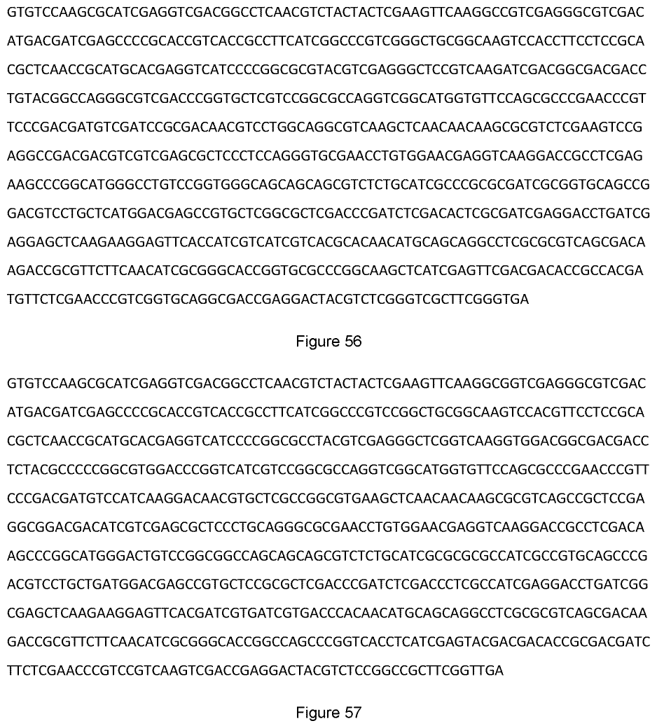

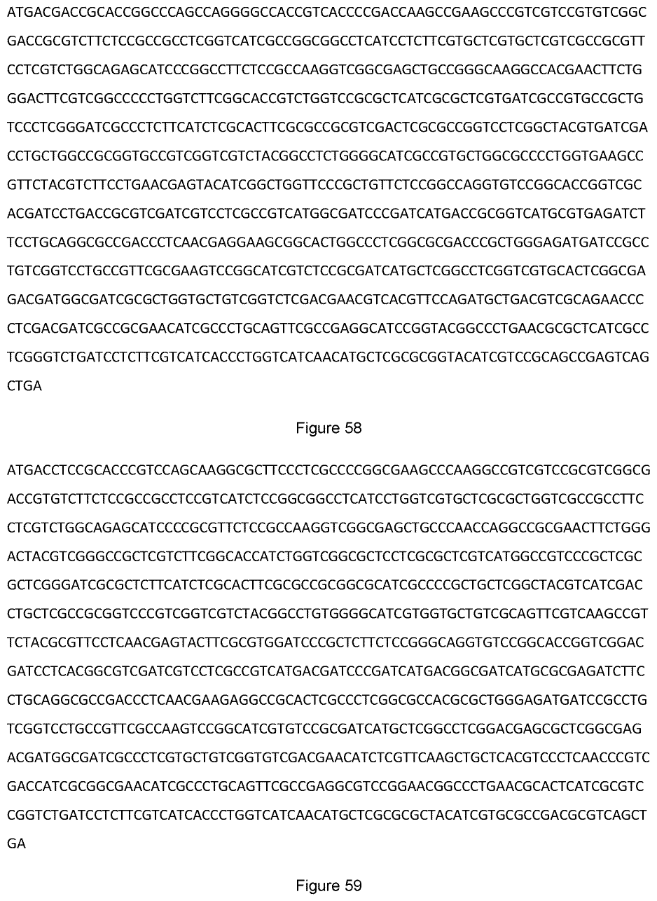

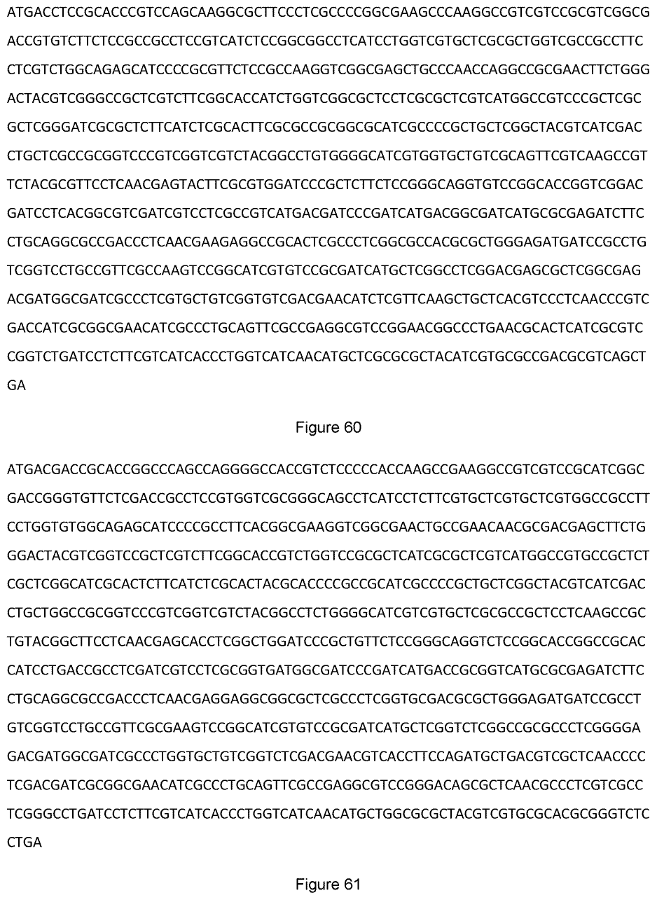

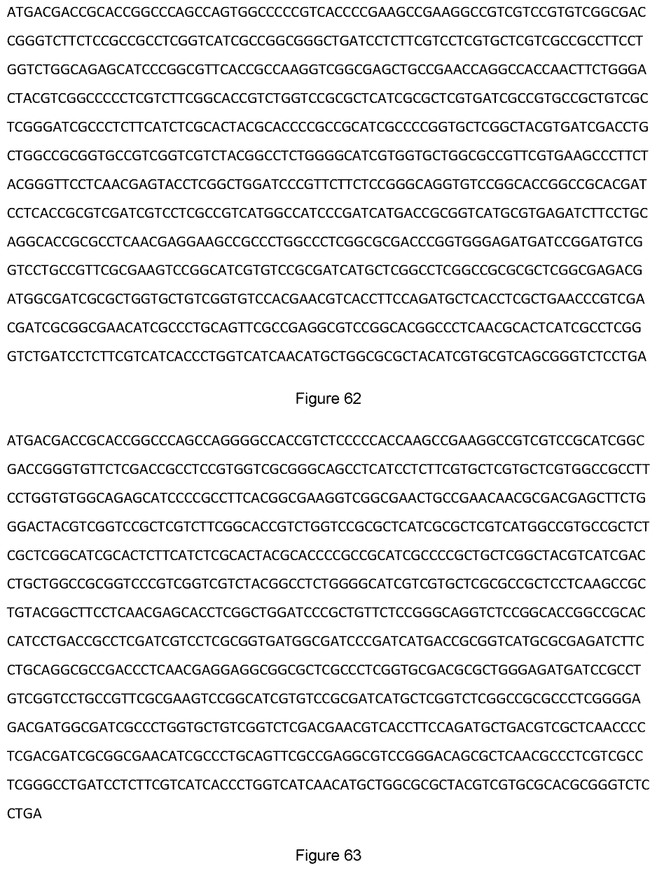

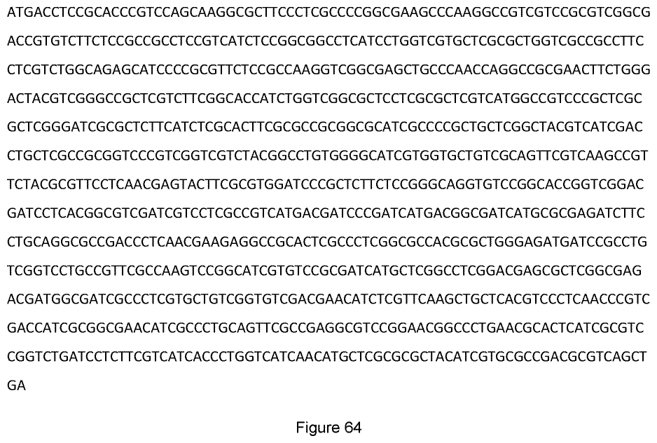

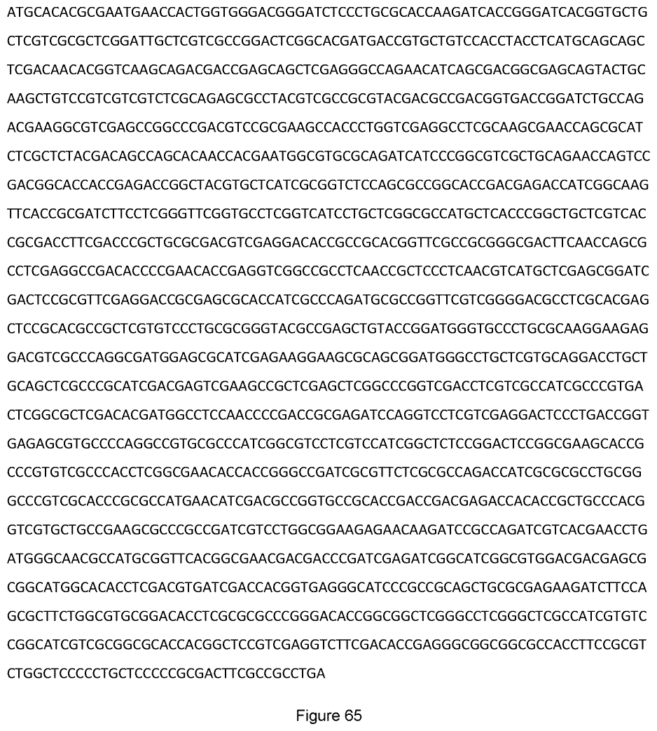

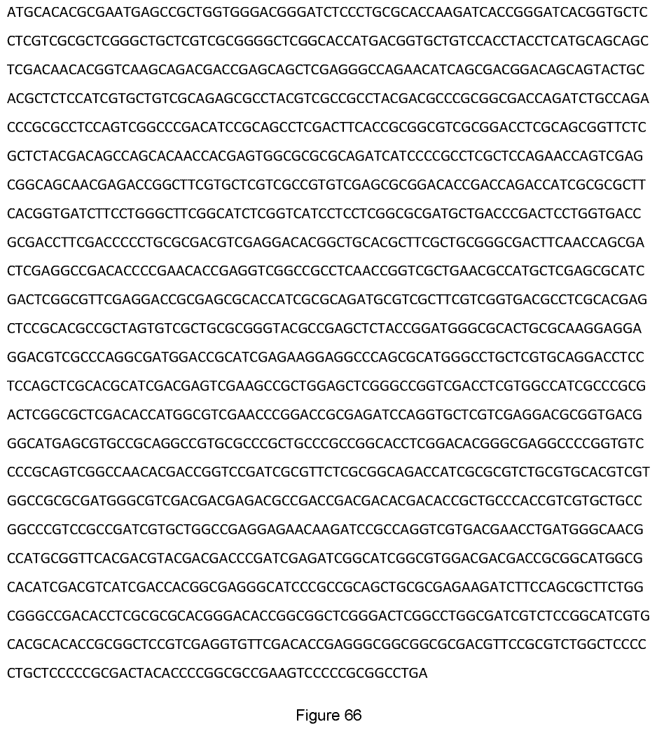

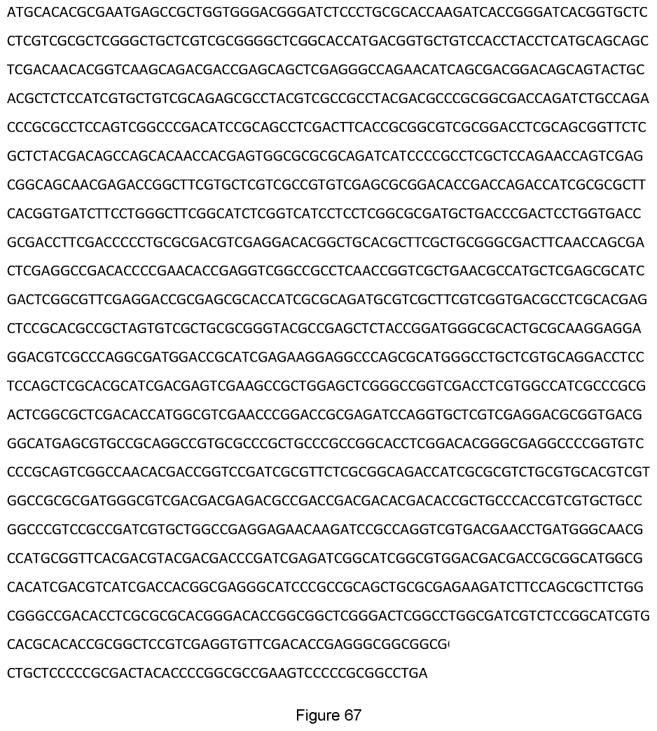

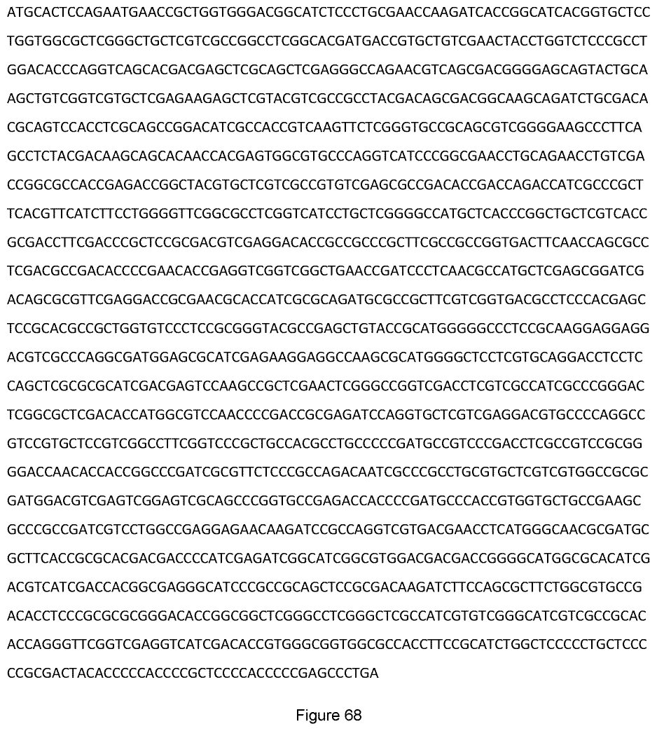

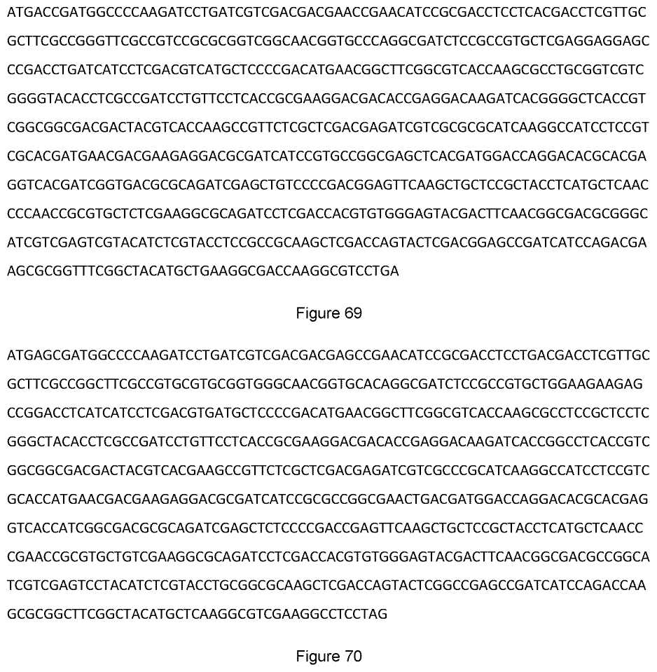

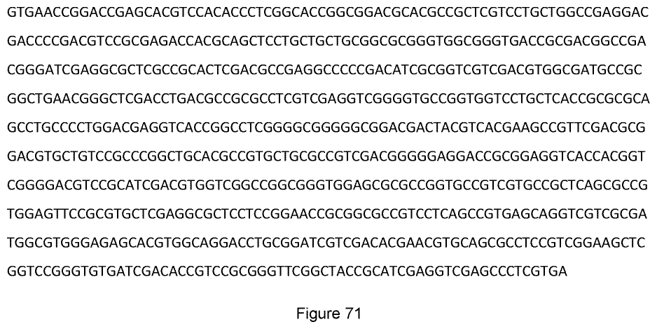

The PGP gene sequences identified in the annotated genomes of Curtobacterium flaccumfaciens strain CFBP3419 and the Curtobacterium spp. described herein are shown in FIGS. 10 to 71 and correspond to SEQ ID NOs: 7 to 68. The specific PGP genes and associated SEQ ID NOs are shown in Table 6.

TABLE 6
Sequence listing of PGP genes identified in
Curtobacterium flaccumfaciens strain CFBP3419 and in
the new Curtobacterium spp. plant species

| | | SEQ ID |
|---|---|---|
| PGP gene | Strain ID | NO |
| ugd | CFBP3419 | 7 |
| | P3-Gcland-NS-Dand-IS-96-1 | 8 |
| | P3-Gcland-NS-Dand-IS-26-1 | 9 |
| | P2-Gtab-NS-Kalkallo-IS-65-1 | 10 |
| | P2-Gtab-NS-Kalkallo-IS-64-1 | 11 |
| | P3-Gtab-NS-Kalkallo-IS-74-1 | 12 |
| | P3-Gcland-NS-Dand-IS-27-1 | 13 |
| gcd | P3-Gcland-NS-Dand-IS-96-1 | 14 |
| | P3-Gcland-NS-Dand-IS-26-1 | 15 |
| | P3-Gcland-NS-Dand-IS-74-1 | 16 |
| phnB | P3-Gcland-NS-Dand-IS-96-1 | 17 |
| | P3-Gcland-NS-Dand-IS-26-1 | 18 |
| phnC | CFBP3419 | 19 |
| | P3-Gcland-NS-Dand-IS-96-1 | 29 |
| | P3-Gcland-NS-Dand-IS-26-1 | 21 |
| | P2-Gtab-NS-Kalkallo-IS-64-1 | 22 |
| | P3-Gcland-NS-Dand-IS-27-1 | 23 |
| phnD | CFBP3419 | 24 |
| | P3-Gcland-NS-Dand-IS-96-1 | 25 |
| | P3-Gcland-NS-Dand-IS-26-1 | 26 |
| | P2-Gtab-NS-Kalkallo-IS-64-1 | 27 |
| | P3-Gcland-NS-Dand-IS-27-1 | 28 |
| phnE | CFBP3419 | 29 |
| | P3-Gcland-NS-Dand-IS-96-1 | 30 |
| | P3-Gcland-NS-Dand-IS-26-1 | 31 |
| | P2-Gtab-NS-Kalkallo-IS-64-1 | 32 |
| | P3-Gcland-NS-Dand-IS-27-1 | 33 |
| pstS | CFBP3419 | 34 |
| | P3-Gcland-NS-Dand-IS-96-1 | 35 |
| | P3-Gcland-NS-Dand-IS-26-1 | 36 |
| | P2-Gtab-NS-Kalkallo-IS-65-1 | 37 |
| | P2-Gtab-NS-Kalkallo-IS-64-1 | 38 |
| | P3-Gtab-NS-Kalkallo-IS-74-1 | 39 |
| | P3-Gcland-NS-Dand-IS-27-1 | 40 |
| pstA | CFBP3419 | 41 |
| | P3-Gcland-NS-Dand-IS-96-1 | 42 |
| | P3-Gcland-NS-Dand-IS-26-1 | 43 |
| | P2-Gtab-NS-Kalkallo-IS-65-1 | 44 |
| | P2-Gtab-NS-Kalkallo-IS-64-1 | 45 |
| | P3-Gtab-NS-Kalkallo-IS-74-1 | 46 |
| | P3-Gcland-NS-Dand-IS-27-1 | 47 |
| pstB | CFBP3419 | 48 |
| | P3-Gcland-NS-Dand-IS-96-1 | 49 |
| | P3-Gcland-NS-Dand-IS-26-1 | 50 |
| | P2-Gtab-NS-Kalkallo-IS-65-1 | 51 |
| | P2-Gtab-NS-Kalkallo-IS-64-1 | 52 |
| | P3-Gtab-NS-Kalkallo-IS-74-1 | 53 |
| | P3-Gcland-NS-Dand-IS-27-1 | 54 |
| pstC | CFBP3419 | 55 |
| | P3-Gcland-NS-Dand-IS-96-1 | 56 |
| | P3-Gcland-NS-Dand-IS-26-1 | 57 |
| | P2-Gtab-NS-Kalkallo-IS-65-1 | 58 |
| | P2-Gtab-NS-Kalkallo-IS-64-1 | 59 |
| | P3-Gtab-NS-Kalkallo-IS-74-1 | 60 |
| | P3-Gcland-NS-Dand-IS-27-1 | 61 |
| phoR | CFBP3419 | 62 |
| | P2-Gtab-NS-Kalkallo-IS-65-1 | 63 |
| | P3-Gtab-NS-Kalkallo-IS-74-1 | 64 |
| | P3-Gcland-NS-Dand-IS-27-1 | 65 |
| phoP | P2-Gtab-NS-Kalkallo-IS-65-1 | 66 |
| | P2-Gtab-NS-Kalkallo-IS-64-1 | 67 |
| | P3-Gtab-NS-Kalkallo-IS-74-1 | 68 |

### Example 5—In Vitro Assays Supporting the Endophytic Niche and Beneficial Traits of the Novel Bacterial Strains

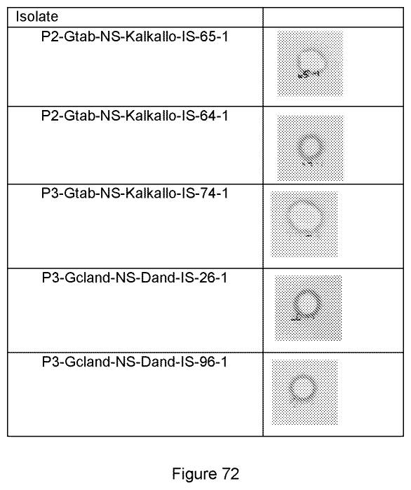

An in vitro assay was developed to assess the ability of Curtobacterium sp. P3-Gcland-NS-Dand-IS-96-1, P3-Gcland-NS-Dand-IS-26-1, P2-Gtab-NS-kalkallo-IS-65-1, and P2-Gtab-NS-kalkallo-IS-64-1 to solubilise phosphate. The isolates were grown on Pikovskayas agar to confirm the ability to solubilise phosphate in vitro. Pikovskayas agar is designed to detection of phosphate-solubilising bacteria from soil (Paul & Sundara 1971). Yeast extract in the medium provides nitrogen and other nutrients necessary to support bacterial growth, while dextrose acts as an energy source. As phosphate-solubilising bacteria grow on Pikovskayas agar, a clear zone forms around the colony as phosphate is solubilised from the surrounding media. The bacterial strains were removed from −80° C. glycerol stock and streaked on R2A plates and grown at 21.5° C. for 48 hours. The strains were grown on Pikovskayas agar and incubated at 21.5° C. for 7 days. After 7 days of growth, the agar plates were photographed and examined for signs of clearing. As shown in FIG. 72, each isolate showed a clear zone under the colony when grown on Pikovskayas agar, confirming the ability to solubilise phosphate.

### Example 6—In Planta Inoculations in Wheat and Soybean Supporting Endophytic Niche and Beneficial Traits of Curtobacterium Strains

### Phosphate Solubilisation

To assess the phosphate solubilisation effect of the isolate in soybean, a seedling assay was established. Curtobacterium sp. P3-Gcland-NS-Dand-IS-96-1 and P3-Gcland-NS-Dand-IS-26-1, P2-Gtab-NS-kalkallo-IS-64-1 were cultured in Lysogeny Broth (LB) overnight at 26° C. The following day soybean seeds were sterilised by soaking in 80% ethanol for 3 mins, left to dry out in the laminar flow for 15 minutes, and then washed 5 times in sterile distilled water. An OD reading was taken to determine the CFU/mL. Seeds were soaked in the inoculated LB broth for 4 hours at 26° C. in a shaking incubator. As a control, seeds were soaked in LB without bacteria for 4 hours at 26° C. in a shaking incubator. Six inoculated seeds were then placed on moist sterile filter paper in sterile petri plates and allowed to grow for three days.

There were five replicates per treatment. Single Glycine max seeds that germinated on the same day were placed on Pikovskaya agar and allowed to grow for seven days. After a total ten days of growth, the plates were photographed and the root and shoot lengths seedlings were measured. Data was statistically analysed using a one-way ANOVA and Tukey test to detect the presence of any significant difference (p≤0.05) between treatments using OriginPro 2018 (Version b9.5.1.195).

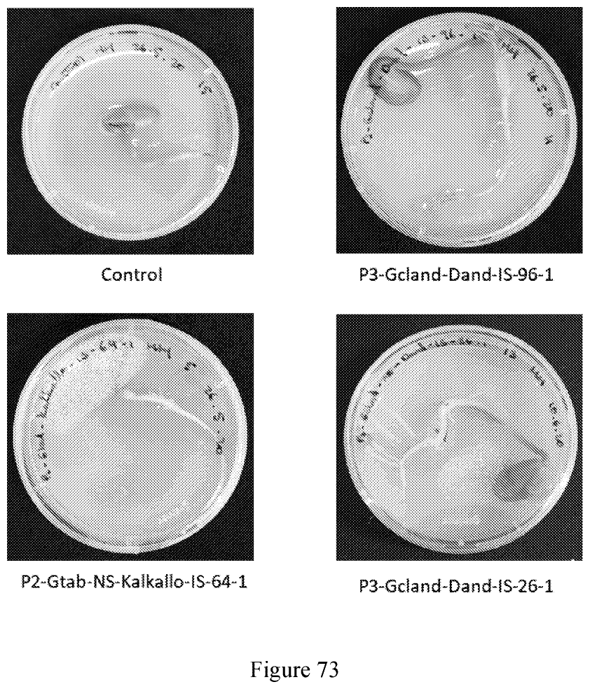

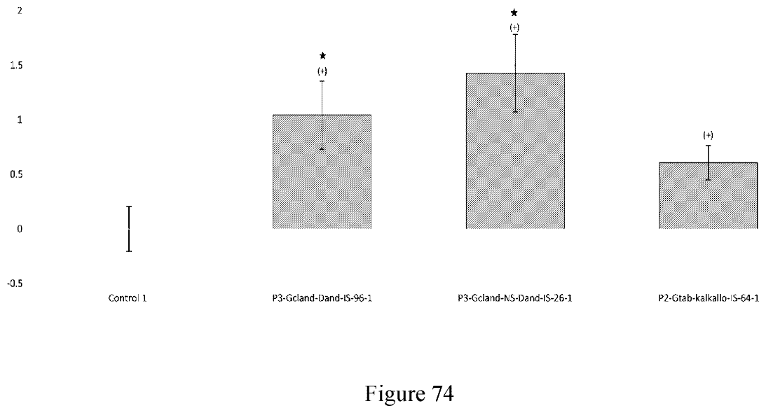

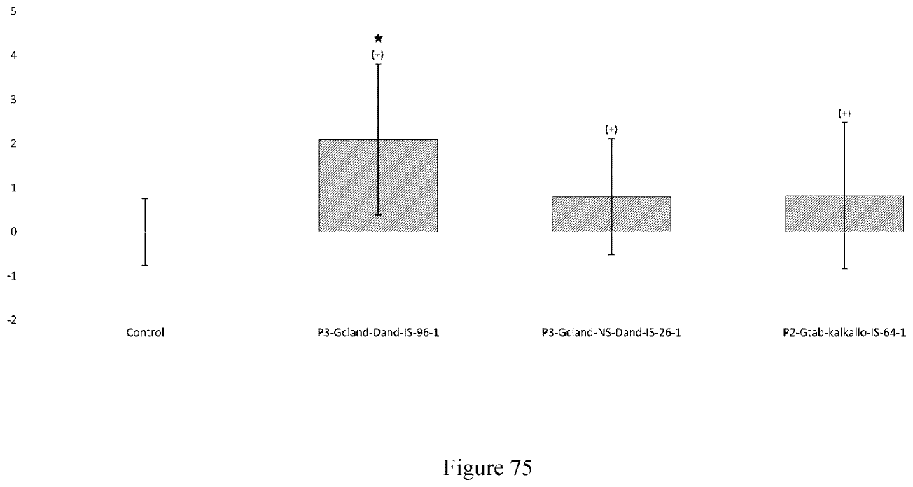

As shown in FIGS. 73 and 74, Glycine max seedlings treated with P3-Gcland-Dand-IS-96-1 and P3-Gcland-NS-Dand-IS-26-1 had significantly longer shoots than the control. As shown in FIGS. 73 and 75, Glycine max seedlings treated with P3-Gcland-Dand-IS-96-1 had significantly longer roots than the control. Glycine max seedlings treated with P3-Gcland-Dand-IS-64-1 had longer roots than the control, however the difference was not significant. Both isolates had shown the potential to solubilise phosphate in in silico and in vitro assays.

### Plate Growth Assay

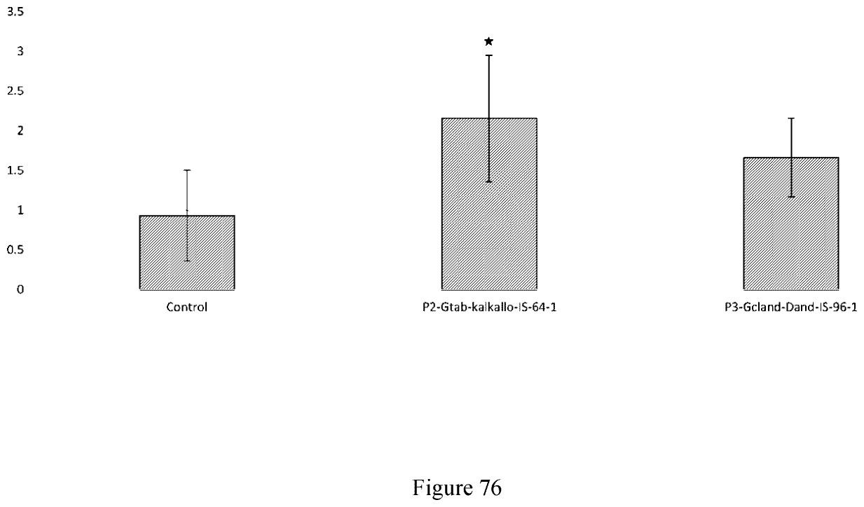

To assess the growth effect of isolates (P3-Gcland-NS-Dand-IS-96-1 and P2-Gtab-NS-kalkallo-IS-64-1) in soybean, a seedling assay was established. As P3-Gcland-Dand-IS-96-1 and P3-Gcland-NS-Dand-IS-26-1 are so closely related, P3-Gcland-Dand-IS-96-1 was chosen as a representative. The soybean seeds were sterilised and then inoculated with the novel endophytes using the method described above. As a control, seeds were soaked in LB without bacteria for 4 hours at 26° C. in a shaking incubator. Six inoculated seeds were then placed on moist sterile filter paper in sterile petri plates. There were five replicates per treatment. After ten days of growth, the root and shoot lengths of Glycine max seedlings that had germinated on the same day were measured. Data was statistically analysed using a one-way ANOVA and Tukey test to detect the presence of any significant difference (p s 0.05) between treatments using OriginPro 2018 (Version b9.5.1.195). Glycine seedlings inoculated with Curtobacterium sp P3-Gcland-NS-Dand-IS-96-1 had a significant increase in root length, with a percentage increase of 131% (see FIG. 76). Glycine seedlings inoculated with Curtobacterium sp P2-Gtab-NS-kalkallo-IS-64-1 showed an increase in root length of 78%, however this difference was not statistically significant.

### Pot Growth Assay

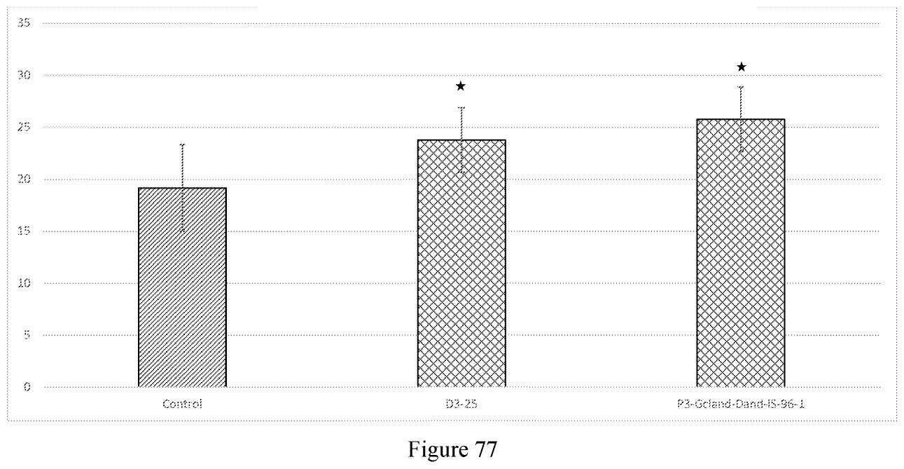

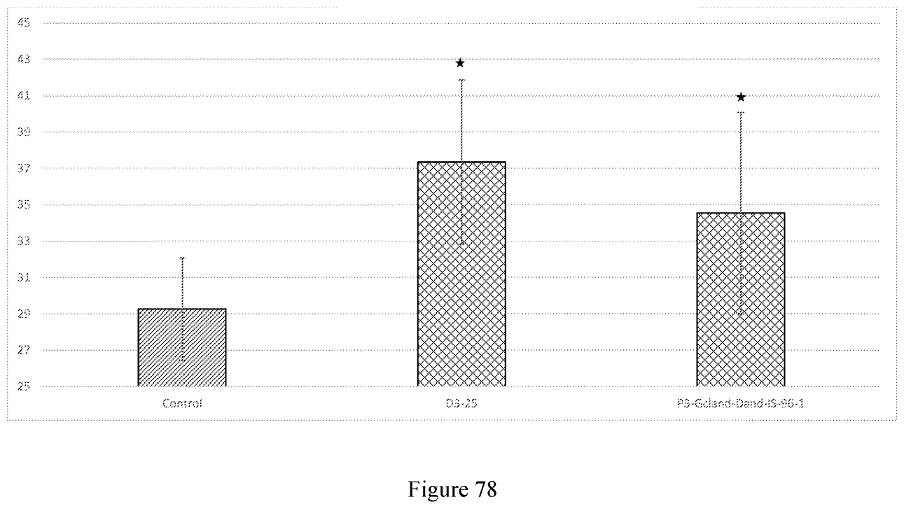

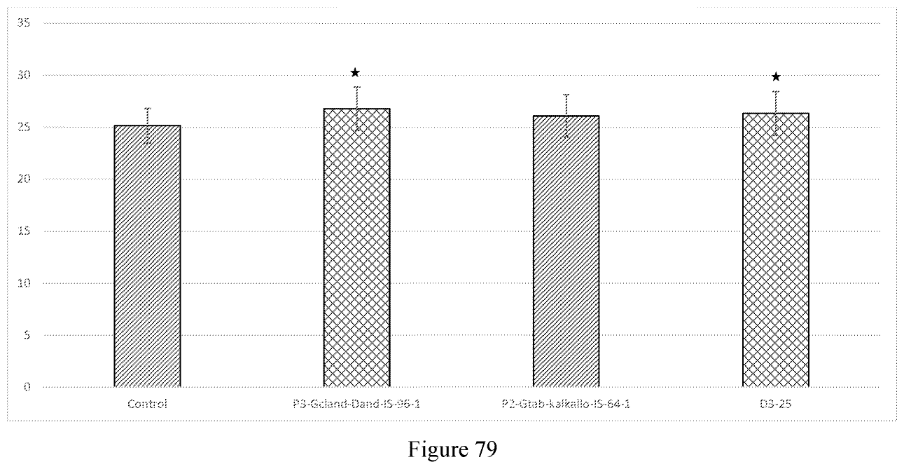

To assess the effect of P3-Gcland-Dand-IS-96-1 on the early plant growth, an assay was established in soybean, using a previously patented Curtobacterium flaccumfaciens I strain D3-25 as a comparison. Curtobacterium flaccumfaciens strain D3-25 and Curtobacterium sp. P3-Gcland-Dand-IS-96-1 were cultured in Lysogeny Broth (LB) overnight at 26° C. The following day seeds of soybean were surface-sterilised by soaking in 80% ethanol for 3 mins, then washed 5 times in sterile distilled water. The seeds were then left to dry in the laminar flow for 15 minutes. The seeds were then soaked in the overnight cultures for 4 hours at 26° C. in a shaking incubator. For control seedlings, seeds were soaked in LB without bacteria for 4 hours at 26° C. in a shaking incubator. Eighteen seeds were planted into 20 cm diameter pots pot at a depth of 1.5 cm per pot in potting mix, with a total of 3 replicate pots per treatment. The pots were arranged in a randomised design. The seedlings that had germinated with two days of each other were selected and the remaining seedlings were removed, leaving eight seedlings per pot. After three weeks of growth, the root and shoot lengths of soybean seedlings were measured. The plants were grown for 3 weeks and then assessed for health (i.e., no disease symptoms), measured and photographed. The lengths of the longest shoot for each seedling were measured. Data was statistically analysed using a one-way ANOVA and Tukey test to detect the presence of any significant difference (p≤0.05) between treatments using OriginPro 2018 (Version b9.5.1.195). Soybean seedlings inoculated in Curtobacterium sp. P3-Gcland-Dand-IS-96-1 had significantly longer roots and shoot than control, with a percentage increase of 18% and 34% respectively (see FIGS. 77 and 78). Soybean seedlings inoculated with previously patented Curtobacterium flaccumfaciens strain (D3-25) had significantly longer roots and shoot than control, with a percentage increase of 28% and 24%, respectively.

### Wheat

To assess the effect of Curtobacterium sp. P3-Gcland-Dand-IS-96-1 and P2-Gtab-NS-kalkallo-IS-64-1 on the early plant growth, an assay was established in wheat, using a previously patented Curtobacterium flaccumfaciens strain D3-25 as a comparison. Curtobacterium sp. P3-Gcland-Dand-IS-96-1, P2-Gtab-NS-kalkallo-IS-64-1, and D3-25 were cultured in Lysogeny Broth (LB) overnight at 26° C. The following day seeds of wheat (cultivar Bob White Red Haplotype) were surface-sterilised by soaking in 80% ethanol for 3 mins, then washed 5 times in sterile distilled water. Eighteen seeds were planted into 20 cm diameter pots pot at a depth of 1.5 cm per pot in potting mix, with a total of 5 replicate pots per treatment. The pots were arranged in a randomised design. The plants were grown for 3 weeks and then assessed for health (i.e., no disease symptoms), measured and photographed. The lengths of the longest shoot for each seedling were measured. Data was statistically analysed using a one-way ANOVA and Tukey test to detect the presence of any significant difference (p≤0.05) between treatments using OriginPro 2018 (Version b9.5.1.195). Wheat seedlings inoculated in Curtobacterium sp. strains P3-Gcland-Dand-IS-96-1 and Curtobacterium flaccumfaciens strain D3-25 had significantly longer shoots than control, with a percentage increase of 6.5% and 4.7%, respectively (see FIG. 79). Wheat seedlings inoculated with Curtobacterium sp. P2-Gtab-NS-kalkallo-IS-64-1 had longer shoots control, with a percentage increase of 3.8% although the difference was not significant.

### Example 7—In planta Inoculations of Soybean Supporting Endophytic Niche and Drought Tolerance Activity of Curtobacterium Bacterial Strains

To assess the ability of Curtobacterium sp. P3-Gcland-Dand-IS-96-1 and P2-Gtab-NS-kalkallo-IS-64-1 to aid drought tolerance, an in planta assay was established in soybean exposed to varying levels of drought. Curtobacterium flaccumfaciens strain D3-25 was used as a positive control. The soybean seeds (cultivar Cowrie) were sterilised as per Example 6. The seeds were then soaked in overnight cultures of the two bacteria for 4 hours at 26° C. in a shaking incubator. For control seedlings, seeds were soaked in LB without bacteria for 4 hours at 26° C. in a shaking incubator. Seeds were planted into 20 cm diameter pots containing potting medium (25% potting mix, 37.5% vermiculite and 37.5% perlite). For each treatment, eighteen seeds were planted at a depth of 1.5 cm per pot in an inch layer of pure potting mix at the top of the pot, with a total of 12 replicates per treatment. The pots were arranged in a randomised design. The seedlings that had germinated with two days of each other were selected and the remaining seedlings were removed, leaving eight seedlings per pot. All treatments were subjected to one of three watering conditions: well-watered (300 mL water every two days), mild drought (150 mL of water every two days), or severe drought (50 mL every two days). After six weeks of growth, the plants were separated into aerial and root tissue, then weighed (wet weight). Data were analysed using OriginPro 2018 (Version b9.5.1.195) as per Example 6.

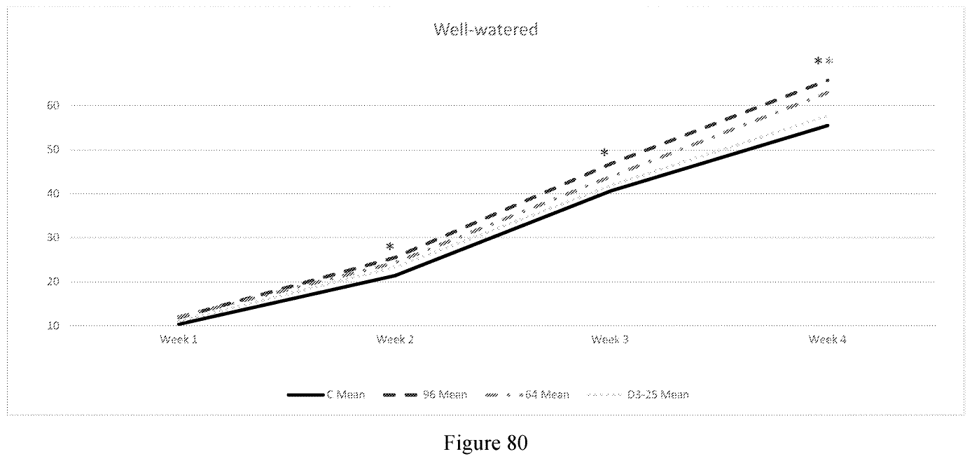

Under well-watered conditions, shoot length was significantly greater in soybean plants inoculated with Curtobacterium sp. P3-Gcland-Dand-IS-96-1 and P2-Gtab-NS-kalkallo-IS-64-1. As shown in FIG. 80, Curtobacterium sp. P3-Gcland-Dand-IS-96-1 significantly increased the shoot length of soybean in Weeks 2, 3 and 4 of growth. By Week 4, soybean plants inoculated with Curtobacterium sp. P3-Gcland-Dand-IS-96-1 had shoots 18.5% longer than control. By Week 4, soybean plants inoculated with Curtobacterium sp. P2-Gtab-NS-kalkallo-IS-64-1 had shoots 14% longer than control.

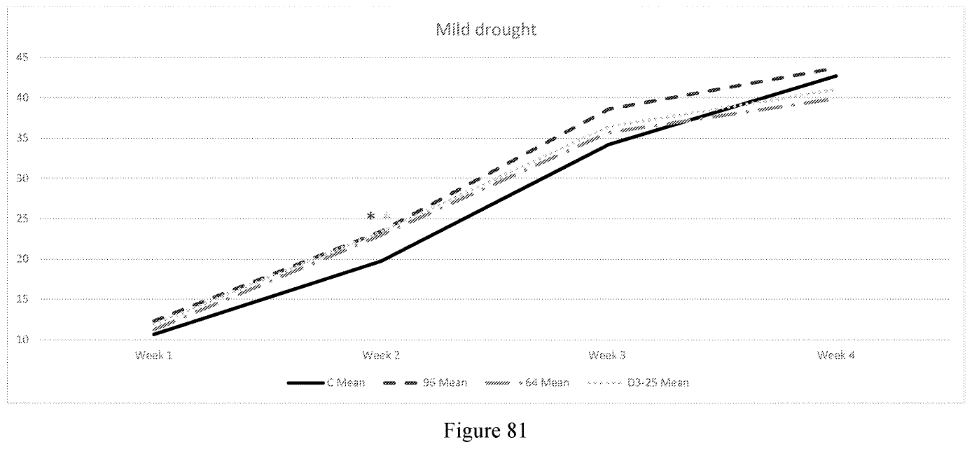

Under mild drought conditions, shoot length was significantly greater in soybean plants inoculated with Curtobacterium sp. P3-Gcland-Dand-IS-96-1 and Curtobacterium flaccumfaciens novel strain D3-25 (FIG. 81). In Week 2 of growth, following the application of mild drought conditions, soybean plants inoculated with Curtobacterium sp. P3-Gcland-Dand-IS-96-1 and Curtobacterium flaccumfaciens strain D3-25 each had shoots 19% longer than control.

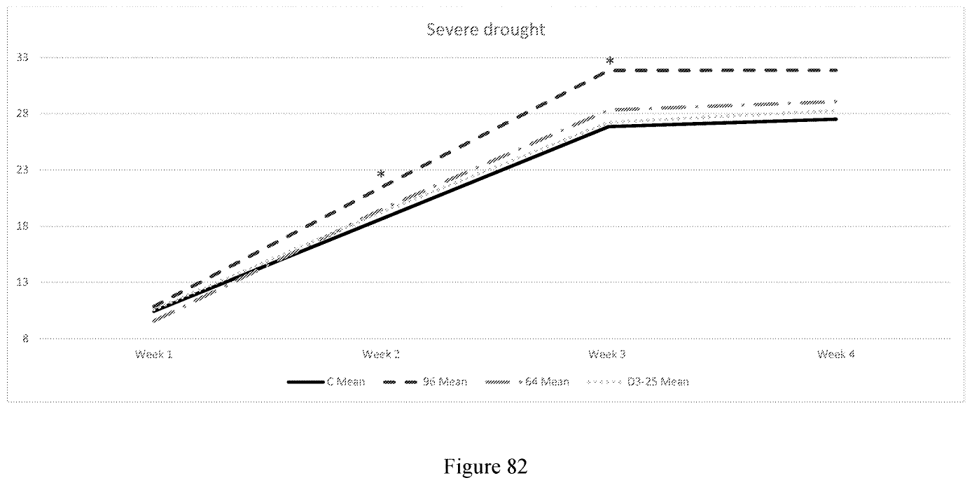

Under severe drought conditions, shoot length was significantly greater in soybean plants inoculated with Curtobacterium sp. P3-Gcland-Dand-IS-96-1 (FIG. 82). In Week 2 and Week 3 of growth, following the application of severe drought conditions, soybean plants inoculated with Curtobacterium sp. P3-Gcland-Dand-IS-96-1 had shoots 15% and 19%, respectively, longer than control.

Under well-watered conditions, root length was greater in soybean plants inoculated with Curtobacterium sp. P2-Gtab-NS-kalkallo-IS-64-1 than in control, although the difference was not significant. Soybean plants inoculated with Curtobacterium sp. P2-Gtab-NS-kalkallo-IS-64-1 had shoots 15% longer than control, although the difference was not significant.

Under mild drought conditions, root weight was greater in soybean plants inoculated with Curtobacterium sp. P2-Gtab-NS-kalkallo-IS-64-1 than in control, although the difference was not significant. Soybean plants inoculated with Curtobacterium sp. P2-Gtab-NS-kalkallo-IS-64-1 had roots 27% heavier than control, although the difference was not significant.

### Example 8—Artificial Seeds Comprising Curtobacterium sp

Embryos are isolated from soybeans and surface sterilised by stirring with 80% ethanol (v/v) for 1 minute. The ethanol is decanted and the embryos rinsed with tap water at least three times. The embryos are then immersed in 15% Domestos® [4.75% available chlorine] that includes 2-3 drops of Tween 20, for 15 minutes with shaking at 150 rpm/min. The embryos are isolated from the sterilisation solution and rinsed several times with sterile Milli-Q water until no trace of foam remains. The sterilized embryos are added to a 100 mL flask with 50 mL of aqueous sodium alginate (5%, 5 g/100 mL) and sucrose grade II (7.5%, 7.5 g/100 mL). The coated embryos are then dropped into a 50-100 mM CaCl₂·2H₂O solution and stirred at 80 rpm for 30 min to form the encapsulated embryos.

### REFERENCES

1. Bulgari, D., Minio, A., Casati, P., Quaglino, F., Delledonne, M., & Bianco, P. A. 2014. Curtobacterium sp. Genome Sequencing Underlines Plant Growth Promotion-Related Traits. Genome Announcements 2(4):e00592-00514. doi:10.1128/genomeA.00592-14.
2. Chen, S., Zhou, Y., Chen, Y., & Gu, J. 2018. fastp: an ultra-fast all-in-one FASTQ preprocessor. Bioinformatics 34(17): i884-i890. doi:10.1093/bioinformatics/bty560.
3. Chun, J., Oren, A., Ventosa, A., Christensen, H., Arahal, D. R., da Costa, M. S., Trujillo, M. E. 2018. Proposed minimal standards for the use of genome data for the taxonomy of prokaryotes. Int J Syst Evol Microbiol 68(1):461-466. doi:10.1099/ijsem.0.002516.
4. de Werra, P., Pechy-Tarr, M., Keel, C., & Maurhofer, M. 2009. Role of gluconic acid production in the regulation of biocontrol traits of Pseudomonas fluorescens CHA0. Appl Environ Microbiol 75(12):4162-4174. doi:10.1128/AEM.00295-09.
5. Löytynoja, A. 2014. Phylogeny-aware alignment with PRANK. In D. J. Russell (Ed.), Multiple Sequence Alignment Methods (pp. 155-170). Totowa, NJ: Humana Press.
6. Lugtenberg, B., & Kamilova, F. 2009. Plant-growth-promoting rhizobacteria. Annual Review of Microbiology 63:541-556.
7. Massela, A. P., Bartram, A. K., Truszkowski, J. M., Brown, D. G., & Neufeld, J. D. 2012. PANDAseq: PAired-eND Assembler for Illumina sequences. BMC Bioinformatics 13(31). doi:10.1186/1471-2105-13-31.
8. Müller, H., Zachow, C., Alavi, M., Tilcher, R., Krempl, P. M., Thallinger, G. G., & Berg, G. 2013. Complete Genome Sequence of the Sugar Beet Endophyte Pseudomonas poae RE*1-1-14, a Disease-Suppressive Bacterium. Genome Announc 1(2):e0002013. doi:10.1128/genomeA.00020-13.
9. Page, A. J., Cummins, C. A., Hunt, M., Wong, V. K., Reuter, S., Holden, M. T., Parkhill, J. 2015. Roary: rapid large-scale prokaryote pan genome analysis. Bioinformatics 31(22):3691-3693. doi:10.1093/bioinformatics/btv421.
10. Paul, N. B., & Sundara Rao, W. V. B. 1971. Phosphate-dissolving bacteria in the rhizosphere of some cultivated legumes. Plant and Soil 35(1):127-132. doi:10.1007/BF01372637.
11. Price, M. N., Dehal, P. S., & Arkin, A. P. 2010. FastTree 2—approximately maximum-likelihood trees for large alignments. PLOS ONE 5(3):e9490. doi:10.1371/journal.pone.0009490.
12. Pritchard, L., Glover, R. H., Humphris, S., Elphinstone, J. G., & Toth, I. K. 2016. Genomics and taxonomy in diagnostics for food security: soft-rotting enterobacterial plant pathogens. Analytical Methods 8(1):12-24. doi:10.1039/C5AY02550H.
13. Richter, M., & Rosselló-Móra, R. (2009). Shifting the genomic gold standard for the prokaryotic species definition. Proceedings of the National Academy of Sciences, 106(45), 19126-19131.
14. Sashidhar, B., & Podile, A. R. (2010). Mineral phosphate solubilization by rhizosphere bacteria and scope for manipulation of the direct oxidation pathway involving glucose dehydrogenase. Journal of Applied Microbiology, 109(1), 1-12. doi:https://doi.org/10.1111/j.1365-2672.2009.04654.x
15. Seemann, T. (2014). Prokka: rapid prokaryotic genome annotation. Bioinformatics, 30(14), 2068-2069. doi:10.1093/bioinformatics/btu153.
16. Vimal, S. R., Patel, V. K., & Singh, J. S. (2019). Plant growth promoting Curtobacterium albidum strain SRV4: An agriculturally important microbe to alleviate salinity stress in paddy plants. Ecological Indicators, 105, 553-562. doi:https://doi.org/10.1016/j.ecolind.2018.05.014
17. Wagner, M. R., Lundberg, D. S., Del Rio, T. G., Tringe, S. G., Dangl, J. L., & Mitchell-Olds, T. 2016. Host genotype and age shape the leaf and root microbiomes of a wild perennial plant. Nat Commun 7:12151. doi:10.1038/ncomms12151.
18. Wick, R. R., Judd, L. M., Gorrie, C. L., & Holt, K. E. 2017. Unicycler: Resolving bacterial genome assemblies from short and long sequencing reads. PLOS Computational Biology, 13(6):e1005595. doi:10.1371/journal.pcbi.1005595.
19. Wick, R. R., Schultz, M. B., Zobel, J., & Holt, K. E. 2015. Bandage: interactive visualization of de novo genome assemblies. Bioinformatics 31(20):3350-3352. doi:10.1093/bioinformatics/btv383.
20. Wood, D. E., & Salzberg, S. L. 2014. Kraken: ultrafast metagenomic sequence classification using exact alignments. Genome Biology 15(3):R46. doi:10.1186/gb-2014-15-3-r46.

1. A substantially purified or isolated Curtobacterium sp. endophyte, wherein the endophyte is capable of conferring a plant growth promotion phenotype to the seed, plant, or part thereof from which it is substantially purified or isolated and/or is capable of conferring a plant growth promotion phenotype to a plant or part thereof to which it is inoculated; wherein the endophyte is from a plant of the Glycine genus.

2. The endophyte according to claim 1, wherein the plant growth promotion phenotype is characterised by enhanced growth of a plant containing and/or inoculated with the endophyte under one or more abiotic stress condition(s) as compared with a plant that is absent the endophyte.

3. The endophyte according to claim 2, wherein the abiotic stress condition includes nutrient, water, and temperature stress.

4. The endophyte according to claim 3, wherein the nutrient stress is characterised as a below normal level of a plant nutrient including nitrogen, phosphorous, potassium, magnesium, sulphur, and calcium.

5. The endophyte according to claim 1, wherein the plant growth promotion phenotype is characterised by a gene encoding phosphate solubilisation, phosphonate solubilisation, phosphate transport, or any combination thereof.

6. The endophyte according to claim 5, wherein the plant growth promotion phenotype is characterised by one or more of the following:
i) gene encoding phosphate solubilisation is selected from the group consisting of ugd, gcd, or gad;
ii) gene encoding phosphonate solubilisation is selected from the group consisting of phnA, phnB, phnC, phnD, phnE, phnW, phnX, ppd, or pepM, and
iii) the gene encoding phosphate transport is selected from the group consisting of pstA, pstB, pstC, pstS, phoP, or phoR.

7. The endophyte according to claim 1, wherein the plant growth promotion phenotype includes at least one gene selected from the group consisting of SEQ ID NOs: 8-18, 20-23, 25-28, 30-33, 35-40, 42-47, 49-54, 56-61, 63-65, and 66-68 or a sequence having at least approximately 95% sequence identity to the full length of at least one of SEQ ID NOs: 8-18, 20-23, 25-28, 30-33, 35-40, 42-47, 49-54, 56-61, 63-65, and 66-68.

8. The endophyte according to claim 1, wherein the seed, plant, or part thereof to which the endophyte is capable of conferring a plant growth promotion phenotype is of the Fabaceae, Poaceae, Apiaceae, Cruciferae, Solanaceae, Cucurbitaceae, Amaryllidaceae, or Brassicaceae families.

9. The endophyte according to claim 8, wherein one of the following applies:
i) the plant of the Fabaceae family is a Glycine species plant selected from the group including G. max, G. clandestina, G. tomentella, G. tabacina, G. canescens, G. latrobeana, G. microphylla, and G. soja; and
ii) the plant of the Poaceae family is a Triticum species plant selected from the group including T. aestivum, T. durum, T. spelta, T. dicoccon, T. dicoccoides, T. turgidum, T. polonicum, T. carthlicum, T. turanicum, T. timopheevii, T. monococcum, and T. boeotictim.

10. The endophyte according to claim 1, wherein the endophyte is substantially purified or isolated from a seed, seedpod, flower, flower bract, leaf, petiole, stem or root of the plant of the Glycine genus.

11. The endophyte according to claim 10, wherein the endophyte is substantially purified or isolated from a seed or seedpod.

12. The endophyte according to claim 1, wherein the endophyte is a Curtobacterium sp. strain denoted P3-Gcland-NS-Dand-IS-96-1, P3-Gcland-NS-Dand-IS-26-1, P2-Gtab-NS-Kalkallo-IS-65-1, P2-Gtab-NS-Kalkallo-IS-64-1, P3-Gtab-NS-Kalkallo-IS-74-1 and/or P3-Gcland-NS-Dand-IS-27-1 as deposited with the National Measurement Institute of 1/153 Bertie St, Port Melbourne, Victoria 3207 Australia on 14 Jun. 2022 with accession number V22/011283, V22/011278, V22/011281, V22/011280, V22/011282 and V22/011279, respectively.

13. A composition including the endophyte of claim 1 and an agriculturally acceptable carrier.

14. The composition according to claim 13 further comprising at least one of components that facilitate the viability of the endophyte, components that assist with inoculation of a seed, plant or plant part and components that assist with transportation or storage of the endophyte.

15. The seed, plant, or part thereof inoculated or infected with one or more endophytes according to claim 1.

16. A seed coated with a composition including the endophyte of claim 1 and an agriculturally acceptable carrier, binder and/or filler.

17. Use of the endophyte according to claim 1 to produce a seed, plant, or part thereof stably infected with the endophyte.

18. A method for conferring a plant growth promotion phenotype to a seed, plant, or part thereof, wherein the method includes inoculating the seed, plant, or part thereof with the endophyte according to claim 1.

19. A method for selecting a plant growth promoting endophyte from a seed or plant of the Glycine genus, wherein the method includes:
a. substantially purifying or isolating one or more endophytes;
b. subjecting the endophyte(s) to an in vitro plant growth promotion assay and/or an in planta plant growth promotion assay; and
c. subjecting the endophyte or selected endophyte(s) to genetic analysis to identify the endophyte species; and
based thereon, selecting an endophyte which is capable of conferring a plant growth promotion phenotype to the seed or plant from which it is substantially purified or isolated and/or is capable of conferring a plant growth promotion phenotype to a seed, plant, or part thereof to which it is inoculated and wherein the selected endophyte is a Curtobacterium sp. endophyte.

20. The method according to claim 19, wherein the selected endophyte is substantially purified or isolated from the seed or seedpod a plant of the Glycine genus.


SEQUENCE LISTING (68 sequences)

SEQ ID NO: 1 - RNA, 1520 nt, Curtobacterium sp.
  source 1..1520: mol_type = rRNA
tacggagagtttgatcctggctcaggacgaacgctggcggcgtgcttaacacatgcaagtcgaacgatgatgcccagcttgctgggttgattagtggcgaacgggtgagtaacacgtgagtaacctgcccctgactctgggataagcgttggaaacgacgtctaatactggatatgatcgccggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacttcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcatggggagcgaacaggattagataccctggtagtccatgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgggttatgccggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggaaggagccgtcgaaggtgggatcggtgattaggactaagtcgtaacaaggtagccgtaccggaaggtgcggctggatcacctccttt

SEQ ID NO: 2 - RNA, 1520 nt, Curtobacterium sp.; identical to SEQ ID NO: 1
  source 1..1520: mol_type = rRNA

SEQ ID NO: 3 - RNA, 1520 nt, Curtobacterium sp.
  source 1..1520: mol_type = rRNA
tacggagagtttgatcctggctcaggacgaacgctggcggcgtgcttaacacatgcaagtcgaacgatgatcaggagcttgctcctgtgattagtggcgaacgggtgagtaacacgtgagtaacctgcccctgactctgggataagcgttggaaacgacgtctaatactggatacgactgccggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagcgaaagtgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcgtggggagcgaacaggattagataccctggtagtccacgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggaaggagccgtcgaaggtgggatcggtgattaggactaagtcgtaacaaggtagccgtaccggaaggtgcggctggatcacctccttt

SEQ ID NO: 4 - RNA, 1526 nt, Curtobacterium sp.
  source 1..1526: mol_type = rRNA
tacggagagtttgatcctggctcaggacgaacgctggcggcgtgcttaacacatgcaagtcgaacgatgatgcccagcttgctgggtggattagtggcgaacgggtgagtaacacgtgagtaacctgcccctgactctgggataagcgttggaaacgacgtctaatactggatatgactgccggccgcatggtctggtggtggaaagattttttggttggggatggactcgcggcctatcagcttgttggtgaggtaatggctcaccaaggcgacgacgggtagccggcctgagagggtgaccggccacactgggactgagacacggcccagactcctacgggaggcagcagtggggaatattgcacaatgggcgaaagcctgatgcagcaacgccgcgtgagggatgacggccttcgggttgtaaacctcttttagtagggaagaagggagcttgctcttgacggtacctgcagaaaaagcaccggctaactacgtgccagcagccgcggtaatacgtagggtgcaagcgttgtccggaattattgggcgtaaagagctcgtaggcggtttgtcgcgtctgctgtgaaatcccgaggctcaacctcgggcttgcagtgggtacgggcagactagagtgcggtaggggagattggaattcctggtgtagcggtggaatgcgcagatatcaggaggaacaccgatggcgaaggcagatctctgggccgtaactgacgctgaggagcgaaagcatggggagcgaacaggattagataccctggtagtccatgccgtaaacgttgggcgctagatgtagggacctttccacggtttctgtgtcgtagctaacgcattaagcgccccgcctggggagtacggccgcaaggctaaaactcaaaggaattgacgggggcccgcacaagcggcggagcatgcggattaattcgatgcaacgcgaagaaccttaccaaggcttgacatacaccggaaacggccagagatggtcgcccccttgtggtcggtgtacaggtggtgcatggttgtcgtcagctcgtgtcgtgagatgttgggttaagtcccgcaacgagcgcaaccctcgttctatgttgccagcgcgttatggcggggactcataggagactgccggggtcaactcggaggaaggtggggatgacgtcaaatcatcatgccccttatgtcttgggcttcacgcatgctacaatggccggtacaaagggctgcgataccgtaaggtggagcgaatcccaaaaagccggtctcagttcggattgaggtctgcaactcgacctcatgaagtcggagtcgctagtaatcgcagatcagcaacgctgcggtgaatacgttcccgggccttgtacacaccgcccgtcaagtcatgaaagtcggtaacacccgaagccggtggcctaacccttgtggaaggagccgtcgaaggtgggatcggtgattaggactaagtcgtaacaaggtagccgtaccggaaggtgcggctggatcacctccttt

SEQ ID NO: 5 - RNA, 1520 nt, Curtobacterium sp.; identical to SEQ ID NO: 3
  source 1..1520: mol_type = rRNA

SEQ ID NO: 6 - RNA, 864 nt, Curtobacterium sp.
  source 1..864: mol_type = rRNA
atgacggccccggactcggtgcagaccgacggtgccgcgggcacggtcgagcagccgtcgctcgcgccggtccggctcgacgccgccgtcgccgcacgcggcctcgcgaagtcacgcaccgctgccgcgaagctcatcctggacggccgtgtcagcgtggacggcgctccggtcgtcaaggcatcgacgccggtcaccgcggacacccggatcgtggtcgacgccggcgacgagtgggtgtcgcgtgcggcgctcaagctcgtcggcgcgctcgacaccttcggcgtggacccgtcgggtcgcgtcgcgctggacgtcggcgcgagcacgggcggcttcacgcaggtgctcctggcccgcggagcacgacgtgtggtcgcgctcgacgtcgggcacgggcagctcgacccggtcgtggggctcgacgaccgcgtcaccgtggtcgagggcgtcaacgccaggaacctgacggccgccgactacctggcactcgaccccgtcgccggagagaccagcctggtcgtcggggacctctccttcatcagcctgcggatcgtcctgcccgcactcgtcgaggccgtgccggcggacgagtacgtcctgctcatcaagccgcagttcgaggtcggtcgcggcggtgtccgtgagggcatcgtgcacgacgccgacctccgcaacgacgcgctgatgaacgtgctgtgggccgcgtgggacctcggcttcgggaccgccggcatcgcggcctcgccgatcctcggcacccacggcaaccacgagtacgtcgcgcgtctccagcggggcgcagggtccaatccgacagaatggatcgaccgagccacggaactggcggaaggaaccccatga

SEQ ID NO: 7 - DNA, 1428 bp, Curtobacterium sp.
  source 1..1428: mol_type = genomic DNA
atgagcgagtccaagaagtcgaccgcatcggccgacacccgccccgcacccgtcatcagcgtcatcggcaccggctacctcggcgccacccacgcggccgccatggccgagatgggcttcgagaccatcggtgtcgacatcgacccgtcgaagctcgccgcactgtccgccggtgaggtcccgttcttcgagcccgggctgcccgagctcatcacgaagcacgtcgccagcggcaagctccggttcacctccgacatcgcctcggccgtcgccgccgcggacgtccacttcgtctgcgtcggcaccccgcagaaggccgggtcgcacgccgcgaacctggcctacgtcgagagcgccacccgtggcgtcgccgagaacctcacccacccgggcctcatcgtcggcaagtcgacggtcccggtcggcaccgcatcgcgactccgcggcatcgtccgcgagttcacgcccgccgggatcgacgccgagctcatctggaaccccgagttcctgcgtgagggcaaggccgtcgaggacaccctgcacccggaccgtctggtctggggtggagcctccgaggccgccgacgccgtgatccgcgaggtctacgccgcacccatcagcgagggcaccccggtcatcaccaccgacctcgccaccgcggagctcgtcaaggtgagcgccaacgcgttcctcgccacgaagatctcgttcatcaacgcgatctccgagatgtgcgccatcaccggcgccgacgtctccacgctcgccgacgccctcggccacgacgcccgcatcggccggaagttcctgaacgccggcctcggcttcggcggcggctgcctgccgaaggacatccgggcgctcatgtaccgcgcgaacgagatcggcgcgggccaggtcgtcggcctcatgcagcaggtcgacgagatcaacatggggcagcgccagcgggtcatcgacatggcgatcgactcggccgggggatcggtgctcaaccgtcgcgtggccgtcatcggtgccgcgttcaagcccctgaccgacgacgtccgggactccccggcgctgaacgtcgccgccgcgctgcacctccgcggcgcccaggtgacgctctgggaccccgaggcgatggagcccgcgaagcgctcgttcccgacgctgagctacgccgcctcgatgacggacgcgatcgagggcgccgacgtcgtcctcgtgctcaccgagtgggacgacatcatcaccgccgacccggtcgccctcggcgacctcgtcgggcgccgcgtcgtcatcgacgcgcgcaactgcctcccggtcctcgactgggtgaaagcggggtggaccgtcagttccctgggacgtcccacgccggtcacgggtgcacccgtgagcgtcgcagcggggacgaacgacgcgttggccacgagccgctag

SEQ ID NO: 8 - DNA, 1308 bp, Curtobacterium sp.
  source 1..1308: mol_type = genomic DNA
gtgcgtatctctgtcatcggctgcgggtacctcggtgccgtgcacgccgcctccatggccaaactcggacacgaggtcgtcgcggtcgacgtcgacccggtgaagatcgagcggctcgccgccggcgacgccccgttcttcgaaccgggactccccgagatcctcgcggaggcgatcgggtccgggcggatccgcttcaccacggacatgtccgaggtcgccggggcccgcgtgcacttcgtcgccgtcggcaccccgcagggcccgaccggcgccgccgacctgacgtacgtcgacgccgccgtggccgcgctgacgcagtacgtggccgcggacgagttcgtcgtcggcaagtccaccgtccccgtgggcaccgccgcccgcctggcggcgtccctgtccgaggccgtgcccggcgcgacgctcgtctggaaccccgagttcctccgcgagggcttcgccgtgcaggacaccatctccccggaccgcctggtctacggcgtgccgtcgggtgccgcgggcgagtccgccgtcgccgccctcgacgaggtctacgccacgaccctggccgaggggaccccacgcctggtggtcgacctggccaccgccgagctcgtcaaggtcagcgcgaacgcgttcctggcgacgaagatctccttcatcaacgcgatggccgagatcgccgaggtcgtcggtgccgacgtcacggcgctcgccgacgccatcgggcacgacgcccgcatcggtcggaagttcctgaacgccggcctcgggttcggcggcgggtgcctgccgaaggacatccgcgcgttccaggcccgggcgaccgagctcggcgtgggggagtcgctcgcgttcctggccgacgtcgacgccatcaacctgcggcgtcgcgcgcacgtcgtcaccctcgccgaggagctgctcgggggcgacgtcaccggcaagcacgtggccgtcctcgggctcgccttcaagccgaactcggacgacgtccgcgactccccggcgctcgacatcaccgcacggctgcacgagctcggcgccgtcgtgtcggcgtacgaccccgaggcgaacggcaccgccgcccgcatccaccccgaggtccactacgtcgacatcgcggacgaggcactccgtggcgccgacctggtgctcgtgctgaccgagtggaacgcgttccgcgagctcgacccgaccgtggtcgccggtctcgtgcagaacccggtcgtggtcgacggccggaactgcctggaccgcgacgcctggaccgccgccggcttcacggtgcgcgggatgggccgctag

SEQ ID NO: 9 - DNA, 1308 bp, Curtobacterium sp.; identical to SEQ ID NO: 8
  source 1..1308: mol_type = genomic DNA

SEQ ID NO: 10 - DNA, 1308 bp, Curtobacterium sp.
  source 1..1308: mol_type = genomic DNA
gtgcgtatctccgtcatcggctgcgggtacctcggtgccgtgcacgccgcttccatggccaagctcggacacgaggtcgtcgccgtcgacgtcgaccccgccaagatcgaacggctcgccgccggcgacgcccccttcttcgagccgggactgcccgagatcctgtccgaggcactggcctcgggccgcatccggttcaccacggacatgtccgaggtcgccgggtccaccgtgcacttcctcgccgtcggcaccccgcagggcccgaccggcgccgcggacctcacctacgtcgacgctgccgtcgccgccctgacgccgcacctgtcggcgggggagttcgtcgtcggcaagtcgaccgtcccggtcgggaccgccgcgcggctcgccgccgacgtggagcgggccgtcccgggcgcgacgctcgtctggaacccggagttcctgcgcgagggcttcgccgtgcaggacacgatctcgcccgaccgcttcgtgtacggcgtccccgagggcgaggccggcgagcgtgccgtcgccgcgctcgacgaggtctacgcgcaggcgctcgccgcggggacgccccgactcgtggtgaacctgcccaccgcggagctcgtgaagatcagcgcgaacgcgttcctcgcgacgaagatctccttcatcaacgcgatggccgagatcgccgaggtcgccggcgccgacgtcaccgcgctcgccgacgcgatcggccacgacgaccggatcggccgcaagttcctcaacgccggcctcggcttcggcggcggctgcctgccgaaggacatccgcgcgttccaggcccgcgccgacgagctcggtgtgggcgagtccctcgccttcctcgccgacgtcgacgccacgaacctgcgccgtcgcgcccacgtggtcgacctcgccgtcgagctgctcggcggctcggccgagggcaagcgcgtcgccgtcctcgggctggcgttcaagccgaactcggacgacgtccgcgactccccggcgctcgacatcgcggcccgcctgcacgagctcggcgcgaccgtcgccgctcacgaccccgaggcggtcggcaccgcacgtcgactgcacccggagctcgagtaccgcggatcggcgtccgaggcgctcaccggcgcggacctcgtgctcgtgctgaccgagtggacggcgttccgcgagctcgacccgaccgaggtcgcgtcgctcgtggcgaacccggtcgtcgtcgacggccgcaactgcctggaccgcgaggcgtggaccgccgccgggttcacctaccgcggtatggggcgctga

SEQ ID NO: 11 - DNA, 1308 bp, Curtobacterium sp.
  source 1..1308: mol_type = genomic DNA
gtgcgaatctctgtcatcggctgcgggtacctcggtgccgtgcacgccgcatccatggccaagctcgggcacgacgtcgtcgccgtcgacgtggacccggtcaagatcgagcggctcgcggcgggcgaggccccgttcttcgaacccggtctccccgagatcctgtccgaggcgctcgcgtccgggcgcatccgcttcacgaccgacatggccgaggtggccggtgcccgcgtgcacttcctcgccgtcgggaccccgcaggggccgaccggcgccgcggacctcacctacgtcgacgccgcggtcgccgggctgatcccgcacctgtcggcgggcgagttcgtcgtcggcaagtcgacggtgccggtggggaccgccgcgcgcctcgcggccgaggtcgacgccgcggtgcccggtgcgacgctcgtctggaaccccgagttcctccgcgagggcttcgcggtgcaggacacgatctccccggaccgcttcgtgtacggggtgccggagggcgaagccggtcaggcagcggtggacgccctcgacgaggtctacgcggaggcactcgccaccggcacaccccgcctcgtcgtgaacctccccaccgccgagctcgtgaagatcagcgcgaacgcgttcctcgcgacgaagatctcgttcatcaactcgatggccgagatcgccgaggtcgccggggccgacgtcactgcgctcgccgacgccatcggccacgacgcccgcatcggccggaagttcctgaacgccggcctcggcttcggcggcgggtgcctgccgaaggacatccgcgcgttccaggcccgtgcgaacgagctcggggtgggggagtcgctggcgttcctcgccgacgtcgacgccaccaacctgcgtcggcgtgcgcacgtcgtcgagctcgcgcaggagctcctcggcggtgcggtgcagggcaagcgcgtcgccgtgctcggcctggcgttcaagccgaactcggacgacgtccgcgactcgccggcgctcgacatcgcggcgcggctgcacgagctcggcgcctcggtcgccgcgtacgacccggaggccgtcgtcaccgcgcgtcgaatccgcccggagctcgagtacgtcgagcgagcgacggacgcgctccgcgacgccgagctcgtgctcgtgctgacggagtggaacgagttccgcgagctcgacgctgccaccgcggccgcgctcgtgtcggtgccggtcgtcgtcgacggccgcaactgcctcgaccgcgaggcctgggtcgccgccggcttcacctaccgcgggatggggcgctga

SEQ ID NO: 12 - DNA, 1308 bp, Curtobacterium sp.; identical to SEQ ID NO: 10
  source 1..1308: mol_type = genomic DNA

SEQ ID NO: 13 - DNA, 1422 bp, Curtobacterium sp.
  source 1..1422: mol_type = genomic DNA
atgaccgctcagaagcactccgacaaggacggccgtcccgcgcccgtcatcagcgtcatcggcaccggctacctcggtgccacccacgcggccgccatggccgagatgggcttcgagaccatcggcgtggacgtcgacccggcgaagctcgccgcgctgtccgccggcgaggtcccgttcttcgagcccggcctgcccgagctcatcacgaagcacgtggccagcggcaagctccgcttcacctcggacatcgcgtccgccgtcgcggctgctgacgtgcacttcgtctgcgtcggcacgccccagaaggccggctcgcacgccgcgaacctcgcctatgtcgagagcgccacccgcggcgtcgccgagaacctgacccaccccgggctcatcgtcggcaagtcgacggtcccggtcggcacggcggcacgactgcgcggcatcgtccgcgagttcgcccccgtcggcatcaccgccgagctcatctggaaccccgagttcctccgcgagggcaaggccgtcgaggacaccctgcacccggaccgcctggtctggggtggcgcgtccgaggcagccgacgccgtgatccgcgaggtctacgccgcaccgatcagcgagggcaccccggtcatcacgacggacctcgccaccgccgagctcgtcaaggtcagcgcgaacgcgttcctcgccacgaagatctcgttcatcaacgcgatctccgagatgtgcgccatcaccggtgccgacgtcaccacgctggccgacgccctcggccacgacgcccgcatcggccggaagttcctcaacgccggcctcggcttcggcggcggttgcctgccgaaggacatccgggcgctcatgtaccgcgcgaacgagatcggcgccggccaggtcgtcggcctcatgcagcaggtcgacgagatcaacatgggccagcgccagcgggtcatcgacatggcgatcgacgctgccgggggatcggtcctgaaccgccgcgtggccgtcatcggcgcagcgttcaagccgctcaccgacgacgtccgcgactcgccggcgctcaacgtcgccgcggcgctgcacctccgcggcgcccaggtcacgctctgggacccggaggcgatggagacggccaagcgctcgttcccgacgctgagctacaccgcgtccatgaccgaggcgctcgagggtgccgacctcgtcctcgtcctcaccgagtgggacgacatcatcagcgccgacccggtcgcccttggggacctcgtcggacgtcgcgtcgtcatcgacgcgcgcaactgcctcccggtcaccgactgggtcaaggcggggtggtccgtccgttccctgggacgtcccacgccggtcgcgggtgcacccgtgagcgttgctgcagggtccaacgacgtgctggccaccagccgctag

SEQ ID NO: 14 - DNA, 1122 bp, Curtobacterium sp.
  source 1..1122: mol_type = genomic DNA
atgcgcacccggaccgcagcacccctcgcatcgctcgccctgctcgtcctgacgctcgccggctgctccgccggtccggacgcggaggagggccgttcctcgcggggcagtgcgacggctcccccggcgccggacgacctggcgaccggtgaggtcgtccccacgggtgccgtgacggacgtcgcgaccgacctcgaggcgccgtggtccgtcgtgctggtcgacgacaccccgctggtgagcctccgcgactccggcgacgtgctcgagctcgcgaaggacgggactccgcgcaccgtcggcacgatcgagggcatccgacacggcggcgagggcgggctgctcggcctggcggtgcacgacgacgacgagctctacgtctactcgaccggcgaggacggcaaccgggtcgagcgcttcccgctcaccggcgatgccgggtcgtacgggctcggcgacccgacgaccgtcatcgaggacctgcccgcgaacagcttccacaacggcggtcggatcgccttcggaccggacgacatgctctacgtgaccgtcggtgacgccggcgaccggtccgaggcccaggaccgcagctcgctcgccggcaagatcctgcgcgtcacgccgaccggtgccgtgcccgacgacaacccgttcgacggctccccggtgtggtccctcgggcaccgcaacgtgcaggggctcggctgggcggacgacggcacgatgttcgcgtcggagttcggggagaacgccttcgacgagctcaacgtcatcgagcccggaagtgactacggctggcccgaggtcgagggcaggggcggcgccgaccagggcttcgtcgacccggtgcagcagtggtcgacggacgaggcgagcccgagcggcctcgccgtcgtcgacgacaccgtcttcatcgcgaacctgcagggccgggtgctccgctcggtgcccgtggccgacccgtcgacctcgcgggagtggttcagcgacgagtacgggcgcatccgcaccgtgctcgagggccccgacgacacgctctggttcgtcacgaacaacaccgacggtcgcgggtcacccggtgacggcgacgaccgggtcctgtcggtgggcctgtccgactga

SEQ ID NO: 15 - DNA, 1122 bp, Curtobacterium sp.; identical to SEQ ID NO: 14
  source 1..1122: mol_type = genomic DNA

SEQ ID NO: 16 - DNA, 1194 bp, Curtobacterium sp.
  source 1..1194: mol_type = genomic DNA
atgtcaacgtcgatcaccgacacgcccctgctcgaatccgacttctaccggttccaggaggggctcacggcccgtgagcgggagtcgctcgggcagctgcgcgcgtacctcgaggccgaggtccgcccgatcgccgacgggtactgggcccgcgccgagttcccgatgcagacgatcgcgccgctcgccgagctcggcatgtacggaccgagcatcccgctcgtgcggcagttcgagaactccgccgtgtaccgcggctgggccgcactcgagctcgggcgcgtggacgccggtgtgtcgaccttcatcggcgttcagtccggcctcgcgatgaactcgatcgccgtgggcggcagcgacgagcagcagcaggagtggctgccccgcatggcccgcggcgaggtcgtcggcgccttcgggctgaccgagccgctgtccggtagtgactcggcacgggggctccgcacgaccgcccgacgcgacggtgactcgtgggtgctcgacggcgagaagcgctggatcggcaacgccaccttcgcggacgtcgtcgtcatctgggcgaaggacgtcgccgacgagcaggtcaagggcttcctcgtgacgacggacacacccggcttcaccgccacgaagatcgaggacaagatcgccctgcgcacggtgcagaacgccgacatcgtgctcgacggcgtccgggtccccgagtcgcgccggctgcagcgagcggactcgttccgcacgacggccgcggtgctccgcctgacccggaccgaggtcgcgtggcaggcgatcggcatcgcgatcggcgcctacgaggccgcgctcgactacgcgcggaaccggatccagttcggcaagccgatcgccgcgcaccagatggtgcaggacctgctcgtgaagtcgctcatgaacatcaccgcgtcgatcgcgctgtgcacccaggcgtccgcgatgcaggaccagggcgtcggcgccgacgagcactcggcgatggcgaaggcgttcgccaccgcgaagatgcgcgagactgtcggctggtgccgcgaggtccagggcggcaacggcatcgtgctcggcaagggtgtcgcgcgcttcttctccgatgccgaggcgatctactcctacgagggcacgcgcgaggtgaacacgctcatcgtcggacgggcgatcacgggccaggccgcgttcgtctga

SEQ ID NO: 17 - DNA, 447 bp, Curtobacterium sp.
  source 1..447: mol_type = genomic DNA
atgacctcgcacctgagcccctacctgggcttccgcgacgacgcccgccaggcgatggagttctaccagtcggtgttcggcggtgaactgagcctcgggaccttcggcgagatgcacgcttccgacgacccggccgaggtcgacaagatcatgcacggacagctggtgacgccgaacgggttcctcctcatggggtccgacaccccgagcggcatggagcgttcgtcgacgagcaacatctccctgtcggtgtccggggacgacgccgaggacctccgccggttcttcgccgcgctgtcggagggcggcacggtcatcgagccgctgaccgtcgcaccgtggggagacgagttcggcatgctgaccgaccggttcggcgtgacctggctcgtgaacgtcaccggagccggggccgcttcctcgcccgaggcagacgtccacgcctga

SEQ ID NO: 18 - DNA, 388 bp, Curtobacterium sp.
  source 1..388: mol_type = genomic DNA
atgacctcgcacctgagcccctacctgggcttccgcgacgacgcccgccaggcgatggagttctaccagtcggtgttcggcggtgaactgagcctcgggaccttcggcgagatgcacgcttccgacgacccggccgaggtcgacaagatcatgcacggacagctggtgacgccgaacgggttcctcctcatggggtccgacaccccgagcggcatggagcgttcgtcgacgagcaacatctccctgtcggtgtccggggacgacgccgaggacctccgccggttcttcgccgcgctgtcggagggcggcacggtcatcgagccgctgaccgtcgcaccgtggggagacgagttcggcatgctgaccgaccggttcggcgtgacctggc

SEQ ID NO: 19 - DNA, 837 bp, Curtobacterium sp.
  source 1..837: mol_type = genomic DNA
atgacgaaccacgcaccggtcggtaccccggccgtcgagatcaccggcctgacgaagcgcttcggcgacacgacggcgttgcgcgacgtgtcgctgcgcgtcgggcagggcgaggtcgtcgtgctgctcgggctgtcgggctccggcaagagcaccctgctccggcacgtcgacgcactcgagctcccgacgtccggcagcgtccaggtggacgggaccgaggtgcaccgtctgaccgggcgccgtctgcgcgagctgcggagtcgcgtcgggttcgtgttccagcagttcgaactcgtgccggcgctcaccgtgctcgagaacgtgctcaccggagcgctcgcgaccctccgcggcccgcggctcggcctctggtcgtacccgcgcggcctccgtcgcaccgcactcgggcacctcgaccgcgtcgggctgctcgaccgcgcgtaccagcgtgccgacaccctgtcgggcgggcagcagcaacgcgtcgcgatcgcgcgggccctcatgcaggacccgacggtgctcctggccgacgaaccggtcgcctcgctcgacccggagtccagcgggcaggtcatgcgcctcatccgcgagatcgccgccgaccgcgggctgacggtgctctgcagcctgcaccaggtcgacctggccctgtcgtgggcggaccggatcatcgggctccggcacggcgaggtcgtgctcgacacccggaccgacggcctcggcaaggcggaggtcatggagatctacgggcaggtcgcgacgagcaccgacgagttcgccacgatcgaggccgagctgcacgcactcgacacgggggagcggacctcgtga

SEQ ID NO: 20 - DNA, 846 bp, Curtobacterium sp.
  source 1..846: mol_type = genomic DNA
atgcagcacacgacgacctcccccgtgatctcggtcaccggggtcacgaaggacttcggcaccacgagggccctcgacggcatcgacctgaccgtcgagcagggggaggtcgtcgtgctgctcgggctgtccggctccggcaagtcgacgctgctgcggcacgtcaacgggctcgagctcccgacctccggttccgtccgggtgctcggcgaggagctcaccacgctccgaccgcgcgccctccgcacgatgcgcggccgggtcgcgatggtcttccagcagttcgaactggtgccggcgctgacggtgctcgagaacgtcctgaccggggcgctgtcccggctgtcgggtccgcggctcggcatctggtcgtacccgcgggccgtccggaccgcggcgctgacccacctggaccgcgtggggctgctcgagaaggcctaccagcgtgccgacgagctctccggcggccagcagcagcgcgtcgcgatcgcccgggcactcatgcaggacccggaggtgctgctcgccgacgagcccgtcgcgtcgctcgacccggagtcgagtgcgcaggtgatgcggctcatccgggagatcgccgccgacgacggcctgacggtcgtctgcagcctgcaccaggtcgacatcgcgctcggctggggcgaccgcatcgtcgggctccggcacggcgaggtcgtcctggacctgccgacggccggcatcagcaaggcccaggtcatggagatctacggccgcgtgagcacgtcctcggccgagatgcgggtcatcgagacggagctcgccggcgtcggcctgctcgacccggcacgcgcagcgggcgaccgatga

SEQ ID NO: 21 - DNA, 846 bp, Curtobacterium sp.; identical to SEQ ID NO: 20
  source 1..846: mol_type = genomic DNA

SEQ ID NO: 22 - DNA, 849 bp, Curtobacterium sp.
  source 1..849: mol_type = genomic DNA
atgacgtaccacgaccaccagcacgaccaggcgccggcagtggcgatcaccgccctgacgaagcgcttcggtgacacgaccgccctcgacgacgtgtcgctcaccgtccagccgggcgaggtcgtcgtcctcctcggactgtccgggtccggcaagtccacactgctccggcacgtggacgccctcgaggtcccgacctccggctccgtccgggtcggcgggaccgccgtgcacgacctgtccggacggcggctccgtgacctgcgtcggcaggtcggcttcgtgttccagcagttcgagctcgtgcccgcgctgacggtgctcgagaacgtcctgaccggggcactcgccaccctgcgggggccgcggctcggcctgtggtcctacccgcgtcgactgcggcaccgcgcgctcgcacacctcgaccgtgtcggactgctcgaccgcgcctaccagcgcgcggacaccctgtcgggcgggcagcagcagcgcgtcgcgatcgcccgggcgctcatgcaggacccctccgtcctgctcgccgacgagccggtcgcgtcgctcgaccccgagtcgagcggtcaggtcatggcgctcatccgcgagatcgccgccgaacgagggctgacggtgctgtgcagcctgcaccaggtcgacctcgcgctgtcctgggccgaccggatcgtcggactccggcacggcgaggtcgtcctcgacacccgcaccgagggactcgacaaggcgcaggtcatggagatctacggccaggtcgcgacgtcgaccgacgagttcgcggccgtcgaggcactgcgcgacgcgccggcccacgccacgagcgggcagcgggcatga

SEQ ID NO: 23 - DNA, 846 bp, Curtobacterium sp.; identical to SEQ ID NO: 20
  source 1..846: mol_type = genomic DNA

SEQ ID NO: 24 - DNA, 942 bp, Curtobacterium sp.
  source 1..942: mol_type = genomic DNA
atgcccatctccgcacgcaagtcgctcgtcgcgctggccggcgccctggccctcgtcgccacgctcagctcctgctccgcaggctccaccgccgcgaccggtgacggcagccccgcaggcggctccgacgccgtgcagtacgccgcggacgagaacaccatcgtgttcggcgtggtccccgactccgtggacaccgagacgaactaccagccgctgatggactacatcgcgaaggagaccggcaagaaggtcgagtaccacgagtcgaccgactacgccgcgctcatcgaggcgtccgtcgcgggcaagatcgacgtcgcgtcgttctccgggttcacctacgtcacggcgaagaacaacggcgcgaagatcaccccgatctcctccatcatcaccgccgaggggcagaagcccgggtactactccgaggccatcgtgccgaaggacagtgacatcgactcgctcaaggacttcgctggcaagaaggtctgcttcgtcgacccctcgtcgacctcgggctacctgttcccgagctacaacctgctcaaggcgggcgtcgacccgaagaccgacgtcaccccggtgttcgccggcaagcacgacgtcagcgtccagaaggtcgcggcgggcaccgagtgcgacgccgggttcgccgaggacagcgaggtcgagaagaacgacgacgtgcgcgtcgtcgcgaagacgatggtccccggcgccccgatcgtcgtctcggacaccctgccgtcggccgagaaggagacgctgacgaaggcgctgtccgacgtcaccatcgaccagatcatcgacgccggtgtcgacagcgccgacagcgacggcttccgcagcgtcttctacggcaccaagcccgtcgacgacgagtactacgacctgatccgcgacatctgcgagacgacgaagtcgaccacctgccaggcctga

SEQ ID NO: 25 - DNA, 981 bp, Curtobacterium sp.
  source 1..981: mol_type = genomic DNA
atgcatctgtcccgaagcacgcgcatcggcatcgtcggctctgtcctgaccctcgcggtggtcgccctgaccgggtgctccgcgagcagcaccgccgccaccggcgacggcacggccgccgccgcctcgtccggcacctgggccaaggcgaagggcacgctcgtgttcggcgcggtccccgaccaggccggctccgacgcgaacaacaagccgctcgaggagtacatcgccaagaagaccggctaccaggtcgagtactaccccaccgccgactacacggccctcatcgcagcagcggtcgccggcaaggtcgacgtgatgtcctccggcgggctgcagtacgtgcaggccacgaacaagggcgcgtcgctcgcgccggtggccgcgacgctcagctcccccgacgtgaaggaccccgggtactactccgaggccatcaccaaggccggctcgtcggtcacctcgctgaagcaggcgaagggcaagacggtctgcttcgtcgacccgaactcgacctcgggcttcctgttcgggctgtaccagctgaacaaggccggggtggacgtctcgacctcgggcacggacgccagcggcaacccgaagttcaccgcattcaccgcgtacttcgccggtgcccacgacaagtccgagcaggccgtggcatcggggcagtgcgatgtcgggttcgccgaggactcgatcgcggagcccgccgccaagaagggcgacatcacggtcctcggcaagcagtacgtgcccggcggcccgctctcgatctcgacgaagctgccgaaggacgtgcagacgaagctcgccggcgtcctggcgggcgcatcgccggaggccatcaagaaggccgggatcacgatgacggacggcttcaccgaggggtacttcggcgcccaggccgaggacgcgtcgtactacaagtcgatccgcgacctctgctccaccatcccgagcgccgactgcgccaagtga

SEQ ID NO: 26 - DNA, 981 bp, Curtobacterium sp.; identical to SEQ ID NO: 25
  source 1..981: mol_type = genomic DNA

SEQ ID NO: 27 - DNA, 930 bp, Curtobacterium sp.
  source 1..930: mol_type = genomic DNA
atgcccatccccgccctcaagcccctggtcgcactcgccggcgccctggtgctcgccgccacgctcagctcctgctccgccgggtcgacggcggcggccggcagcgacgagaccgcgtccacgaagtacgccgccgacgccgacaccatcgtcttcggcgtcgtgcccgactcggtcgacaccgagaccaactaccagccgctgatggactacctcgcgaaggagaccggcaagaaggtcgagtaccacgagtcgaccgactacgccgccctcatcgaggcctcggtcgccggcaagatcgacgtcgcctcgttctccggcttcacctacgtgacggcgaagaacaacggcgcgaagatcacgccgatctcgtcgatcatcaccgccgaggggcagaagcccggctactccagcgaggcgatcgtcccgaaggacagcgacatcacctcgctgaagggcttcgagggcaagaaggtctgcttcgtggacccgtcctcgacgtccggctacctcttcccgagctacaacctgctgaaggccggcatcgacccgaccaaggacgtcacgccggtcttcgccggcaagcacgacgtcagcgtgcagaaggtcgccgcgggcaccgagtgcgacgccggcttcgccgaggacagcgaggtcgagaagagcaacgacgtgaaggtcgtcgcgaagacgatggtgcccggcgcgccgatcgtcgtgtcagacaccctgccgcaggccgagaaggacaccgtcacgaaggcgctgtccgacgtcaccatcgacgacatcatcgccgcgggtgtcacgagcgccgacagcgacggcttccgcagcgtcttctacggcacgaagccggtcgacgacgggtactacgacctgatccgcgacatctgcaagacgaccaagtcgagcacctgccaggcatga

SEQ ID NO: 28 - DNA, 981 bp, Curtobacterium sp.; identical to SEQ ID NO: 25
  source 1..981: mol_type = genomic DNA

SEQ ID NO: 29 - DNA, 1746 bp, Curtobacterium sp.
  source 1..1746: mol_type = genomic DNA
gtgacgacgctcgacacccgacccgggaccgtggcggcgctcgccgaggacgtggcccgtcgggcaccccgccgccgggtctcgccggagcgcacggcggcggtgctgaccctcgtcgccctcggcgtgctggcgtgcgtcgcggtcgcgcgggtcgacatctcgatcccgaacatcatcgcgtcgtggtcgaacgccgagcacttcttccagcgtgtggggaccgtccggttcccggccgccccggtgctgctgcagcagaccgcgctcaccgtcgggctcgtcctgctcggcaccctgctcgccgccgcgctgagcgtccctgtcgcgtacttggcggcagcgaacaccagtccgggccgcgggtgtgtggccgcagcgcggttcgtcagcgtcctgacccgggccgtgccggacgtcgtcctcgcgatggtgttcgtcctggtcttcacgatcggtccgctgcccgggatcctggcgatcggcatccactccgtcgggatgatctcgaagctcttcgccgacgccatcgagcagatcgacgaagggccgcgcgacgcgatccgggcagccggggggagccgtgcgcagcagttcaccgccggggtgctcccgcaggtgctgccgtcgtgggtcgcgacgatcctgcaccgcaacgacatcaacctgcgcgggtcggtcatcctcgggtacgtcggcatcgtcggcctcggccgcgagatgtccttcgcgttcaagtcgctcgactactcactcggcatcgggtacgcgatcgtcatcttcggcctgtgcgtcctgatggagatcgtctccagctcggtgcgcgccgccatgctcggcgtggccccggccgggcgcggcatcggtgccacggctgcccgtcgatggatgcgtcgacgggaccggcagcgagttgaggccgcaccgtccacgcggcgaccggtcgccgcgacggtcgagggcaccctccgccgcccgtggacccccgtccgggtccggaacgcgatctggtgctgggtcgccctggccgtcgtcgtcggcagtgtcgtggtctgcgacatcgcctggggtgacctggtcaccgtgtggggcaagatccccgccgtcgccgtgcagttctggccgccgtccttcggcagctacggtgccggcatcatgttcagcgcgatgttcgacaccgtcgcgatctccctcgccgcgaccgtgctcgcgctgctgctctcgctcgtcatcgggtccctggccgcccgcaacgtcgctcccgggcgcggggtccggggcggcgcacggctgctgctcgtcgtcgtccgcgggatcccggagacgatcctcgcggtcgtgctcatcgtcgtgacggggctcggtgcgcaggcgggcaccctcgccctggccttcggtggcatcgggctcctcggcaagctgatcgcggactcgctcgaagaggtcgctcccggaccggagcgcgccgtcacggccaccggcgcgggtcgcctgcagcgctacgcctcggcgacggtgccgcaggggttccgcgcgatgctcgggcacaccttctacctgctcgacacgaacatccgtgcggcgacgatcctcggcatcgtcggaggtggcggcatcggctactacctgctcaacgcggcgcagggttcgaactacgcgtccgtcaccgcgatcgtcctgctcatcctggtcacggtgctcgtcaccgagggcctggccatgtggctgcggcgggtgttccggtga

SEQ ID NO: 30 - DNA, 1725 bp, Curtobacterium sp.
  source 1..1725: mol_type = genomic DNA
atgacggccctcggggcaccgcgcgtcagccgggccggcgacgtcgacgcccgcgcgccccgccgaccggtcgcggtccagcggatcgtcgccggggtcgtggtgctcgccatggtcgccttcggggcgtggtcgctgtccgagctcgacttcggctggagcgccgtcccgacgagcatcgccaacgcagcgcgggtgttctcccagatggacccgatcagcttccccgcgccgggcgacctggtctacctcatcggcctcacgatcggcatcgtcatcctcggcaccgtcgtcgccgcgctgatctcggtgccggtcgcctggctcgccgcggccaacacgacgcccgtcggctggtcgcgctgggtcggacgggcgatcggtgcgctgacccgcgcggtgccggacgtcgtgatcgcgctggccttctcgctcaccttcgcgctgggcagcccgctgccgggcatcctggcgctcggcatccactcgatcgggatgatctccaagctcttcgccgacgccgtcgagcagatcgacgacgggccccggctcgcgatcagggccgccggtggctcccgcgcccaggagttctggtccggcgtcgtcccgcaggtgctccccgcgtggatcgcgatcgtcctgcaccggttcgacatcaacctccgcggctccgccatcctcgggtacgccggggtcggcgggctcgggtacgcgatgaagatcgcgttcgggcagttcccgaccgggtacggacgcggactcggcatcgcgatcgtcatcttcgcgctctgcgtcgtgctcgaggtcgtctcgtcgacgatccgccgcaccctgctcggcgtcgagccgtccggccgcggcctcggtgaccggatcgtccgccgcgcgaccaggaaccggccagctccgaccggacgcgtcgccggcggtgtcaccgtggactcgatgtcccgccgaccgtggacaccccgtcgggtgcggaacaccgtgtggtcgctcgcggccgtcgcgctcgtggtctcgagctggtggatggccggcgtcgacttcgacgacatcgcgtggaaccaggtggtgcccacgctgcagagcttctggccgccgaacctcggcagccacacgttcggcgagttcgcgcaggcgctgctcgtgacggtgcaggtcgccttcgcggccgcgctcctctcgctcgtcctgtcgctggtcatcgggtcgctcgcggcacgcaacgtcgcgccgaacggtgccgtccggagcgtgttccgggtcgtgctcgtcgtgttccgcggtgtccccgaactcgtgctggcgatcttcctgatcatggtgacggggctcggcaaccaggccggcgtcgtcgcgctcgcgttcggtgggatcgggctgctcggcaagctcgtcgccgactccttcgaggaggtgccgtccggaccggagcaggccctcacggccgccggggcgacgcgtgggcagcggtacctcgcggccacgctcccccaggggctgccgtcgttggtgggcaactcgctgtacctcgtcgacacgaacatccgcgcggcgacgctgctcgggatcgtcggcggcggcggggtcggcttctacctgacgaacgcgtccaccgtcctcaccctgcacggtcaggtgacgaccctcgtggccatggtgctcgtcgccgtgctcgcggtcgagggcctggcggcgtggatgcggcgggtgtaccggtga

SEQ ID NO: 31 - DNA, 1725 bp, Curtobacterium sp.; identical to SEQ ID NO: 30
  source 1..1725: mol_type = genomic DNA

SEQ ID NO: 32 - DNA, 1755 bp, Curtobacterium sp.
  source 1..1755: mol_type = genomic DNA
atgaccgccgtcgccgagcggcccgccgtcggctcccaccgttcgcccgagcaggccgtcgcggcggcaccacgccgacgcgtgtcgccggagcgcgtcgccgccgggctgaccctcgtcgccctggccgtgctggcgtgcgtcgccctggcccggatcgacatctcggtgccgaacatggtcgcgtcgtggtcgaacgcggagcacttcttccagcgggtcggcaccgtccggttccccgacgcggcgacgctcctgcagcagaccgcgctcaccgtcggtctcgtgctgctcggcaccctgctcgccgccgtgctgagcgtgcccgtcgcgtacttcgccgccggcaacaccagcccggggcgcgggtgggtcgctgccgcacggttcgtgagcgtcctgacccgcgcggtcccggacgtcgtgctcgcgatggtgttcgtgctgctgttcacgatcggaccgctgcccggcatcctcgccatcggcatccactcgatcgggatgatctcgaagctgttcgccgacgccatcgagcagatcgacgagggaccgcgcaccgcgatccgcgccaccgggggcacccggctgcagcagttcaccgccggtgtcctgccccaggtgctgccgtcgtgggtcgcgaccgtcctgcaccgcaacgacatcaacctgcgcgggtccgtcatcctgggatacgtcggcatcgtcgggctgggccgcgagatgtcgttcgcgttcaagtccctcgactactcgctcggcatcgggtacgcgatcgtcatcttcgcgctctgcgtgctcatggagatcgtgtcgagctcggtccgcgccgccatgctcggcatcgcgccgaccggccgggggatcggcgcgagcgcgatccgggcacgcatgcgccgccggcaggccggcaacgcaccgaccacgcggcacggtgccgccaccacaccgcgcttcgccaccccggaggacgccctccgccgcccgtggaccgccgcccgcgtccgcacgaccgtgtggtcctgggtcgcgatcctcgtcgtgatcggcagcgtcgtcgtgtgcgacatcgcccggggcgacctcggcaccgtgtgggcgaagatcccgccggtcgccgccgggttctggccgccgtcgttcggcagctacggtgccggcgtcatggtctcggcgatgttcgacgccgtcgccatcgcgctcgccgccaccctgctcgcgctcgtgttctcgatggtcatcggctccttcgcggcacgcaacgtcgcaccgaccggcggcgtccggaccggcgcacggttcctgctggtggtcgtgcgcggcatcccggagacgatcctggccgtggtgctcatcgtcatcaccggcctcggcgcccaggcgggcaccctcgcactggcgttcggcggcatcggcctgctcggcaagctcgtcgccgactcgctggaagaggtgcagcccggaccggagcgtgccctcaccgccacgggcgcgacgcgcttccagcgctacgccggggccacggtgccgcagggcatccgcgcgatgatcggtcacacgttctacctcgtcgacacgaacatccgcgccgcgacgatcctcggcatcgtcggtggtggtgggatcggctactacctgctcaacgccgcgcagggctcgaactacgcgtcggtcaccgggaccgtgctcatgatcctggccacggtgctcgtcgtcgagggcatcgccatgtggatgcggagggtcttccgatga

SEQ ID NO: 33 - DNA, 1725 bp, Curtobacterium sp.; identical to SEQ ID NO: 30
  source 1..1725: mol_type = genomic DNA

SEQ ID NO: 34 - DNA, 1048 bp, Curtobacterium sp.
  source 1..1048: mol_type = genomic DNA
gtgaacatcaagcgaatcggttcgatcgcagcaatcgcgatcgccggcgcagtcgtgctctcctcgtgcgcggcgaacgaggacgcgggcagcaccgagtcctcctccaccagcggcagcgactactcgaagctctccggcaccctgaccggctccggctcgtcggcacagcagaccgcgcaggccacctgggccgccggtttccagaacgaggcctcgggcgtcaccgtgaactacaccccggacggctccggcgcaggccgcgagaacttcatgtcgggtgccgcggacttcgccggctccgacgccgcgctgaaggacgaggagctctcgggcaagttcgagtcctgcaaggcggacaccaagggcatcgacatccccgtctacatctccccgatcgccatcgcgtacaaggtcgacggcgtcagcgacctgacgctcgacgcgaagaccatcgcgggcatcttctcgggcaagatcaccaagtgggacgactccgagatcgccgacctgaacaaggacgcgaagctgccggacgccgacatcacggtcgtccaccgctcggacgactcgggcaccacgcagaacttctcggagtacgtctccgcgaacgcttctgacgtctggaccgaagagcccagccagaccttcccgtactcggtcggtgacagcgcgaagggcacctccggtgtcgcctccgcgatgggcaacgccaccaacgccatcacctacatcgacgactccggtgcgggcgacctcgaccgcgcgaagctgatggtcggcgacaaggcgaccgagatctcggccgacggcgccgcgcaggtcgtcgccgactcgaagatcgcgaccggccgtgaggacaacgacctcgcgatcgacatcgaccgcaaggacaccgctgacggcgcctggccgctggtcctcgtctcctacgccatcgcgtgccaggagtacaaggacgccgacaaggccgagctcgtgaagggctacctcgactacgtcgtctcgagcgccgctcaggacgccgctgccaaggaggccaagtcgg

SEQ ID NO: 35 - DNA, 1110 bp, Curtobacterium sp.
  source 1..1110: mol_type = genomic DNA
gtgaacatcaagcgaatcggttcgatcgcagcaatcgcgatcgccggcgcagtcgtgctctcctcgtgcgcggcgaacgaaggcggcgcgggcagcaccacgtcggcctccgcctcgggtgtcgactactcgaagctctccggcacgctcaccgcgtcgggctcctccgcacagcagaccgccgagaccgtctggggcaccggcttccaggaccaggcctccggcgtcacggtcaactactcgccggacggctccggcgccggccgcaagaacttcatctccggcgcggcggacttcgccggctcggacgcagcgctctccgacgaggagctctccggcaccttcgcgaactgcgcggcggactccaaggccatcgacatcccggtctacatctccccgatcgccatcgccttcaaggtcgacggcgtcaaggacctgacgctcgacgccaagaccatcgcgggcatcttctccggcaagatcaccaagtgggacgactcccagatcgccgccctgaacaaggacgcgaagctgccgagcgcgaacatcaccgtcgtgcaccgttcggacgactcgggcaccacgcagaacttctccgagtacctgtcggcgaacgccggtgacgtctggaccgaagcgcccagccagaccttcccgtaccaggtcggcgacggcgctgcgaagggctccggcgtggcctcggccatgcagggcgcgaccaacgcgatcacctacatcgacgactccggtgcaggcgacctcgacaaggccaagctcatggtcggcgacaaggccaccgagctctccgccgacggcgccgccaaggtcgtcgcggactccgagacggtctccggccgcgagacgaacgacctcgcgatcgacatcaaccgcaaggacaccgcggacggcgcctggccgctcgtgctcgtctcctacgccatcgcgtgccaggagtacaaggacgccgacaaggccgacctggtcaagtcgtacctgacctacgtcgtgtccgacgacgcccagaaggccgccgcgacggaggccaagtcggccgccctctcctcggacctcggcaagaaggccgctgacgcggtcgcctcgatcaagtaa

SEQ ID NO: 36 - DNA, 1110 bp, Curtobacterium sp.; identical to SEQ ID NO: 35
  source 1..1110: mol_type = genomic DNA

SEQ ID NO: 37 - DNA, 1104 bp, Curtobacterium sp.
  source 1..1104: mol_type = genomic DNA
gtgaacatcaagcgactcggttcgatcgcagcgatcgcgatcgtcggcgccgtcgtgctctcctcctgcgcggcgaacgagggcggcagcggcgaggccgctcccacctcgagctccgggaccgactactcgaagctctcgggcacgctgaccggctccggctcgtccgcacagcagaccgccgaggccacctgggccgccggcttccagaacgtcgcctccggcgtgacggtcaactactcgccggacggctccggcgccgggcgcaagaacttcatctcgggtgccgcggacttcgccggctccgacgccgcgctcaaggacgaggagctctcgggctccttcggtctctgcgcggacaaggccatcgacatcccggtctacatctccccgatcgccatcgcctacaaggtggacggcgtctccgacctgacgctcgacgcgaagaccatcgcgggcatcttctcgggcaagatcaccaagtgggacgacccccagatcgccgcgctgaacgacggcgcgaagctgccgtcctcgaacatcaccgtcgtccaccgttcggacgactcgggcaccacgcagaacttctcggagtacgtctcggcgaacgcgtcggacgtctggaccgaggagccgagccagacgttcccgtaccaggtcggtgactcggcgaagggcacctcgggtgtcgcgtcggccatggccagcgcctcgaacgccatcacgtacatcgacgactccggcgccggtgacctcgacaaggccaagctcatggtcggcgacacggccaccgagatctcggccgagggcgctgccaaggtcgtcgcggactccgagacggtctccggccgcgagacgaacgacctcgcgatcgacatcaaccgcaaggacaccgccgagggtgcgtggccgctggtcctcgtctcctacgcgatcgcctgccaggagtacaaggactccgcgaagggtgagctcgtgaagggttacctcgactacgtcgtctcgaaggacgcacaggagtccgccgcgaaggaggccaagtcggccgccctctcctccgacctggcggagaaggctgcgaaggccgtcgcctccatcaagtaa

SEQ ID NO: 38 - DNA, 1113 bp, Curtobacterium sp.
  source 1..1113: mol_type = genomic DNA
gtgaacatcaagcgaatcggcacggtcgcggcaatcgcgatcgccggcgcagtcgtgctctcctcgtgcgcggcgaacgagggcgcgggcagcaccgcaccctcctcttcctccagctcgggcaccgactactcgaagctctccggcaccctcaccggttcgggctcgtccgctcagcagacggccgaggccacctgggccgccggcatccagaacgtcgcctccggcctcacggtcaactacacgcccgacggctccggcgccggccgcaagaacttcatgtcgggtgccgcggacttcgccggctccgacgccgccctgaaggacgaggagctctccggctcgttcgccctctgcgccgcggactcgaaggccatcgacatcccggtctacatctccccgatcgccatcgcctacaaggtcgacggcgtcaaggacctcacgctcgacgcgaagaccatcgccgggatcttctcgggcaagatcaccaagtggaacgactcgcagatcgcctccatcaacaaggacgcgaagctgccggacgccaacatcacggtcgtccaccgctccgacgactcgggcaccacgcagaacttctccgagtacgtctcggcgaacgccggcgacgtctggaccgaagagcccagccagacctacccgtaccaggtcggcgacagcgccaagggcacctcgggcgtggcctcggccatggccagcgcctcgaacgcgatcacctacatcgacgactccggtgccggtgacctcgacaaggccaagctcatggtcggcgacaaggccaccgagatctcggccgagggcgctgcgaaggtcgtctccgactccaaggtcgtcgaaggccgcgcggacaacgacctcgcgatcgacatcgaccgcaaggacactgccgacggcgcatggccgctcgtgctcgtctcctacgcgatcgcctgccaggagtacaaggacgccgacaagggcgagctcgtgaagggctacctcgactacgtcgtctcgaaggacgcgcaggaggcagccgcgaaggaggccaagtcggccgccctcacggacgacctctcggagaaggccgccaaggccgtcgcctccatcaagtaa

SEQ ID NO: 39 - DNA, 1104 bp, Curtobacterium sp.; identical to SEQ ID NO: 37
  source 1..1104: mol_type = genomic DNA

SEQ ID NO: 40 - DNA, 1110 bp, Curtobacterium sp.; identical to SEQ ID NO: 35
  source 1..1110: mol_type = genomic DNA

SEQ ID NO: 41 - DNA, 1086 bp, Curtobacterium sp.
  source 1..1086: mol_type = genomic DNA
atgtccctcgcgctccgtcagaccggcaccgccggcaacgtctacgccaacggcaagctgcacgcctcggtcccgtggctgctgctcgtcggcagctgggtcgccctcgtcctggtcttcgccctgctcaacgcgggcggcgccgtcaaggacttcaacgtcgtcgccgcgatcttcctcggcacggtcctgttcgacgtcctcatcgtcgtcatctcgcgcatcgtcgagggtggccgcaaggcggtcgaccgcctggtcacctcgctcgtcatcacggcgttcgtcatcgcggtcctgccgctggtgtcgctgctctggacggtcatcgccaagggcgtcgcccgcttcgacgcgaacttcttctcgtactcgatgcgcggggtcatctccgagggcggcggcgcggtccacgccctgatcggcacgctcgagatcacgctgttcgcggcgctcatctcggtcccgatcggcctgctcacctcgatctacctggtcgagtacggtcgcggcgcactggcgaagggcatcacgttcttcgtcgacgtcatgacgggcatcccgtcgatcgtcgccggtctgttcgcctacgcgctcttcgcgctgttcctcggcccgggtgcccgcttcggcctggtcggctccgtcgccctgtcggtgctgatgatcccgatcgtcgtgcgctccaccgaagaggtgctgaagatcgtcccgatggaactccgcgaggcctcgtacgccctcggggtgccgaagtacctcacgatcctcaaggtcgtgctccccaccagcctcgccggcatcaccaccggcgtgatgctctcgatcgcccgcgtcatcggtgagaccgccccgctgctcgtcacggccggcttcaccgcgagcatgaactacaacctgttccgtgacccgatgatgaccctgccggtgtacgcgtacacgcagtactcgcagcagggcgccaacccggtgccgttcgtcgaccgggcctggaccgcggctctggtgctcatcctgatcgtgatgctgctcaacctgctcgcccggttcatcacccgtctcttcgcccccaagctcagccgctga

SEQ ID NO: 42 - DNA, 1080 bp, Curtobacterium sp.
  source 1..1080: mol_type = genomic DNA
atgtccctcgcaatccgtccgaccggcggcaacgtcttcgccaacggcaagctgcacacctccgtgccgtggctgctcctcgccggcagctgggtcgccctggccctggtcttcgccctgctcaacgcaggcggcgccgtcaaggacttcaacgtcgtcgcggcggtcttcctcggcacggtcctgttcgacgtcctcatcgtggtcgtctcccggatcgtcgagggtgggcgcaaggcggtcgaccgcctcgtcacctcgctcgtcatcaccgcgttcgtcatcgcggtgctcccgctggtgtcgctgctctggacggtcctcgcggacggtctggcccggttcgacgtcgcgttcttctcgtactcgatgcgcggtgtcatctccgtcggtggtggcgccctgcacgccctgatcggcacgctcgagatcaccctgttcgcggccgtcatcagtgtcccgatcgggctgctcacgtcgatctacctggtcgagtacggccgcggcgccctcgcccgcggcatcacgttcttcgtcgacgtcatgacgggcatcccctcgatcgtggccggcctgttcgcgtactcgctcttcgcgctgttcctcggcccgggcgcccggttcggtctggtcggatcgatcgccctcgcggtcctgatgatcccgatcatcgtgcgcgccaccgaagaggtcctgaagatcgtgccgatggagctccgcgaggcctcctacgccctcggtgtcccgaagtggctgacgatcgtcaaggtcgtgctgcccacctcgctcgccggcatcacgacgggcgtcatgctcgcgatcgcccgcgtcatcggcgagaccgctcccctgctggtgaccgctggctacacgtcgagcatgaactacgacctgttcggcaacccgatgatgaccctgccggtgttcgcgtacacgcagtactcgcagcagggcgccaacccggtgccgttcgtcgaccgggcctggaccgcagcactcgtcctgatcctcatcgtgatgctgctgaacctgctcgcccggttcatcacgcgtctcttcgcccccaagctctcccgctag

SEQ ID NO: 43 - DNA, 1080 bp, Curtobacterium sp.; identical to SEQ ID NO: 42
  source 1..1080: mol_type = genomic DNA

SEQ ID NO: 44 - DNA, 1086 bp, Curtobacterium sp.
  source 1..1086: mol_type = genomic DNA
atgtccctcgctctccgtcagaccggtgtcgccggcaacgcgtacgccaacggcaagctgcacaagtccgtgccgtggctgctgctcatcggctcctgggtcgccctgatcgcggtcttcgccctgctcaacgccggcggcgccgtcaaggacttcaacgtcgtcgcggcgctgttcctcggcaccgtcctgttcgacgtcctcatcgtcgtcgtctcgcgcatcgtcgagggcggccgtcaggcggtcgaccggctgatcacctcgctggtcgtcaccgcgttcgtcatcgccgtcctgccgctcgtgtccctcctctggaccgtggtcgccgacggcgtcgcccgcttcgacgccacgtacttcagctcctcgatgcgcggtgtcatctccgagggcggcggcgccctgcacgccctgatcggcacgctcgagatcacgctcttcgcggcgctcatctcggtgccgatcggtctgctcacctcgatctacctcgtcgagtacggcaagggcgccctggcgaagggcatcacgttcttcgtcgacgtcatgacgggcatcccgtcgatcgtggccggtctgttcgcgtactcgctgttcgcgctcgtcctcggcccgggcgcccgcttcggcctggtcggctcgatcgcgctgagcgtgctcatgatcccggtggtcgtccgctccaccgaagaggtcctgaagatcgtgccgatggaactccgcgaggcctcctacgcactcggcgtgccgaagtacctgacgatcctcaaggtcgtcctgccgacgagcctcgcgggcatcacgacgggtgtcatgctctccatcgcccgtgtcatcggcgagaccgccccgctgctcgtgaccgccgggttcacggcgagcatgaactacgacctgttccgggacccgatgatgacgctgccggtgttcgcgtacacgcagtactcgcagcagggcgccaacccggtaccgttcgtcgaccgcgcctggacggcagcgctcgtcctcatcctcatcgtgatgctgctcaacctgctcgcccgcttcatcacccgcctcttcgcccccaagctcccgcgctga

SEQ ID NO: 45 - DNA, 1080 bp, Curtobacterium sp.
  source 1..1080: mol_type = genomic DNA
atgtccctcgccctccgtcccaccggcatgaccggcaacgcctacgccaacggcaagctgcacaagtcggtgccgtggctcctcctcgtcggctcctgggtcgccctggtcctcgtcttcgccctgctgaacgccggcggcgccgtgaaggacttcaacgtcgtcgcggccctgttcctcggcaccgtcctgttcgacgtcctcatcgtcgtgatctcgcgcatcgtcgagggcgggcgcaaggcggtcgaccgtctcatcacgtcgctggtcgtcaccgcgttcgtcatcgcggtgctgccgctggtgtcgctgctctggacggtcctcgccgacggcctcgcccgcttcgacgcgaacttcttcacgtactcgatgcgcggcgtcatctccgagggtggcggcgccgtgcacgccctcatcggcacgctcgagatcacgctgttcgcggcgctcatctcggtgccgatcggcctgctcacatcgatctacctcgtcgagtacggcaagggcgcgctcgcgaagggcatcacgttcttcgtggacgtcatgaccggcatcccctcgatcgtcgccggcctgttcgcctactcgctgttcgcgctgttcctcggccccggcgcccgcttcgggctggtcggctcggtcgcgctgagcgtgctgatgatcccgatcgtcgtgcgctccaccgaagaggtgctgaagatcgtgccgatggagctccgcgaggcctcgtacgccctcggcgtcccgaagtacctcaccatcctgaaggtcgtcctgcccaccgccctcgccggcatcaccaccggcgtcatgctctccatcgcccgtgtcatcggcgagaccgcaccgctgctggtcacggccgggttcaccgcgagcatgaactacgacctgttcaaggacccgatgatgacgctgccggtgttcgcgtacacgcagtactcgcagcagggcgccaacccggtgccgttcgtcgaccgggcctggaccgcggcgctgctgctcatcatcatcgtgatgctgctcaacctgctcgcccggttcatcacccgcctcttcgcccccaagctcagc

SEQ ID NO: 46 - DNA, 1086 bp, Curtobacterium sp.; identical to SEQ ID NO: 44
  source 1..1086: mol_type = genomic DNA

SEQ ID NO: 47 - DNA, 1080 bp, Curtobacterium sp.; identical to SEQ ID NO: 42
  source 1..1080: mol_type = genomic DNA

SEQ ID NO: 48 - DNA, 780 bp, Curtobacterium sp.
  source 1..780: mol_type = genomic DNA
gtgtccaagcgcatcgaggtcgacggcctcaacgtctactactcgaagttcaaggcggtcgagggtgtcgacatcaccatcgagccccgcaccgtcacggccttcatcggcccgtcgggctgcggcaagtccaccttcctccgcaccctgaaccgcatgcacgaggtcatccccggcgcatgggtcgagggctcggtcaagatcgacggcgacgacctgtacggcgccggcgtcgaccccgtgctcgtccgtcgtcaggtcggcatggtcttccagcgtccgaacccgttccccacgatgtcgatccgcgacaacgtgctggcgggcgtgaagctcaacaacaagcgtgtctcccgctccgaggccgacgacatcgtcgagcgctcgctgcagggtgcgaacctgtggaacgaggtcaaggaccgcctcgacaagcccggcatgggtctgtccggtgggcagcagcagcgtctctgcatcgcccgcgcgatcgcggtcgagccggacgtgctcctgatggacgagccctgctcggccctcgacccgatctcgaccctcgccatcgaggacctgatcgaggagctcaagaaggagttcacgattgtgatcgtgacccacaacatgcagcaggcgtcgcgcgtcagcgacaagacggcgttcttcaacatcgccggtaccggcgcccccggcaagctcatcgagttcgacgacaccgcgacgatgttctcgaacccgtccgtgcaggccaccgaggactacgtctcgggccgcttcggttga

SEQ ID NO: 49 - DNA, 780 bp, Curtobacterium sp.
  source 1..780: mol_type = genomic DNA
gtgtccaagcgcatcgaggtcgacggcctcaacgtctactactcgaagttcaaggcggtcgagggcgtcgacatgacgatcgagccccgcaccgtcaccgccttcatcggcccgtccggctgcggcaagtccacgttcctccgcacgctcaaccgcatgcacgaggtcatccccggcgcctacgtcgagggctcggtcaaggtggacggcgacgacctctacgcccccggcgtggacccggtcatcgtccggcgccaggtcggcatggtgttccagcgcccgaacccgttcccgacgatgtccatcaaggacaacgtgctcgccggcgtgaagctcaacaacaagcgcgtcagccgctccgaggcggacgacatcgtcgagcgctccctgcagggcgcgaacctgtggaacgaggtcaaggaccgcctcgacaagcccggcatgggactgtccggcggccagcagcagcgtctctgcatcgcgcgcgccatcgccgtgcagcccgacgtcctgctgatggacgagccgtgctccgcgctcgacccgatctcgaccctcgccatcgaggacctgatcggcgagctcaagaaggagttcacgatcgtgatcgtgacccacaacatgcagcaggcctcgcgcgtcagcgacaagaccgcgttcttcaacatcgcgggcaccggccagcccggtcacctcatcgagtacgacgacaccgcgacgatcttctcgaacccgtccgtcaagtcgaccgaggactacgtctccggccgcttcggttga

SEQ ID NO: 50 - DNA, 780 bp, Curtobacterium sp.; identical to SEQ ID NO: 49
  source 1..780: mol_type = genomic DNA

SEQ ID NO: 51 - DNA, 780 bp, Curtobacterium sp.
  source 1..780: mol_type = genomic DNA
gtgtccaagcgcatcgaggtcgacggcctcaacgtctactactcgaagttcaaggccgtcgagggcgtcgacatgacgatcgagccccgcaccgtcaccgccttcatcggcccgtcgggctgcggcaagtccaccttcctccgcacgctcaaccgcatgcacgaggtcatccccggcgcgtacgtcgagggctccgtcaagatcgacggcgacgacctgtacggccagggcgtcgacccggtgctcgtccggcgccaggtcggcatggtgttccagcgcccgaacccgttcccgacgatgtcgatccgcgacaacgtcctggcaggcgtcaagctcaacaacaagcgcgtctcgaagtccgaggccgacgacgtcgtcgagcgctccctccagggtgcgaacctgtggaacgaggtcaaggaccgcctcgagaagcccggcatgggcctgtccggtgggcagcagcagcgtctctgcatcgcccgcgcgatcgcggtgcagccggacgtcctgctcatggacgagccgtgctcggcgctcgacccgatctcgacactcgcgatcgaggacctgatcgaggagctcaagaaggagttcaccatcgtcatcgtcacgcacaacatgcagcaggcctcgcgcgtcagcgacaagaccgcgttcttcaacatcgcgggcaccggtgcgcccggcaagctcatcgagttcgacgacaccgccacgatgttctcgaacccgtcggtgcaggcgaccgaggactacgtctcgggtcgcttcgggtga

SEQ ID NO: 52 - DNA, 780 bp, Curtobacterium sp.
  source 1..780: mol_type = genomic DNA
gtgtccaagcgcatcgaggtcgacggcctcaacgtctactactcgaagttcaaggcggtcgagggcgtcgacatcacgatcgagccccgcaccgtgaccgcgttcatcggtccgtccggctgcggcaagtccacgttcctccgcacgctcaaccgcatgcacgaggtcatccccggcgcctacgtcgaggggtcggtcaaggtcgacggcgacgacctgtacggccccggggtcgacccggtgctcgtccgtcggcaggtcggcatggtcttccagcggccgaacccgttcccgacgatgtcgatcaaggacaacgtcctcgccggcgtgaagctcaacaacaagcgcatgtcccgctccgaggcggacgacgtggtcgagcggtccctgcagggcgcgaacctctggaacgaggtcaaggaccgcctcgacaagcccggcatgggcctgtccggtggtcagcagcagcgcctctgcatcgcgcgggcgatcgccgtgcagcccgacgtgctgctgatggacgagccgtgctccgcgctcgacccgatctcgaccctcgccatcgaggacctgatcgaggagctcaagaaggagttcacgatcgtgatcgtgacccacaacatgcagcaggcctcgcgcgtcagcgacaagacggcgttcttcaacatcgccggcaccggacacccgggcaagctcatcgagtacgacgacaccgcgacgatcttctcgaacccgtccgtgcaggccaccgaggactacgtctccggccgcttcggttga

SEQ ID NO: 53 - DNA, 780 bp, Curtobacterium sp.; identical to SEQ ID NO: 51
  source 1..780: mol_type = genomic DNA

SEQ ID NO: 54 - DNA, 780 bp, Curtobacterium sp.; identical to SEQ ID NO: 49
  source 1..780: mol_type = genomic DNA

SEQ ID NO: 55 - DNA, 951 bp, Curtobacterium sp.
  source 1..951: mol_type = genomic DNA
atgacgaccgcaccggcccagccaggggccaccgtcaccccgaccaagccgaagcccgtcgtccgtgtcggcgaccgcgtcttctccgccgcctcggtcatcgccggcggcctcatcctcttcgtgctcgtgctcgtcgccgcgttcctcgtctggcagagcatcccggccttctccgccaaggtcggcgagctgccgggcaaggccacgaacttctgggacttcgtcggccccctggtcttcggcaccgtctggtccgcgctcatcgcgctcgtgatcgccgtgccgctgtccctcgggatcgccctcttcatctcgcacttcgcgccgcgtcgactcgcgccggtcctcggctacgtgatcgacctgctggccgcggtgccgtcggtcgtctacggcctctggggcatcgccgtgctggcgcccctggtgaagccgttctacgtcttcctgaacgagtacatcggctggttcccgctgttctccggccaggtgtccggcaccggtcgcacgatcctgaccgcgtcgatcgtcctcgccgtcatggcgatcccgatcatgaccgcggtcatgcgtgagatcttcctgcaggcgccgaccctcaacgaggaagcggcactggccctcggcgcgacccgctgggagatgatccgcctgtcggtcctgccgttcgcgaagtccggcatcgtctccgcgatcatgctcggcctcggtcgtgcactcggcgagacgatggcgatcgcgctggtgctgtcggtctcgacgaacgtcacgttccagatgctgacgtcgcagaacccctcgacgatcgccgcgaacatcgccctgcagttcgccgaggcatccggtacggccctgaacgcgctcatcgcctcgggtctgatcctcttcgtcatcaccctggtcatcaacatgctcgcgcggtacatcgtccgcagccgagtcagctga

SEQ ID NO: 56 - DNA, 951 bp, Curtobacterium sp.
  source 1..951: mol_type = genomic DNA
atgacctccgcacccgtccagcaaggcgcttccctcgccccggcgaagcccaaggccgtcgtccgcgtcggcgaccgtgtcttctccgccgcctccgtcatctccggcggcctcatcctggtcgtgctcgcgctggtcgccgccttcctcgtctggcagagcatccccgcgttctccgccaaggtcggcgagctgcccaaccaggccgcgaacttctgggactacgtcgggccgctcgtcttcggcaccatctggtcggcgctcctcgcgctcgtcatggccgtcccgctcgcgctcgggatcgcgctcttcatctcgcacttcgcgccgcggcgcatcgccccgctgctcggctacgtcatcgacctgctcgccgcggtcccgtcggtcgtctacggcctgtggggcatcgtggtgctgtcgcagttcgtcaagccgttctacgcgttcctcaacgagtacttcgcgtggatcccgctcttctccgggcaggtgtccggcaccggtcggacgatcctcacggcgtcgatcgtcctcgccgtcatgacgatcccgatcatgacggcgatcatgcgcgagatcttcctgcaggcgccgaccctcaacgaagaggccgcactcgccctcggcgccacgcgctgggagatgatccgcctgtcggtcctgccgttcgccaagtccggcatcgtgtccgcgatcatgctcggcctcggacgagcgctcggcgagacgatggcgatcgccctcgtgctgtcggtgtcgacgaacatctcgttcaagctgctcacgtccctcaacccgtcgaccatcgcggcgaacatcgccctgcagttcgccgaggcgtccggaacggccctgaacgcactcatcgcgtccggtctgatcctcttcgtcatcaccctggtcatcaacatgctcgcgcgctacatcgtgcgccgacgcgtcagctga

SEQ ID NO: 57 - DNA, 951 bp, Curtobacterium sp.; identical to SEQ ID NO: 56
  source 1..951: mol_type = genomic DNA

SEQ ID NO: 58 - DNA, 951 bp, Curtobacterium sp.
  source 1..951: mol_type = genomic DNA
atgacgaccgcaccggcccagccaggggccaccgtctcccccaccaagccgaaggccgtcgtccgcatcggcgaccgggtgttctcgaccgcctccgtggtcgcgggcagcctcatcctcttcgtgctcgtgctcgtggccgccttcctggtgtggcagagcatccccgccttcacggcgaaggtcggcgaactgccgaacaacgcgacgagcttctgggactacgtcggtccgctcgtcttcggcaccgtctggtccgcgctcatcgcgctcgtcatggccgtgccgctctcgctcggcatcgcactcttcatctcgcactacgcaccccgccgcatcgccccgctgctcggctacgtcatcgacctgctggccgcggtcccgtcggtcgtctacggcctctggggcatcgtcgtgctcgcgccgctcctcaagccgctgtacggcttcctcaacgagcacctcggctggatcccgctgttctccgggcaggtctccggcaccggccgcaccatcctgaccgcctcgatcgtcctcgcggtgatggcgatcccgatcatgaccgcggtcatgcgcgagatcttcctgcaggcgccgaccctcaacgaggaggcggcgctcgccctcggtgcgacgcgctgggagatgatccgcctgtcggtcctgccgttcgcgaagtccggcatcgtgtccgcgatcatgctcggtctcggccgcgccctcggggagacgatggcgatcgccctggtgctgtcggtctcgacgaacgtcaccttccagatgctgacgtcgctcaacccctcgacgatcgcggcgaacatcgccctgcagttcgccgaggcgtccgggacagcgctcaacgccctcgtcgcctcgggcctgatcctcttcgtcatcaccctggtcatcaacatgctggcgcgctacgtcgtgcgcacgcgggtctcctga

SEQ ID NO: 59 - DNA, 948 bp, Curtobacterium sp.
  source 1..948: mol_type = genomic DNA
atgacgaccgcaccggcccagccagtggcccccgtcaccccgaagccgaaggccgtcgtccgtgtcggcgaccgggtcttctccgccgcctcggtcatcgccggcgggctgatcctcttcgtcctcgtgctcgtcgccgccttcctggtctggcagagcatcccggcgttcaccgccaaggtcggcgagctgccgaaccaggccaccaacttctgggactacgtcggccccctcgtcttcggcaccgtctggtccgcgctcatcgcgctcgtgatcgccgtgccgctgtcgctcgggatcgccctcttcatctcgcactacgcaccccgccgcatcgccccggtgctcggctacgtgatcgacctgctggccgcggtgccgtcggtcgtctacggcctctggggcatcgtggtgctggcgccgttcgtgaagcccttctacgggttcctcaacgagtacctcggctggatcccgttcttctccgggcaggtgtccggcaccggccgcacgatcctcaccgcgtcgatcgtcctcgccgtcatggccatcccgatcatgaccgcggtcatgcgtgagatcttcctgcaggcaccgcgcctcaacgaggaagccgccctggccctcggcgcgacccggtgggagatgatccggatgtcggtcctgccgttcgcgaagtccggcatcgtgtccgcgatcatgctcggcctcggccgcgcgctcggcgagacgatggcgatcgcgctggtgctgtcggtgtccacgaacgtcaccttccagatgctcacctcgctgaacccgtcgacgatcgcggcgaacatcgccctgcagttcgccgaggcgtccggcacggccctcaacgcactcatcgcctcgggtctgatcctcttcgtcatcaccctggtcatcaacatgctggcgcgctacatcgtgcgtcagcgggtctcctga

SEQ ID NO: 60 - DNA, 951 bp, Curtobacterium sp.; identical to SEQ ID NO: 58
  source 1..951: mol_type = genomic DNA

SEQ ID NO: 61 - DNA, 951 bp, Curtobacterium sp.; identical to SEQ ID NO: 56
  source 1..951: mol_type = genomic DNA

SEQ ID NO: 62 - DNA, 1695 bp, Curtobacterium sp.
  source 1..1695: mol_type = genomic DNA
atgcacacgcgaatgaaccactggtgggacgggatctccctgcgcaccaagatcaccgggatcacggtgctgctcgtcgcgctcggattgctcgtcgccggactcggcacgatgaccgtgctgtccacctacctcatgcagcagctcgacaacacggtcaagcagacgaccgagcagctcgagggccagaacatcagcgacggcgagcagtactgcaagctgtccgtcgtcgtctcgcagagcgcctacgtcgccgcgtacgacgccgacggtgaccggatctgccagacgaaggcgtcgagccggcccgacgtccgcgaagccaccctggtcgaggcctcgcaagcgaaccagcgcatctcgctctacgacagccagcacaaccacgaatggcgtgcgcagatcatcccggcgtcgctgcagaaccagtccgacggcaccaccgagaccggctacgtgctcatcgcggtctccagcgccggcaccgacgagaccatcggcaagttcaccgcgatcttcctcgggttcggtgcctcggtcatcctgctcggcgccatgctcacccggctgctcgtcaccgcgaccttcgacccgctgcgcgacgtcgaggacaccgccgcacggttcgccgcgggcgacttcaaccagcgcctcgaggccgacaccccgaacaccgaggtcggccgcctcaaccgctccctcaacgtcatgctcgagcggatcgactccgcgttcgaggaccgcgagcgcaccatcgcccagatgcgccggttcgtcggggacgcctcgcacgagctccgcacgccgctcgtgtccctgcgcgggtacgccgagctgtaccggatgggtgccctgcgcaaggaagaggacgtcgcccaggcgatggagcgcatcgagaaggaagcgcagcggatgggcctgctcgtgcaggacctgctgcagctcgcccgcatcgacgagtcgaagccgctcgagctcggcccggtcgacctcgtcgccatcgcccgtgactcggcgctcgacacgatggcctccaaccccgaccgcgagatccaggtcctcgtcgaggactccctgaccggtgagagcgtgccccaggccgtgcgcccatcggcgtcctcgtccatcggctctccggactccggcgaagcaccgcccgtgtcgcccacctcggcgaacaccaccgggccgatcgcgttctcgcgccagaccatcgcgcgcctgcgggcccgtcgcacccgcgccatgaacatcgacgccggtgccgcaccgaccgacgagaccacaccgctgcccacggtcgtgctgccgaagcgcccgccgatcgtcctggcggaagagaacaagatccgccagatcgtcacgaacctgatgggcaacgccatgcggttcacggcgaacgacgacccgatcgagatcggcatcggcgtggacgacgagcgcggcatggcacacctcgacgtgatcgaccacggtgagggcatcccgccgcagctgcgcgagaagatcttccagcgcttctggcgtgcggacacctcgcgcgcccgggacaccggcggctcgggcctcgggctcgccatcgtgtccggcatcgtcgcggcgcaccacggctccgtcgaggtcttcgacaccgagggcggcggcgccaccttccgcgtctggctccccctgctcccccgcgacttcgccgcctga

SEQ ID NO: 63 - DNA, 1701 bp, Curtobacterium sp.
  source 1..1701: mol_type = genomic DNA
atgcacacgcgaatgagccgctggtgggacgggatctccctgcgcaccaagatcaccgggatcacggtgctcctcgtcgcgctcgggctgctcgtcgcggggctcggcaccatgacggtgctgtccacctacctcatgcagcagctcgacaacacggtcaagcagacgaccgagcagctcgagggccagaacatcagcgacggacagcagtactgcacgctctccatcgtgctgtcgcagagcgcctacgtcgccgcctacgacgcccgcggcgaccagatctgccagacccgcgcctccagtcggcccgacatccgcagcctcgacttcaccgcggcgtcgcggacctcgcagcggttctcgctctacgacagccagcacaaccacgagtggcgcgcgcagatcatccccgcctcgctccagaaccagtcgagcggcagcaacgagaccggcttcgtgctcgtcgccgtgtcgagcgcggacaccgaccagaccatcgcgcgcttcacggtgatcttcctgggcttcggcatctcggtcatcctcctcggcgcgatgctgacccgactcctggtgaccgcgaccttcgaccccctgcgcgacgtcgaggacacggctgcacgcttcgctgcgggcgacttcaaccagcgactcgaggccgacaccccgaacaccgaggtcggccgcctcaaccggtcgctgaacgccatgctcgagcgcatcgactcggcgttcgaggaccgcgagcgcaccatcgcgcagatgcgtcgcttcgtcggtgacgcctcgcacgagctccgcacgccgctagtgtcgctgcgcgggtacgccgagctctaccggatgggcgcactgcgcaaggaggaggacgtcgcccaggcgatggaccgcatcgagaaggaggcccagcgcatgggcctgctcgtgcaggacctcctccagctcgcacgcatcgacgagtcgaagccgctggagctcgggccggtcgacctcgtggccatcgcccgcgactcggcgctcgacaccatggcgtcgaacccggaccgcgagatccaggtgctcgtcgaggacgcggtgacgggcatgagcgtgccgcaggccgtgcgcccgctgcccgccggcacctcggacacgggcgaggccccggtgtccccgcagtcggccaacacgaccggtccgatcgcgttctcgcggcagaccatcgcgcgtctgcgtgcacgtcgtggccgcgcgatgggcgtcgacgacgagacgccgaccgacgacacgacaccgctgcccaccgtcgtgctgccggcccgtccgccgatcgtgctggccgaggagaacaagatccgccaggtcgtgacgaacctgatgggcaacgccatgcggttcacgacgtacgacgacccgatcgagatcggcatcggcgtggacgacgaccgcggcatggcgcacatcgacgtcatcgaccacggcgagggcatcccgccgcagctgcgcgagaagatcttccagcgcttctggcgggccgacacctcgcgcgcacgggacaccggcggctcgggactcggcctggcgatcgtctccggcatcgtgcacgcacaccgcggctccgtcgaggtgttcgacaccgagggcggcggcgcgacgttccgcgtctggctccccctgctcccccgcgactacaccccggcgccgaagtcccccgcggcctga

SEQ ID NO: 64 - DNA, 1701 bp, Curtobacterium sp.; identical to SEQ ID NO: 63
  source 1..1701: mol_type = genomic DNA

SEQ ID NO: 65 - DNA, 1704 bp, Curtobacterium sp.
  source 1..1704: mol_type = genomic DNA
atgcactccagaatgaaccgctggtgggacggcatctccctgcgaaccaagatcaccggcatcacggtgctcctggtggcgctcgggctgctcgtcgccggcctcggcacgatgaccgtgctgtcgaactacctggtctcccgcctggacacccaggtcagcacgacgagctcgcagctcgagggccagaacgtcagcgacggggagcagtactgcaagctgtcggtcgtgctcgagaagagctcgtacgtcgccgcctacgacagcgacggcaagcagatctgcgacacgcagtccacctcgcagccggacatcgccaccgtcaagttctcgggtgccgcagcgtcggggaagcccttcagcctctacgacaagcagcacaaccacgagtggcgtgcccaggtcatcccggcgaacctgcagaacctgtcgaccggcgccaccgagaccggctacgtgctcgtcgccgtgtcgagcgccgacaccgaccagaccatcgcccgcttcacgttcatcttcctggggttcggcgcctcggtcatcctgctcggggccatgctcacccggctgctcgtcaccgcgaccttcgacccgctccgcgacgtcgaggacaccgccgcccgcttcgccgccggtgacttcaaccagcgcctcgacgccgacaccccgaacaccgaggtcggtcggctgaaccgatccctcaacgccatgctcgagcggatcgacagcgcgttcgaggaccgcgaacgcaccatcgcgcagatgcgccgcttcgtcggtgacgcctcccacgagctccgcacgccgctggtgtccctccgcgggtacgccgagctgtaccgcatgggggccctccgcaaggaggaggacgtcgcccaggcgatggagcgcatcgagaaggaggccaagcgcatggggctcctcgtgcaggacctcctccagctcgcgcgcatcgacgagtccaagccgctcgaactcgggccggtcgacctcgtcgccatcgcccgggactcggcgctcgacaccatggcgtccaaccccgaccgcgagatccaggtgctcgtcgaggacgtgccccaggccgtccgtgctccgtcggccttcggtcccgctgccacgcctgcccccgatgccgtcccgacctcgccgtccgcggggaccaacaccaccggcccgatcgcgttctcccgccagacaatcgcccgcctgcgtgctcgtcgtggccgcgcgatggacgtcgagtcggagtcgcagcccggtgccgagaccaccccgatgcccaccgtggtgctgccgaagcgcccgccgatcgtcctggccgaggagaacaagatccgccaggtcgtgacgaacctcatgggcaacgcgatgcgcttcaccgcgcacgacgaccccatcgagatcggcatcggcgtggacgacgaccggggcatggcgcacatcgacgtcatcgaccacggcgagggcatcccgccgcagctccgcgacaagatcttccagcgcttctggcgtgccgacacctcccgcgcgcgggacaccggcggctcgggcctcgggctcgccatcgtgtcgggcatcgtcgccgcacaccagggttcggtcgaggtcatcgacaccgtgggcggtggcgccaccttccgcatctggctccccctgctcccccgcgactacacccccaccccgctccccacccccgagccctga

SEQ ID NO: 66 - DNA, 696 bp, Curtobacterium sp.
  source 1..696: mol_type = genomic DNA
atgaccgatggccccaagatcctgatcgtcgacgacgaaccgaacatccgcgacctcctcacgacctcgttgcgcttcgccgggttcgccgtccgcgcggtcggcaacggtgcccaggcgatctccgccgtgctcgaggaggagcccgacctgatcatcctcgacgtcatgctccccgacatgaacggcttcggcgtcaccaagcgcctgcggtcgtcggggtacacctcgccgatcctgttcctcaccgcgaaggacgacaccgaggacaagatcacggggctcaccgtcggcggcgacgactacgtcaccaagccgttctcgctcgacgagatcgtcgcgcgcatcaaggccatcctccgtcgcacgatgaacgacgaagaggacgcgatcatccgtgccggcgagctcacgatggaccaggacacgcacgaggtcacgatcggtgacgcgcagatcgagctgtccccgacggagttcaagctgctccgctacctcatgctcaaccccaaccgcgtgctctcgaaggcgcagatcctcgaccacgtgtgggagtacgacttcaacggcgacgcgggcatcgtcgagtcgtacatctcgtacctccgccgcaagctcgaccagtactcgacggagccgatcatccagacgaagcgcggtttcggctacatgctgaaggcgaccaaggcgtcctga

SEQ ID NO: 67 - DNA, 696 bp, Curtobacterium sp.
  source 1..696: mol_type = genomic DNA
atgagcgatggccccaagatcctgatcgtcgacgacgagccgaacatccgcgacctcctgacgacctcgttgcgcttcgccggcttcgccgtgcgtgcggtgggcaacggtgcacaggcgatctccgccgtgctggaagaagagccggacctcatcatcctcgacgtgatgctccccgacatgaacggcttcggcgtcaccaagcgcctccgctcctcgggctacacctcgccgatcctgttcctcaccgcgaaggacgacaccgaggacaagatcaccggcctcaccgtcggcggcgacgactacgtcacgaagccgttctcgctcgacgagatcgtcgcccgcatcaaggccatcctccgtcgcaccatgaacgacgaagaggacgcgatcatccgcgccggcgaactgacgatggaccaggacacgcacgaggtcaccatcggcgacgcgcagatcgagctctccccgaccgagttcaagctgctccgctacctcatgctcaacccgaaccgcgtgctgtcgaaggcgcagatcctcgaccacgtgtgggagtacgacttcaacggcgacgccggcatcgtcgagtcctacatctcgtacctgcggcgcaagctcgaccagtactcggccgagccgatcatccagaccaagcgcggcttcggctacatgctcaaggcgtcgaaggcctcctag

SEQ ID NO: 68 - DNA, 705 bp, Curtobacterium sp.
  source 1..705: mol_type = genomic DNA
gtgaaccggaccgagcacgtccacaccctcggcaccggcggacgcacgccgctcgtcctgctggccgaggacgaccccgacgtccgcgagaccacgcagctcctgctgctgcggcgcgggtggcgggtgaccgcgacggccgacgggatcgaggcgctcgccgcactcgacgccgaggcccccgacatcgcggtcgtcgacgtggcgatgccgcggctgaacgggctcgacctgacgccgcgcctcgtcgaggtcggggtgccggtggtcctgctcaccgcgcgcagcctgcccctggacgaggtcaccggcctcggggcgggggcggacgactacgtcacgaagccgttcgacgcggacgtgctgtccgcccggctgcacgccgtgctgcgccgtcgacgggggaggaccgcggaggtcaccacggtcggggacgtccgcatcgacgtggtcggccggcgggtggagcgcgccggtgccgtcgtgccgctcagcgccgtggagttccgcgtgctcgaggcgctcctccggaaccgcggcgccgtcctcagccgtgagcaggtcgtcgcgatggcgtgggagagcacgtggcaggacctgcggatcgtcgacacgaacgtgcagcgcctccgtcggaagctcggtccgggtgtgatcgacaccgtccgcgggttcggctaccgcatcgaggtcgagccctcgtga